(19) United States
(12) Patent Application Publication
(10) Pub. No.: US 2026/0226475 A1
(43) Pub. Date: Aug. 6, 2026

(54) SMALL INTERFERING RNA TARGETING ACVR1C AND USES THEREOF

(71) Applicant: Sanegene Bio USA Inc., Woburn, MA (US)

(72) Inventors: Peng Zhang, Woburn, MA (US); Chunxue Zhou, Suzhou, Jiangsu (CN); Xiaowei Sun, Suzhou, Jiangsu (CN); Xiaochen Xu, Shanghai, Pudong New Area (CN); Shiyu Wang, Woburn, MA (US); Marc Abrams, Woburn, MA (US)

(21) Appl. No.: 19/460,924

(22) Filed: Jan. 27, 2026

(30) Foreign Application Priority Data
Jan. 27, 2025 (WO) PCT/CN2025/075542

(51) Int. Cl. C12N 15/113

(52) U.S. Cl. CPC C12N 15/1138; C12N 2310/14; C12N 2310/315; C12N 2310/321; C12N 2310/322; C12N 2310/351

(57) ABSTRACT

This disclosure relates to isolated oligonucleotides comprising duplex regions targeting human ACVR1C mRNA, and delivery systems, kits and compositions comprising the same, and methods of using the same for inhibiting or downregulating ACVR1C gene expression and treating or preventing a disease or disorder associated with aberrant or increased expression or activity of ACVR1C, or a disease or disorder where ACVR1C plays a role.

### RELATED APPLICATIONS

This application claims priority to, and the benefit of, International Application No. PCT/CN2025/075542, filed Jan. 27, 2025, the contents of which are incorporated herein by reference in their entirety.

### REFERENCE TO AN ELECTRONIC SEQUENCE LISTING

The contents of the electronic sequence listing (02203WO_CRF_sequencelisting.xml; Size: 7,425,476 bytes; and Date of Creation: Jan. 23, 2026) are herein incorporated by reference in its entirety.

### BACKGROUND

Activin A receptor (ACVR1C or ALK-7) is a type I receptor for the TGFB family of signaling molecules. Upon ligand binding, type I receptors phosphorylate cytoplasmic SMAD transcription factors, which then translocate to the nucleus and interact directly with DNA or in complex with other transcription factors.

ACVR1C plays a critical role in the pathogenesis of several diseases. ACVR1C has been reported to inhibit cell proliferation in several cancer types, including breast cancer, endometrial cancer, and ovarian cancer. In addition, ACVR1C plays a critical role in obesity and diabetes. ACVR1C loss has been associated with an increase in fat utilization, lowering of adiposity, and driving PPARG-regulated gene signatures, which are indicative of healthy adipose function.

Accordingly, there is a need for therapies for subjects having diseases, disorders, and symptoms associated with abnormal, or even baseline, ACVR1C expression levels.

### SUMMARY

The present disclosure provides compositions targeting ACVR1C and methods of reducing ACVR1C expression for treatment of subjects having a disease, disorder, or symptom associated with ACVR1C expression level. In some embodiments, the expression level is elevated. In some embodiments, the expression level of ACVR1C may not be elevated but suppression of its expression is of benefit.

The present disclosure provides an isolated double stranded oligonucleotide comprising an antisense strand comprising a sequence selected from SEQ ID NOs: 2-269 and its corresponding sense strand comprising a sequence selected from SEQ ID NOs: 538-805, as set forth in Table 5.

In some embodiments, the antisense strand comprises SEQ ID NO: 58 and its corresponding sense strand comprises SEQ ID NO: 594.

In some embodiments, the antisense strand comprises SEQ ID NO: 95 and its corresponding sense strand comprises SEQ ID NO: 631.

In some embodiments, the antisense strand comprises SEQ ID NO: 36 and its corresponding sense strand comprises SEQ ID NO: 572.

In some embodiments, the antisense strand comprises SEQ ID NO: 21 and its corresponding sense strand comprises SEQ ID NO: 557.

In some embodiments, the antisense strand comprises SEQ ID NO: 61 and its corresponding sense strand comprises SEQ ID NO: 597.

In some embodiments, the sense strand or the antisense strand or both comprise one or more modified nucleotide(s).

In some embodiments, the antisense strand comprises a modified sequence selected from SEQ ID NOs: 270-537 and SEQ ID NOs: 1074-1078, and its corresponding sense strand comprises a modified sequence selected from SEQ ID NOs: 806-1073 and SEQ ID NOs: 1079-1084, as set forth in Table 6.

In some embodiments, the antisense strand comprises SEQ ID NO: 1074 and its corresponding sense strand comprises SEQ ID NO: 1079.

In some embodiments, the antisense strand comprises SEQ ID NO: 1075 and its corresponding sense strand comprises SEQ ID NO: 1080.

In some embodiments, the antisense strand comprises SEQ ID NO: 1076 and its corresponding sense strand comprises SEQ ID NO: 1081.

In some embodiments, the antisense strand comprises SEQ ID NO: 1077 and its corresponding sense strand comprises SEQ ID NO: 1082.

In some embodiments, the antisense strand comprises SEQ ID NO: 1078 and its corresponding sense strand comprises SEQ ID NO: 1083.

In some embodiments, the antisense strand comprises SEQ ID NO: 1077 and its corresponding sense strand comprises SEQ ID NO: 1084.

In some embodiments, the antisense strand and its corresponding sense strand are selected from Table 7 or Table 8.

In some embodiments, the antisense strand comprises a phosphate mimic.

In some embodiments, the phosphate mimic is 5′-C-EP.

In some embodiments, the phosphate mimic is 5′-C-EPmUs.

In some embodiments, the sense strand comprises nucleotides modified with 2′-F modification, and nucleotides modified with 2′-O-methyl modification, according to the formula: 5′(M)₀(F)₀(M)₅(F)₁(M)₁(F)₄(M)₉ 3′ wherein “M” is a 2′-O-methyl modified nucleotide, and “F” is a 2′-F modified nucleotide.

In some embodiments, the antisense strand comprises nucleotides modified with 2′-F modification, and nucleotides modified with 2′-O-methyl modification, according to the formula: 3′(M)₀(F)₀(M)₆(F)₁(M)₁(F)₁(M)₃(F)₁(M)₂(F)₁(M)₁(F)₁(M)₁(F)₂(M)₁ 5′ wherein “M” is a 2′-O- methyl modified nucleotide, and “F” is a 2′-F modified nucleotide.

In some embodiments, the sense strand comprises phosphorothioate internucleotide linkages located between nucleotides at position 1 to 3 and position 19 to 20 from the first nucleotide at the 5′ terminus of the sense strand.

In some embodiments, the sense strand further comprises phosphorothioate internucleotide linkages located between nucleotides at position 18 to 19 and position 20 to a targeting ligand from the first nucleotide at the 5′ terminus of the sense strand.

In some embodiments, the antisense strand comprises phosphorothioate internucleotide linkages located between nucleotides at position 1 to 3 and position 20 to 22 from the first nucleotide at the 5′ terminus of the antisense strand.

In some embodiments, the sense strand or the antisense strand or both comprise a terminal or internal nucleotide linked to a targeting ligand.

In some embodiments, the targeting ligand is an adipose-targeting ligand.

In some embodiments, the adipose-targeting ligand is selected from a small molecule, a peptide, an antibody, or a carbohydrate.

In some embodiments, the targeting ligand is linked to a terminal nucleotide on the sense strand via a linker.

In some embodiments, the targeting ligand comprises at least one GalNAc G1b moiety, optionally three G1b moieties.

In some embodiments, the targeting ligand comprises G1b-BCN-C22PC2.

In some embodiments, the adipose-targeting ligand comprises a structure of

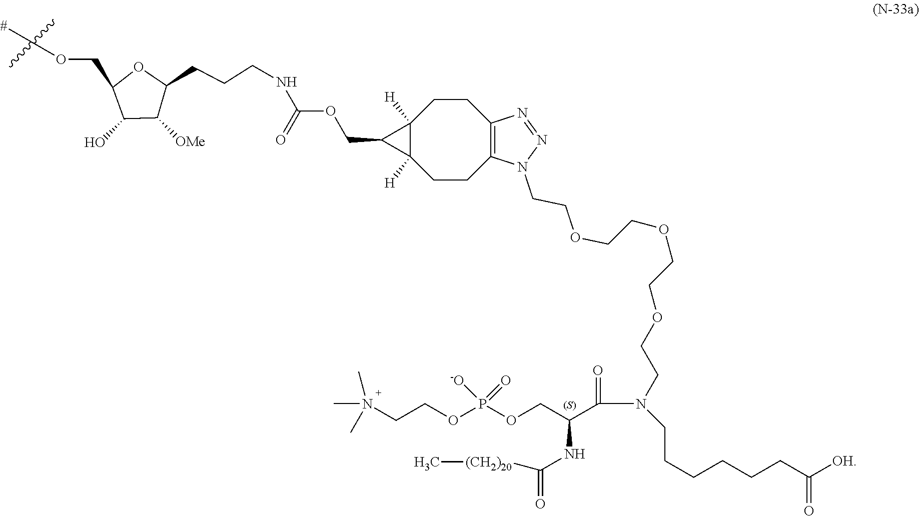

The present disclosure also provides a delivery system comprising the isolated double stranded oligonucleotide disclosed herein.

The present disclosure also provides a pharmaceutical composition comprising at least one isolated double stranded oligonucleotide disclosed herein or a delivery system disclosed herein, and a pharmaceutically acceptable carrier, diluent or excipient.

The present disclosure also provides a method of inhibiting or downregulating the expression or level of ACVR1C in a subject, wherein the method comprises administering to the subject in need thereof, an effective amount of at least one isolated double stranded oligonucleotide disclosed herein, a delivery system disclosed herein, or a pharmaceutical composition disclosed herein.

The present disclosure also provides a method of treating or preventing a disease or disorder associated with aberrant or increased expression or activity of ACVR1C or a disease or disorder where ACVR1C plays a role in the disease or disorder, wherein the method comprises administering to a subject in need thereof, an effective amount of at least one isolated double stranded oligonucleotide disclosed herein, a delivery system disclosed herein, or the pharmaceutical composition disclosed herein.

In some embodiments, the double stranded oligonucleotide is administered to the subject via an intravenous, a subcutaneous, an intraperitoneal, or an intramuscular route of administration.

The present disclosure also provides an isolated double stranded oligonucleotide disclosed herein, a delivery system disclosed herein, or a pharmaceutical composition disclosed herein for the treatment or prevention of a disease or disorder associated with aberrant or increased expression or activity of ACVR1C or a disease or disorder where ACVR1C plays a role in the disease or disorder.

The present disclosure also provides use of a isolated double stranded oligonucleotide disclosed herein, a delivery system disclosed herein, or a pharmaceutical composition disclosed herein in the manufacture of a medicament for the treatment or prevention of a disease or disorder associated with aberrant or increased expression or activity of ACVR1C or a disease or disorder where ACVR1C plays a role in the disease or disorder.

The present disclosure provides an isolated oligonucleotide comprising a sense strand and an antisense strand, wherein the antisense strand is substantially complementary to its corresponding sense strand such that the sense strand and the antisense strand form a double stranded region. In some embodiments, the double stranded region comprises an antisense strand selected from SEQ ID NOs: 2-269 and its corresponding sense strand selected from SEQ ID NOs: 538-805, as set forth in Table 5. As an example, the double stranded region comprises an antisense strand of nucleic acid sequence according to SEQ ID NO: 2 (5′ UUUUUCACAGCCACAUCUUCCC 3′), and a corresponding sense strand of nucleic acid sequence according to SEQ ID NO: 538 (5′ GAAGAUGUGGCUGUGAAAAA 3′), as set forth in Table 5. Moreover, SEQ ID NO: 2 and corresponding SEQ ID NO: 538 are identified as duplex number 1 (“Duplex No. 1”), as set forth in Table 5.

In some embodiments of the isolated oligonucleotide, the isolated oligonucleotide is capable of inducing degradation of the human ACVR1C mRNA.

In some embodiments of the isolated oligonucleotide, the sense strand is a single stranded RNA molecule, the anti-sense strand is a single stranded RNA molecule, or both the sense strand and the anti-sense strand are single stranded RNA molecules. Accordingly, in some embodiments, the isolated oligonucleotides of the present disclosure are double-stranded RNA molecules or partially double-stranded RNA molecules.

In some embodiments of the isolated oligonucleotide, the anti-sense strand comprises a 3′ overhang. In some embodiments of the isolated oligonucleotide, the 3′ overhang comprises at least one nucleotide. In some embodiments of the isolated oligonucleotide, the 3′ overhang comprises two nucleotides. In some embodiments of the isolated oligonucleotide, the 3′ overhang comprises any one of thymidine-thymidine (dTdT), Adenine-Adenine (AA), Cysteine-Cysteine (CC), Guanine-Guanine (GG) or Uracil-Uracil (UU).

In some embodiments of the isolated oligonucleotide, the sense strand comprises an RNA sequence of at least 20 nucleotides in length. In some embodiments of the isolated oligonucleotide, the sense strand comprises an RNA sequence of 20 nucleotides in length.

In some embodiments of the isolated oligonucleotide, the anti-sense strand comprises an RNA sequence of at least 22 nucleotides in length. In some embodiments of the isolated oligonucleotide, the anti-sense strand comprises an RNA sequence of 22 nucleotides in length.

In some embodiments of the isolated oligonucleotide, the double stranded region is between 19 and 21 nucleotides in length. In some embodiments of the isolated oligonucleotide, the double stranded region is 20 nucleotides in length.

In some embodiments of the isolated oligonucleotide of the present disclosure, the antisense strand comprises a nucleotide sequence according to any one of: SEQ ID NOs: 2-269. In some embodiments of the isolated oligonucleotide of the present disclosure, the sense strand comprises a nucleotide sequence according to any one of: SEQ ID NOs: 538-805.

In some embodiments of the isolated oligonucleotide, the sense strand comprises at least one targeting ligand. In some embodiments of the isolated oligonucleotide, the antisense strand comprises at least one targeting ligand. In some embodiments of the isolated oligonucleotide, both the sense strand and the antisense strand comprise at least one targeting ligand. In some embodiments, the targeting ligand comprises an adipose-targeting ligand. In some embodiments, the adipose-targeting ligand is selected from the group consisting of small molecule, peptide, antibody, and carbohydrate. In some embodiments the targeting ligand comprises a lipid.

### BRIEF DESCRIPTION OF THE DRAWINGS

FIGS. 1A-1E are graphs showing percentage (%) of ACVR1C mRNA remaining in non-human primates (cynomolgus monkeys) administered a single dose of exemplary compounds Duplex 537 (FIG. 1A), Duplex 538 (FIG. 1B), Duplex 539 (FIG. 1C), Duplex 540 (FIG. 1D), and Duplex 541 (FIG. 1E) at 3 mg/kg pre-dose (Week −2), and 2, 4, 8, and 12 weeks after dose. Data is represented in Mean+/−SD.

FIG. 2 is a graph showing percentage (%) of ACVR1C mRNA remaining in non-human primates (cynomolgus monkeys) administered a single dose of exemplary compounds Duplex 540 and Duplex 542 at 0.75 mg/kg pre-dose (Day −14), and 14, 28, 56, and 84 days after dose. Data is represented in Mean+/−SD.

### DETAILED DESCRIPTION

The present disclosure provides isolated oligonucleotides (oligonucleotide(s)) that form a double stranded region, preferably small interfering RNAs (siRNAs), that can decrease ACVR1C mRNA expression, in turn leading to a decrease in the degree of ACVR1C protein (also known as activin receptor-like kinase 7 or ALK7) expression in target cells. The oligonucleotides disclosed herein can have therapeutic applications in regulating the expression of ACVR1C for the prevention and/or treatment of a disease or disorder involving ACVR1C such as, but not limited to, metabolic disease including obesity, diabetes and metabolic syndromes, cancer, ocular disease (including extraocular retinoblastoma), acquired hyperkeratosis, bone disorders including fibrodysplasia ossificans progressiva and other skeletal abnormalities, and any other disease or disorder that is characterized by increased levels of ACVR1C. Accordingly, the oligonucleotides disclosed herein can have therapeutic applications in regulating the expression of ACVR1C for the treatment of any the diseases or disorders described above or herein.

In some aspects, the present invention provides compositions and methods of treating a subject having a disorder that would benefit from the reduction in ACVR1C expression. In some aspects, the methods disclosed herein prevent at least one symptom in a subject having a disease or disorder that would benefit from reduction in ACVR1C expression.

Without wishing to be bound by theory, the present disclosure has identified specific regions within the ACVR1C mRNA, that provide targets for binding double stranded oligonucleotides, e.g., siRNA, leading to reduction in level of expression of the ACVR1C mRNA.

The ACVR1C mRNA sequence described herein, is an mRNA sequence encoded by a ACVR1C gene according to GenBank Accession No. NM_145259.3:
>NM_145259.3 Homo sapiens activin A receptor type 1C (ACVR1C), transcript variant 1, mRNA,

(SEQ ID NO: 1)
agtggcaggagcgccgcgcaccgccagccgcagggggcgtgggatgggggcggccggggaggggggcgcccacactgactagag
ccaaccgcgcacttcaaaagggtgtcggtgccgcgctcccctcccgcggcccgggaacttcaaagcgggccgtgctgccccggctgcct
cgctctgctctggggcctcgcagccccggcgcggccgcctggtggcgatgacccgggcgctctgctcagcgctccgccaggctctcctg
ctgctcgcagcggccgccgagctctcgccaggactgaagtgtgtatgtcttttgtgtgattcttcaaacttcacctgccaaacagaaggagca
tgttgggcatcagtcatgctaaccaatggaaaagagcaggtgatcaaatcctgtgtctcccttccagaactgaatgctcaagtcttctgtcata
gttccaacaatgttaccaaaaccgaatgctgcttcacagatttttgcaacaacataacactgcaccttccaacagcatcaccaaatgccccaaa
acttggacccatggagctggccatcattattactgtgcctgtttgcctcctgtccatagctgcgatgctgacagtatgggcatgccagggtcga
cagtgctcctacaggaagaaaaagagaccaaatgtggaggaaccactctctgagtgcaatctggtaaatgctggaaaaactctgaaagatc
tgatttatgatgtgaccgcctctggatctggctctggtctacctctgttggttcaaaggacaattgcaaggacgattgtgcttcaggaaatagta
ggaaaaggtagatttggtgaggtgtggcatggaagatggtgtggggaagatgtggctgtgaaaatattctcctccagagatgaaagatcttg
gtttcgtgaggcagaaatttaccagacggtcatgctgcgacatgaaaacatccttggtttcattgctgctgacaacaaagataatggaacttgg
actcaactttggctggtatctgaatatcatgaacagggctccttatatgactatttgaatagaaatatagtgaccgtggctggaatgatcaagct
ggcgctctcaattgctagtggtctggcacaccttcatatggagattgttggtacacaaggtaaacctgctattgctcatcgagacataaaatca
aagaatatcttagtgaaaaagtgtgaaacttgtgccatagcggacttagggttggctgtgaagcatgattcaatactgaacactatcgacatac
ctcagaatcctaaagtgggaaccaagaggtatatggctcctgaaatgcttgatgatacaatgaatgtgaatatctttgagtccttcaaacgagc
tgacatctattctgttggtctggtttactgggaaatagcccggaggtgttcagtcggaggaattgttgaggagtaccaattgccttattatgacat
ggtgccttcagatccctcgatagaggaaatgagaaaggttgtttgtgaccagaagtttcgaccaagtatcccaaaccagtggcaaagttgtg
aagcactccgagtcatggggagaataatgcgtgagtgttggtatgccaacggagcggcccgcctaactgctcttcgtattaagaagactata
tctcaactttgtgtcaaagaagactgcaaagcctaatgatgataattatgttaaaaagaaatctctcatagctttcttttccattttcccctttatgtga
atgtttttgccattttttttttgttctacctcaaagataagacagtacagtatttaagtgcccataaggcagcatgaaaagataactctaaagttaag
catgggcaggagttgacttcatccaatctctatgttatgtttaattttattttgaaagcaacacctcaactcatctttttatttaataaggaagaaatat
attacaaaagtataaaataagctctataaaaatgttatagtcattaagtttttattttacttgaaccaagagcacatgaatgaacaggaaaagatgt
aaaaacatttttttctgagatgaaaacatattaattaaacatgcaaattagagcatgctatctttaggtgatgcaatctatgtttccccctttttaagtt
agcaggactttttaaaaataaatattgctctaaactttaatatatcgaacgtgagagtggagctgcttagtggaagatgtaagtgaggtgggtgt
cccatgtgcttggtctccccttctgctgttctcctgttcttcataatccactactgcagcagtccctgaaccactaaacttgttcctttcatttacaaa
agagatacctgacatcctgagacactgagaaatgtcctgaagtcacacagctaatggcagaactggcactaggtccaaatcttgtgataatg
aacaccgtaaggttagctagcttcctactttcccttgaatagtgcttttctccctatgtaatatcttttattatgatatttgtggtttagaaggcatattg
agttattttgcagaatcataatggacccgcacaaaatctcagaaccatatctgttgacattttttctcatagaaatatcatggttaccccatttgtta
atgagcattaatgttttctgaacacttccaaagattaatcaaacataaatattcattgtctgaaaatgtctttaagatacaattcagaggtccctattt
cctttgtacatacacacttagaaagaaaagacagaaaaggaagaggaaggaaggaaatattttgagaatatattgagaagaattaagaaaac
tcttcaatgaagtgttaacaaccaaaccctacagacggtatcagaaacagcaaatagatattcctctaccctttcacagtgagtgagtgagtac
agaagaatgctcatgatagttttgccttcattctactttctgtggacacagagtaatgaatatttaatgggacattaaatatgcccttcaaatctata
attttactttggtaaacgagatttaacatgatgtcttttatgctcctaaaacatcttttttcaaactccattccttagaacattcttctactgagatgatc
caagaccaaaagtgttctttggtacttgcttataaagtgatagtacatgttagcatataatgtattttgaagagtgaagtaaatgctattgataaca
gtaaaaaaaaaaaaaaaaactaataacagtaaagaaatgctacttgatttttttttaaactggattgctcagattacctgatcgtggtggaaccct
tttattaagaggaggggaaactttttactatcccatatttaactgttctataaagcaaagcacagcttgggtaatagcgttctgaaggatatacttc
tgtattttctcatagagtacaatttagtgattatgcttcatttcactatggaaatatgttactgaatctatcttcattttactgagttgaaataaggaagg
caaaaaactgacagctatggagtttgcgtgtacttccatactcgttaatgctctcatccacttattaaataatcatagagcacccatatcttgcttg
ccacaatatcgggtacaagagggaatacaaagatagataggtcctgccctcagggattttaaagtctaatttgggaatgggaatagggatgt
gagtgtgtgggggaagagattaaattgacagataaaatacgaagtgggatgtcttgagttctgcatgacagtgggtttctaggataggtctga
aaattgctttcatttgcaacacatttagaaagtagctttatttggatattacagacaatctaaatatatcatcagtttttaaaagtgcctatgtgaagt
gatttttaaaaagagcctatgtgaaggggtaatcttgcttgttcttgttactaatttctcatagattgtttttctgcatatataagaacgaaattatttatt
tactatggttgtacgtgcctcaaataaacaagaatgatatttcctgttttatttacttatgttgggtaaatatgcttattgaatttttaagagaggattttt
taccatctccatttttcttgtcattatgttttgtagcttatttgagggtgtctaaatataatttcatattttattggttcaactttcactctgaagaaatccgt
atgttagtacattttgaggtatttttcttgttcttgtgttgtttaactatgactcctaactgagtagtcttatatttcaattacaaaatacattttttaagaaa
gggaatagagcagcaaaaatgataaggaaaatgttaaaagttgtaatatttcctttactcttaacaggattatatatagaacatgctcacttacaa
aaataggatgatgaagtttagagcataaggcaggcttcttgtatatacttatgtgtcaaatgttatattgtttttaatggagtcccattgtgtaatat
ttatttcttttacattttgttataagcaaaaaaaaaaaaaattctccttaggttatgttcagagtatcagtgttctttactccttacagatattttggctttg
gggtataatacaagacttgggaaaacactattatgaattttcagtactgtataaagtggtgatgggatttaaatgcagcatcactttctgaaaata
agagaaacattatttgttgtcagtatttcagcatgaacttgttgccttgtaaattttgcctttaagtttgtaattggtacagattctgttgtatgctttctt
ctatgtctaaaatatttggcatgtcacatctagaattcttaatttatgttctgacttgagagttaagtgaaacatgactgtcgtgcactattttaggca
tagcacttgcttttcatctttatactttcaattaactttgcattttaaatttccatgattgtatgaaaatagtaacctggttgcagtatctgaaaaaattaa
aattagcttttatttaaaaatgaaaatctacaatagattcattaggttagaagttccagaataatttattattttattacacctactattgtagaattact
aataaaacaaaacaaaactactcctattttcctggaattttgccaccatgtgacttattggggcagagaaaactcagggttgtctttgagtctgca
caaaagcaccagggaacctgcttagcaaatcgtctgaaaacagggagctgatgtttgccattatacaaagtttgagtaaacaacttaaaattg
cttgttagggcatagtctttgattgaaataagtatgagaatgtatttggctaaataaatgtatttaaaatatacaaattttatttcccactggaaaatta
agaaagtagcagtaccaaatgaataaaagctggcagttgatgtcttcaataatcattcctttaaaataaattcacaaacacatcattacaagcta
cttagaaatgtttagtattcgtatcttaaaatggcaccattgtggttttcaaagatattttaaacatcttttgtgaaacatactatttctgtttgacaatg
ctttgttctagctctgtgtgctgatacctacatggagtttttctgctattttagtgaaaacagtaattcttttatcttgagagctgcttctaaataacaaa
aaagttaattggaatgtaagtttttaaaaaatgttaatattaaatagaatttttataatatgggcatttttcaaaacattttaatgaaaatatatagatatt
tgactttctttttttcatctagccttgctttatatttcattatttttctcactttttttcttaatattccctcactatctttcaacattatcattagttcct
aattttaaaaataactcttatttaacttaaccgtctggaatttagccttctaatgaaagagaatcccttagtactctcagagattatataaagttattccaa
ttttgtgtagatgacgaaaccaaggatcagagattaaatgactactagttactagcagaactcttatgaaagcctggtggtgacacccagcactgtt
ccctgccatatccccaacttttactgattaaaaattaattcgcatgcatctaaacctaccttgaatgcacactaacgtaatgtgccattcaatgatg
atgataaaatcccacttttctttgggtttctactaaaataatttactcactcagagtgaggtcaatgagaaaaactaaactaggctaagaaagagt
tgtagaatggttgttgagctaagtaggcaaccactggggtgctgataaaattaatggataaaatttaggaactgtgagagtatagaatttcttag
tgcaagtaatgattagagaagcttcctgacatcccccaccctttctgtaaggactgttctttctctttgtaccttggaatgggggtgaaaggtgat
ttgatagctgaatttgggactatgtccagtgggatattatctaacttttctctctttctcttttttttcccctcagtttctcattagtttgtctttggcattca
tcttcttttagtgcattaaaaaatgtttggcagatctcatcaatcccaagtcactctataattcctgtatttctttagttgtcgtttaacctgtccaaactt
ctacacaatgaacttcttaacaagattttaagttctctgtataagaggtttcacctcataacttctccagtaatccttcatttggcactatagagtattt
gttaatggcagagatgatttttcttttaaaacctaaaaagactagctgttatttgtattctagcttttagctaacatataaagaatgtctacttttgcttt
aatgctaaattccgcttgagaaatagtaactgggaaagacaatttgaaatatatgccccataatgtgattgttaaatttatttctgctgttccatacc
attgctttgttttgcttttgacaaactaagccattatgctattagtttggaatataatactacagcaaaattaggtaacaatccctattttaaaattccc
ctaacaataatagaactgccagcatacttttctctttcagttgtagatgaatacattcgagagaatatgagctgtatttcatcctagattttaatatttt
cagatgtgactgtatttcctgatcattggtccaagttgtcctaaaagaaatttttctctccagacctaacagttttaactgcaagagtttactgtggg
ttatgttaatctgaattttaatagggccactaagaatctgagtgccttaggagattacccttatacccactgccatcacatccagtcaggcctgtt
gtgctctatataaatcttcccagctgaggggcaggtgcgggctaaaatccaactgcaattggctcccagacataattttatattttacagagaa
gcatcttattggcttatatgtgtttaaagaatggtctggcttatacatcttcagaaaatgagaattaaaaagtcaaaataattcttgacatctacaga
ttgaacaaagaacttagaagaaataatactttatcttttcatcctggcattcctgagagaagagaaattgattgtttatcatgttggtttaatttttcaa
cccagacaatctgcagcaaggcacatggaccccaattttgatatcgtccatacagttttcattctatgcatggagctaattactgactttgcctgt
aaagagaggattgtgtgcctaaattttgtctaacaaatgcaagcgtagaatgacatttactaatatttctatttcttccataggctaaataatagtaa
ctaagtatttttaaggacacagccctttttttctctttatacaaaatgagagtatctgagccaaaatattaaattctagttcttttccgcaatgactagt
gtcaagctcatgtactcttctgattctagactggagaagattattcaaacttgatctgtgtttcaggtttttaaatgtcctaaaaacagaaaattaga
ttcagatctcaaaaaaggaattttggattgactttcaaagtactaatactaattatacttttttttggtagcgtgactcttcttatacctaagaacata
ttacaaatgtcaaaaccattgcattttgacattgcaaaacatgccttgaactcttgaactactgtgaaaagaatcaccgttgtaaagactttttgta
agctagctgatactcttaagtatgtaaaaagattgtctttcagccgacaggcccaaaggaatgtatataaggaaggaatatgaaaaaataaatt
aggttttaaaataggaattgggcaataaactgtatcaaaaatatgtagatggattttagtagttgtaatttaaatgtggaaggtgaagagaatttc
aaactccaaagagaaatgaatgatattcagatgtttcattaatttctagtctgtgaaaatatgcattttatagtaatatgtatagacttattttatttaga
aataatagtgttttagaatttattaaaaactcagtgatagcctttataccaaaatgtttaactttaccaacagcaagtcataaaagtatttattttaaa
gctttttaatattatcgtgtaactttcatctgtcttcagatgtaaataattatctgcctaaatgttatatttttatgtatgcattttctgaaaatgtattgtttt
gtaaagtgggaaagataataaatcaagcacttcttgcacttgtttctgtgaagcatatagaactctattttaaataaaggaagatgtgtcgta.

The present disclosure provides an isolated oligonucleotide comprising a sense strand and an antisense strand, wherein the antisense strand is substantially complementary to its corresponding sense strand such that the sense strand and the antisense strand form a double stranded region. In some embodiments, the double stranded region comprises an antisense strand selected from SEQ ID NOs: 2-269 and its corresponding sense strand selected from SEQ ID NOs: 538-805, as set forth in Table 5. As an example of the reference to Table 5, the double stranded region comprises an antisense strand of nucleic acid sequence according to SEQ ID NO: 2 (5′ UUUUUCACAGCCACAUCUUCCC 3′), and a corresponding sense strand of nucleic acid sequence according to SEQ ID NO: 538 (5′ GAAGAUGUGGCUGUGAAAAA 3′), as set forth in Table 5. In addition, SEQ ID NO: 2 and SEQ ID NO: 538 are identified by duplex number 1 (Duplex No.), as set forth in Table 5.

The present disclosure also provides an isolated oligonucleotide comprising a sense strand and an antisense strand, wherein the sense strand or the antisense strand or both comprise one or more modified nucleotide(s). In some embodiments, the antisense strand comprises a modified sequence selected from SEQ ID NOs: 270-537 and its corresponding sense strand selected from SEQ ID NOs: 806-1073, as set forth in Table 6.

The ACVR1C mRNA sequence according to SEQ ID NO: 1, as described herein, is any heterologous mRNA sequence with sufficient identity to a ACVR1C according to Accession No. NM_145259.3, as described herein, that allows binding to the antisense strand of the oligonucleotides of the present disclosure.

In some embodiments of the isolated oligonucleotide of the present disclosure, the isolated oligonucleotide is capable of inducing degradation of the ACVR1C mRNA.

In some embodiments of the isolated oligonucleotide of the present disclosure, the sense strand is a single stranded RNA molecule. In some embodiments of the isolated oligonucleotide of the present disclosure, the antisense strand is a single stranded RNA molecule. In some embodiments of the isolated oligonucleotide of the present disclosure, both the sense strand and the antisense strand are single stranded RNA molecules.

In some embodiments, the isolated oligonucleotide of the present disclosure is a small interfering RNA (siRNA). Accordingly, the disclosure provides siRNAs, wherein the siRNA comprises a sense region and antisense region complementary to the sense region that together form an RNA duplex, and wherein the sense region comprises a sequence at least 70% to 100% identical to a ACVR1C mRNA sequence.

### Definitions

“RNAi” or “RNA interference” refers to the process of sequence-specific post-transcriptional gene silencing, mediated by double-stranded RNA (dsRNA). Duplex RNA siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information, etc. The antisense strand of these molecules can include RNA, DNA, PNA (Peptide Nucleic Acid), or a combination thereof. These DNA/RNA chimera polynucleotide includes, but is not limited to, a double-strand polynucleotide composed of DNA and RNA that inhibits the expression of a target gene. These dsRNA molecules can also include one or more modified nucleotides, as described herein, which can be incorporated on either strand.

In the RNAi gene silencing or knockdown process, dsRNA comprising a first (antisense) strand that is complementary to a portion of a target gene and a second (sense) strand that is fully or partially complementary to the first antisense strand is introduced into an organism. After introduction into the organism, the target gene-specific dsRNA is processed into relatively small fragments (siRNAs) and can subsequently become distributed throughout the organism, decrease messenger RNA of target gene, leading to a phenotype that may come to closely resemble the phenotype arising from a complete or partial deletion of the target gene.

Certain dsRNAs in cells can undergo the action of Dicer enzyme, a ribonuclease III enzyme. Dicer can process the dsRNA into shorter pieces of dsRNA, i.e. siRNAs. RNAi also involves an endonuclease complex known as the RNA induced silencing complex (RISC). Following cleavage by Dicer, siRNAs enter the RISC complex and direct cleavage of a single stranded RNA target having a sequence complementary to the antisense strand of the siRNA duplex. The other strand of the siRNA is the passenger strand. Cleavage of the target RNA takes place in the middle of the region complementary to the antisense strand of the siRNA duplex. siRNAs can thus down regulate or knock down gene expression by mediating RNA interference in a sequence-specific manner.

Alternatively, short oligonucleotides such as siRNA can be specifically delivered into a cell, a tissue, or an organ. Once introduced into the cell, tissue, or organ, the short oligonucleotides are recognized by and loaded into RISC to cleave a target RNA.

As used herein, “target gene” or “target sequence” refers to a gene or gene sequence whose corresponding RNA is targeted for degradation through the RNAi pathway using dsRNAs or siRNAs as described herein. To target a gene, for example using an siRNA, the siRNA comprises an antisense region complementary to, or substantially complementary to, at least a portion of the target gene or sequence, and sense strand complementary to the antisense strand. Once introduced into a cell, the siRNA directs the RISC complex to cleave an RNA comprising a target sequence, thereby degrading the RNA.

As used herein, “oligonucleotide”, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present disclosure further provides a nucleic acid that is the complement (which can be either a full complement or a partial complement) of a nucleic acid, nucleotide sequence, or polynucleotide of this disclosure. When dsRNA is produced synthetically, less common bases, such as inosine, 5-methylcytosine, 6-methyladenine, hypoxanthine and others can also be used for antisense, dsRNA, and ribozyme pairing. Other modifications, such as modification to the phosphodiester backbone, or the 2′-fluoro, the 2′-hydroxy or 2′O-methyl in the ribose sugar group of the RNA can also be made.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “region” or “fragment” is used interchangeably and as applied to an oligonucleotide.

The ACVR1C mRNA sequence, as described herein, will be understood to mean a full length ACVR1C mRNA nucleotide sequence, unless indicated otherwise. The ACVR1C mRNA sequence encodes (i.e., expresses) ACVR1C protein (also known as ALK7 protein). As such, “ACVR1C” may be used interchangeably with “ALK7”. In some embodiments, the ACVR1C mRNA sequence can be a nucleotide sequence of reduced length relative to a reference nucleic acid or nucleotide sequence of the ACVR1C mRNA sequence comprising, consisting essentially of, and/or consisting of a nucleotide sequence of contiguous nucleotides identical or almost identical (e.g., 60%, 70%, 80%, 90%, 92%, 95%, 98% or 99% identical) to the reference nucleic acid or nucleotide sequence. Such a nucleic acid fragment according to the disclosure may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the disclosure.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the term “substantially complementary” is at least 90% (e.g., 91, 92, 93, 94, 95, 96, 97, 98 or 99%) complementary to the sense strand that is substantially identical to the nucleotide sequence within the defined regions in SEQ ID NO: 1. As used herein, the term “substantially complementary” means that two nucleic acid sequences are complementary at least at about 90%, 95% or 99% of their nucleotides.

In some embodiments, the two nucleic acid sequences can be complementary at least at 90%, 95%, 96%, 97%, 98%, 99% or more of their nucleotides. In some embodiments, the two nucleic acid sequences can be between 90% to 95% complementary, between 70% to 100% complementary, between 95% and 96% complementary, between 90% and 100% complementary, between 96% to 97% complementary, between 60% to 80% complementary, between 97% and 98% complementary, between 70% and 90% complementary, between 98% and 99% complementary, between 80% and 100% complementary, or between 99% and 100% complementary.

The term “substantially complementary” can also mean that two nucleic acid sequences, sense strand and antisense strand have sufficient complementarity that allows binding between the sense strand and antisense strand to form a double stranded region comprising of between 19-25 nucleotides in length. The term “substantially complementary” can also mean that two nucleic acid sequences can hybridize under high stringency conditions, and such conditions are well known in the art.

As used herein, the term “substantially identical” or “sufficient identity” used interchangeably herein, is at least 70%, at least 80%, at least 90%, at least 95%, or at least 99% (e.g., between 70% to 805, 8-% to 90% or 90% to 95% or 95% to 99% or 99% to 100%) identical to the nucleotide sequence within the defined regions in SEQ ID NO: 1.

As used herein, the term “identity” means that sequences are compared with one another as follows. In order to determine the percentage identity of two nucleic acid sequences, the sequences can first be aligned with respect to one another in order subsequently to make a comparison of these sequences possible. For this e.g., gaps can be inserted into the sequence of the first nucleic acid sequence and the nucleotides can be compared with the corresponding position of the second nucleic acid sequence. If a position in the first nucleic acid sequence is occupied by the same nucleotide as is the case at a position in the second sequence, the two sequences are identical at this position. The percentage identity between two sequences is a function of the number of identical positions divided by the number of all the positions compared in the sequences investigated.

A “percent identity” or “% identity” as used interchangeably herein, for aligned segments of a test sequence and a reference sequence is the percent of identical components which are shared by the two aligned sequences divided by the total number of components in reference sequence segment, i.e., the entire reference sequence or a smaller defined part of the reference sequence.

“Nucleotide sequence” and “nucleic acid sequence” are used interchangeably herein, unless indicated otherwise.

The percentage identity of two sequences can be determined with the aid of a mathematical algorithm. A preferred, but not limiting, example of a mathematical algorithm which can be used for comparison of two sequences is the algorithm of Karlin et al. (1993), PNAS USA, 90:5873-5877. Such an algorithm is integrated in the NBLAST program, with which sequences which have a desired identity to the sequences of the present disclosure can be identified. In order to obtain a gapped alignment, as described here, the “Gapped BLAST” program can be used, as is described in Altschul et al. (1997), Nucleic Acids Res, 25:3389-3402. If BLAST and Gapped BLAST programs are used, the preset parameters of the particular program (e.g. NBLAST) can be used. The sequences can be aligned further using version 9 of GAP (global alignment program) of the “Genetic Computing Group” using the preset (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of agreements as a percentage content of the nucleic acids in the sequence claimed. The methods described for determination of the percentage identity of two nucleic acid sequences can also be used correspondingly, if necessary, on the coded amino acid sequences.

Useful methods for determining sequence identity are also disclosed in Guide to Huge Computers (Martin J. Bishop, ed., Academic Press, San Diego (1994)), and Carillo, H., and Lipton, D., (Applied Math48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Percent identity can be 70% identity or greater, e.g., at least 70% identity, at least 75% identity, at least 80% identity, at least 85% identity, at least 90% identity, at least 95% identity, at least 98% identity, at least 99% identity or 100% identity.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

As used herein, “hydrocarbon chain” refers to a linear or branched moiety consisting of hydrogen and carbon, except for otherwise specified one or more substitutions. In some embodiments, the hydrocarbon chain is a saturated hydrocarbon chain (e.g., alkyl). In some embodiments, the hydrocarbon chain is an unsaturated hydrocarbon chain (e.g., containing one or more double bonds and/or one or more triple bonds (e.g., alkenyl or alkynyl)). In some embodiments, two substituents of the hydrocarbon chain (e.g., two R^{L′}), together with the one or more intervening atoms, form a ring moiety (e.g., C₃-C₈ cycloalkyl or 3- to 8-membered heterocycloalkyl), wherein the substituents comprise at least one ring atom. When the ring moiety is at an internal position of the hydrocarbon chain, the carbon atoms of the hydrocarbon chain (e.g., C₂-C₃₅) are counted via the shortest path through the ring (e.g.,

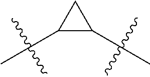 [SMILES: *C1CC1*]

comprises two carbon atoms of the hydrocarbon chain, wherein each [symbol] indicates a point of attachment to the rest of the hydrocarbon chain). When the ring moiety is at a terminal position of the hydrocarbon chain, the carbon atoms of the hydrocarbon chain (e.g., C₂-C₃₅) are counted via the longest path within the ring (e.g.,

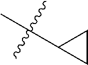 [SMILES: *C1CC1]

comprises two carbon atoms of the hydrocarbon chain, wherein [symbol] indicates the point of attachment to the rest of the hydrocarbon chain).

As used herein, “hetero-hydrocarbon chain” refers to a linear or branched moiety consisting of hydrogen, carbon, and one or more heteroatoms (such as O, N, S, P, or Se), e.g., 1 or 1-2 or 1-3 or 1-4 or 1-5 or 1-6 or 1-7 or 1-8 or 1-9 or 1-10 or 1-11 or 1-12 or 1-13 or 1-14 heteroatoms, or e.g. 1, 2, 3, 4, 5, 6, 7, 8, 9, 10, 11, 12, 13, or 14 heteroatoms, except for otherwise specified one or more substitutions. It is understood that an “X-membered” hetero-hydrocarbon chain refers to a hetero-hydrocarbon chain where the total atom count of carbon and heteroatoms along the longest path of the chain is “X”, (e.g., 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32- , 33-, 34- or 35-membered hetero-hydrocarbon chain), except for otherwise specified one or more substitutions. In some embodiments, the hetero-hydrocarbon chain is a saturated hetero-hydrocarbon chain. In some embodiments, the hetero-hydrocarbon chain is an unsaturated hetero-hydrocarbon chain (e.g., containing one or more double bonds and/or one or more triple bonds). In some embodiments, two substituents of the hetero-hydrocarbon chain (e.g., two R^{L′}), together with the one or more intervening atoms, form a ring moiety (e.g., C₃-C₈ cycloalkyl or 3- to 8-membered heterocycloalkyl), wherein the substituents comprise at least one ring atom. When the ring moiety is at an internal position of the hetero-hydrocarbon chain, the atoms of the hetero-hydrocarbon chain (e.g., 2-35 members) are counted via the shortest path through the ring (e.g.,

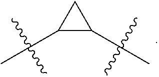 [SMILES: *C1CC1*]

comprises two atoms of the hetero-hydrocarbon chain, wherein each [symbol] indicates a point of attachment to the rest of the hetero-hydrocarbon chain). When the ring moiety is at a terminal position of the hetero-hydrocarbon chain, the atoms of the hetero-hydrocarbon chain (e.g., 2-35 members) are counted via the longest path within the ring (e.g.,

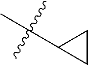 [SMILES: *C1CC1]

comprises two atoms of the hetero-hydrocarbon chain, wherein [symbol] indicates the point of attachment to the rest of the hetero-hydrocarbon chain).

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, i-pentyl, or n-hexyl. In some embodiments, a straight chain or branched alkyl has six or fewer carbon atoms (e.g., C₁-C₆ for straight chain, C₃-C₆ for branched chain), and in another embodiment, a straight chain or branched alkyl has four or fewer carbon atoms.

As used herein, the term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkylene” refers to a divalent group resulting from the removal of a hydrogen radical from an alkyl group.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In some embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

As used herein, the term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In some embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms. As used herein, “C₂-C₆ alkenylene linker” or “C₂-C₆ alkynylene linker” is intended to include C₂, C₃, C₄, C₅ or C₆ chain (linear or branched) divalent unsaturated aliphatic hydrocarbon groups. For example, C₂-C₆ alkenylene linker is intended to include C₂, C₃, C₄, C₅ and C₆ alkenylene linker groups.

As used herein, the term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, the term “cycloalkyl” refers to a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, 1,2,3,4-tetrahydronaphthalenyl, and adamantyl. In the case of polycyclic cycloalkyl, only one of the rings in the cycloalkyl needs to be non-aromatic.

As used herein, the term “heterocycloalkyl” refers to a saturated or partially unsaturated 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, P, or Se), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, 5,6-dihydro-4H-cyclopenta[b]thiophenyl, and the like. In the case of multicyclic heterocycloalkyl, only one of the rings in the heterocycloalkyl needs to be non-aromatic (e.g., 4,5,6,7-tetrahydrobenzo[c]isoxazolyl).

As used herein, the term “aryl” includes groups with aromaticity, including “conjugated,” or multicyclic systems with one or more aromatic rings and do not contain any heteroatom in the ring structure. The term aryl includes both monovalent species and divalent species. Examples of aryl groups include, but are not limited to, phenyl, biphenyl, naphthyl and the like. Conveniently, an aryl is phenyl.

As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like. Heteroaryl groups can also be fused or bridged with alicyclic or heterocyclic rings, which are not aromatic so as to form a multicyclic system (e.g., 4,5,6,7-tetrahydrobenzo[c]isoxazolyl). In some embodiments, the heteroaryl is thiophenyl or benzothiophenyl. In some embodiments, the heteroaryl is thiophenyl. In some embodiments, the heteroaryl benzothiophenyl.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, quinoline, isoquinoline, naphthrydine, indole, benzofuran, purine, benzofuran, deazapurine, indolizine.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl such as benzo[d][1,3]dioxole-5-yl).

As used herein, the term “substituted,” means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

As used herein, the term “vicinal” describes the relationship between two moieties bonded to two adjacent carbon atoms.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R) occurs more than one time in any constituent or formula for a compound, its definition at each occurrence is independent of its definition at every other occurrence. Thus, for example, if a group is shown to be substituted with 0-2 R moieties, then the group may optionally be substituted with up to two R moieties and R at each occurrence is selected independently from the definition of R. Also, combinations of substituents and/or variables are permissible, but only if such combinations result in stable compounds.

As used herein, the term “hydroxy” or “hydroxyl” includes groups with an —OH or —O⁻.

As used herein, the term “halo” or “halogen” refers to fluoro, chloro, bromo and iodo.

The term “haloalkyl” or “haloalkoxyl” refers to an alkyl or alkoxyl substituted with one or more halogen atoms.

As used herein, the term “optionally substituted haloalkyl” refers to unsubstituted haloalkyl or substituted haloalkyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkoxy” or “alkoxyl” includes substituted and unsubstituted alkyl, alkenyl and alkynyl groups covalently attached to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

As used herein, the term “saccharide moiety” refers to a monovalent group resulting from the removal of a hydrogen or hydroxyl from a monosaccharide or polysaccharide (e.g., a monosaccharide or polysaccharide of mannose, or a monosaccharide or polysaccharide of fucose), and wherein one or more hydroxyl of the monosaccharide or polysaccharide may be independently replaced by hydrogen, halogen, or C₁-C₆ alkyl optionally substituted with one or more halogen.

As used herein, the term “monosaccharide moiety” refers to a monovalent group resulting from the removal of a hydrogen or hydroxyl from a monosaccharide (e.g., a monosaccharide of mannose or fucose), and wherein one or more hydroxyl of the monosaccharide may be independently replaced by hydrogen, halogen, or C₁-C₆ alkyl optionally substituted with one or more halogen.

As used herein, the term “disaccharide moiety” refers to a monovalent group resulting from the removal of a hydrogen or hydroxyl from a disaccharide (e.g., a disaccharide of mannose or fucose), and wherein one or more hydroxyl of the disaccharide may be independently replaced by hydrogen, halogen, or C₁-C₆ alkyl optionally substituted with one or more halogen.

As used herein, the term “polysaccharide moiety” refers to a monovalent group resulting from the removal of a hydrogen or hydroxyl from a polysaccharide (e.g., a polysaccharide comprising two or more mannose units or a polysaccharide comprising two or more fucose units), and wherein one or more hydroxyl of the polysaccharide may be independently replaced by hydrogen, halogen, or C₁-C₆ alkyl optionally substituted with one or more halogen.

As used herein, the term “mannose moiety” refers to a monovalent group resulting from the removal of a hydrogen or hydroxyl from a monosaccharide of mannose or a polysaccharide comprising two or more mannose, and wherein one or more hydroxyl of the monosaccharide or polysaccharide may be independently replaced by hydrogen, halogen, or C₁-C₆ alkyl optionally substituted with one or more halogen.

As used herein, the term “monomannose moiety” refers to a monovalent group resulting from the removal of a hydrogen or hydroxyl from a monomannose (i.e., a monosaccharide of mannose), and wherein one or more hydroxyl of the monomannose may be independently replaced by hydrogen, halogen, or C₁-C₆ alkyl optionally substituted with one or more halogen.

As used herein, the term “dimannose moiety” refers to a monovalent group resulting from the removal of a hydrogen or hydroxyl from a dimannose (i.e., a disaccharide of mannose), and wherein one or more hydroxyl of the dimannose may be independently replaced by hydrogen, halogen, or C₁-C₆ alkyl optionally substituted with one or more halogen.

As used herein, the term “polymannose moiety” refers to a monovalent group resulting from the removal of a hydrogen or hydroxyl from a polymannose (i.e., a polysaccharide comprising two or more mannose units), and wherein one or more hydroxyl of the polymannose may be independently replaced by hydrogen, halogen, or C₁-C₆ alkyl optionally substituted with one or more halogen.

As used herein, the term “fucose moiety” refers to a monovalent group resulting from the removal of a hydrogen or hydroxyl from a monosaccharide of fucose or a polysaccharide comprising two or more fucose, and wherein one or more hydroxyl of the monosaccharide or polysaccharide may be independently replaced by hydrogen, halogen, or C₁-C₆ alkyl optionally substituted with one or more halogen.

As used herein, the term “monofucose moiety” refers to a monovalent group resulting from the removal of a hydrogen or hydroxyl from a monofucose (i.e., a monosaccharide of fucose), and wherein one or more hydroxyl of the monofucose may be independently replaced by hydrogen, halogen, or C₁-C₆ alkyl optionally substituted with one or more halogen.

As used herein, the term “difucose moiety” refers to a monovalent group resulting from the removal of a hydrogen or hydroxyl from a difucose (i.e., a disaccharide of fucose), and wherein one or more hydroxyl of the difucose may be independently replaced by hydrogen, halogen, or C₁-C₆ alkyl optionally substituted with one or more halogen.

As used herein, the term “polyfucose moiety” refers to a monovalent group resulting from the removal of a hydrogen or hydroxyl from a polyfucose (i.e., a polysaccharide comprising two or more fucose units), and wherein one or more hydroxyl of the polyfucose may be independently replaced by hydrogen, halogen, or C₁-C₆ alkyl optionally substituted with one or more halogen.

The disclosure provides isolated oligonucleotides comprising a double stranded RNAs (dsRNAs) duplex region which target ACVR1C mRNA sequence for degradation. The double stranded RNA molecule of the disclosure may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the dsRNA is a small interfering RNA (siRNA) which targets a ACVR1C mRNA sequence for degradation. In some embodiments, the siRNA targeting ACVR1C is packaged in a delivery system described herein (e.g., nanoparticle).

The isolated oligonucleotides of the present disclosure targeting ACVR1C for degradation can comprise a sense strand at least 70% identical to any fragment of a ACVR1C mRNA, for example the ACVR1C mRNA of SEQ ID NO: 1. In some embodiments, the sense strand comprises or consists essentially of a sequence at least 70%, at least 80%, at least 90%, at least 95% or is 100% identical to any fragment of SEQ ID NO: 1. The siRNAs targeting ACVR1C for degradation can comprise an antisense strand at least 70% identical to a sequence complementary to any fragment of a ACVR1C mRNA, for example the ACVR1C mRNA of SEQ ID NO: 1. In some embodiments, the antisense strand comprises or consists essentially of a sequence at least 70%, at least 80%, at least 90%, at least 95% or is 100% identical to a sequence complementary to any fragment of SEQ ID NO: 1. In some embodiments, the sense region and antisense regions are complementary, and base pair to form an RNA duplex structure. The fragment of the ACVR1C mRNA that has percent identity to the sense region of the siRNA, and which is complementary to the antisense region of the siRNA, can be protein coding sequence of the mRNA, an untranslated region (UTR) of the mRNA (5′ UTR or 3′ UTR), or both.

In some embodiments, the isolated oligonucleotides of the present disclosure comprises a sense region and antisense region complementary to the sense region that together form an RNA duplex, and the sense region comprises a sequence at least 70% identical to a ACVR1C mRNA sequence. In some embodiments, the sense region is identical to a ACVR1C mRNA sequence.

As used herein, the term “sense strand” or “sense region” refers to a nucleotide sequence of an siRNA molecule that is partially or fully complementary to at least a portion of a corresponding antisense strand or antisense region of the siRNA molecule. The sense strand of an isolated oligonucleotides of the present disclosure molecule can include a nucleic acid sequence having some percentage identity with a target nucleic acid sequence such as a ACVR1C mRNA sequence. In some cases, the sense region may have 100% identity, i.e., complete identity or homology, to the target nucleic acid sequence. In other cases, there may be one or more mismatches between the sense region and the target nucleic acid sequence. For example, there may be 1, 2, 3, 4, 5, 6, or 7 mismatches between the sense region and the target nucleic acid sequence.

As used herein, the term “antisense strand” or “antisense region” refers to a nucleotide sequence of the isolated oligonucleotides of the present disclosure, that is partially or fully complementary to at least a portion of a target nucleic acid sequence. The antisense strand of an isolated oligonucleotides of the present disclosure molecule can include a nucleic acid sequence that is complementary to at least a portion of a corresponding sense strand of the isolated oligonucleotides.

In some embodiments, the sense region comprises a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region consists essentially of a sequence that is at least 70% identical, at least 75% identical, at least 80% identical, at least 85% identical, at least 90% identical, at least 95% identical, at least 97% identical, at least 99% identical or 100% identical to a sequence of SEQ ID NO: 1 or a region of SEQ ID NO: 1, as disclosed herein. In some embodiments, the sense region comprises a sequence that is identical to a sequence of SEQ ID NO: 1 or a region of SEQ ID NO: 1, as disclosed herein. In some embodiments, the sense region consists essentially of a sequence that is identical to a sequence of SEQ ID NO: 1 or a region of SEQ ID NO: 1, as disclosed herein.

In some embodiments, the sense region of the isolated oligonucleotides of the present disclosure targeting ACVR1C has one or more mismatches between the sequence of the isolated oligonucleotides and the ACVR1C sequence. For example, the sequence of the sense region may have 1, 2, 3, 4 or 5 mismatches between the sequence of the sense region of the isolated oligonucleotides and the ACVR1C sequence. In some embodiments, the ACVR1C sequence is an ACVR1C 3′ untranslated region sequence (3′ UTR). Without wishing to be bound by theory, it is thought that siRNAs targeting the 3′ UTR have elevated mismatch tolerance when compared to mismatches in the isolated oligonucleotides targeting coding regions of a gene. Further, the isolated oligonucleotides RNAs may be tolerant of mismatches outside the seed region. As used herein, the “seed region” of the isolated oligonucleotides refers to nucleotides 2-8 from the 5′ end of the antisense strand of the isolated oligonucleotides, i.e., the strand of the isolated oligonucleotides that is complementary to and hybridizes to the target mRNA.

In some embodiments, the antisense region comprises a sequence that is at least 70% identical, at least 75% identical, at least 80% identical, at least 85% identical, at least 90% identical, at least 95% identical, at least 97% identical, at least 99% identical or 100% identical to a sequence complementary to a sequence of SEQ ID NO: 1 or a region of SEQ ID NO: 1, as disclosed herein. In some embodiments, the antisense region consists essentially of a sequence that is at least 70% identical, at least 75% identical, at least 80% identical, at least 85% identical, at least 90% identical, at least 95% identical, at least 97% identical, at least 99% or 100% identical to a sequence complementary to a sequence of SEQ ID NO: 1 or a region of SEQ ID NO: 1. In some embodiments, the antisense region comprises a sequence that is identical to a sequence complementary to a sequence of SEQ ID NO: 1 or a region of SEQ ID NO: 1. In some embodiments, the sense region consists essentially of a sequence that is complementary to a sequence of SEQ ID NO: 1 or a region of SEQ ID NO: 1.

The antisense region of the ACVR1C targeting isolated oligonucleotide of the present disclosure is complementary to the sense region. In some embodiments, the sense region and the antisense region are fully complementary (no mismatches). In some embodiments the antisense region is partially complementary to the sense region, i.e., there are 1, 2, 3, 4 or 5 mismatches between the sense region and the antisense region.

In general, isolated oligonucleotides of the present disclosure comprise an RNA duplex that is about 16 to about 25 nucleotides in length. In some embodiments, the RNA duplex is between about 17 and about 24 nucleotides in length, between about 18 and about 23 nucleotides in length, or between about 19 and about 22 nucleotides in length. In some embodiments, the RNA duplex is 19 nucleotides in length. In some embodiments, the RNA duplex is 20 nucleotides in length.

In some embodiments of the isolated oligonucleotide of the present disclosure, the sense strand is a single stranded RNA molecule. In some embodiments of the isolated oligonucleotide of the present disclosure, the antisense strand is a single stranded RNA molecule. In some embodiments, both the sense strand and the antisense strand are single stranded RNA molecules. In some embodiments, the isolated oligonucleotide of the present disclosure is an siRNA targeting ACVR1C that comprises two different single stranded RNAs, the first comprising the sense region and the second comprising the antisense region, which hybridize to form an RNA duplex.

In some embodiments, the isolated oligonucleotide of the present disclosure can have one or more overhangs from the duplex region. In some embodiments, the overhangs, which are non-base-paired, single strand regions, can be from one to eight nucleotides in length, or longer. In some embodiments, the overhang can be a 3′ overhang, wherein the 3′-end of a strand has a single strand region of from one to eight nucleotides. In some embodiments, the overhang can be a 5′ overhang, wherein the 5′-end of a strand has a single strand region of from one to eight nucleotides. In some embodiments, the overhangs of the isolated oligonucleotide are the same length. In some embodiments, the overhangs of the isolated oligonucleotide are different lengths.

In some embodiments of the isolated oligonucleotide of the present disclosure, the single stranded RNA molecule of the sense strand comprises a 3′ overhang. In some embodiments, the 3′ overhang of the single stranded RNA molecule of the sense strand comprises at least one nucleotide. In some embodiments, the 3′ overhang of the single stranded RNA molecule of the sense strand comprises two nucleotides.

In some embodiments of the isolated oligonucleotide of the present disclosure, the single stranded RNA molecule of the antisense strand comprises a 3′ overhang. In some embodiments, the 3′ overhang of the single stranded RNA molecule of the antisense strand comprises at least one nucleotide. In some embodiments, the 3′ overhang of the single stranded RNA molecule of the antisense strand comprises two nucleotides.

In some embodiments of the isolated oligonucleotide of the present disclosure, both ends of isolated oligonucleotide have an overhang, for example, a 3′ dinucleotide overhang on each end. In some embodiments, the overhangs at the 5′- and 3′-ends are of different lengths. In some embodiments, the overhangs at the 5′- and 3′-ends are of the same length.

In some embodiments of the isolated oligonucleotide of the present disclosure, the overhang can contain one or more deoxyribonucleotides, one or more ribonucleotides, or a combination of deoxyribonucleotides and ribonucleotides. In some embodiments, one, or both, of the overhang nucleotides of an siRNA may be 2′-deoxyribonucleotides.

In some embodiments of the isolated oligonucleotide of the present disclosure, the first single stranded RNA molecule comprises a first 3′ overhang. In some embodiments, the second single stranded RNA molecule comprises a second 3′ overhang. In some embodiments, the first and second 3′ overhangs comprise a dinucleotide.

In some embodiments of the isolated oligonucleotide of the present disclosure, the 3′ overhang comprises any one of thymidine-thymidine (dTdT), Adenine-Adenine (AA), Cysteine-Cysteine (CC), Guanine-Guanine (GG) or Uracil-Uracil (UU). In some embodiments, the isolated oligonucleotide of the present disclosure, the 3′ overhang comprises a thymidine-thymidine (dTdT) or a Uracil-Uracil (UU) overhang. In some embodiments, the 3′ overhang comprises a Uracil-Uracil (UU) overhang. Without wishing to be bound by theory, it is thought that 3′ overhangs, such as dinucleotide overhangs, enhance siRNA mediated mRNA degradation by enhancing siRNA-RISC complex formation, and/or rate of cleavage of the target mRNA by the siRNA-RISC complex.

In some embodiments, the isolated oligonucleotide of the present disclosure can have one or more blunt ends, in which the duplex region ends with no overhang, and the strands are base paired to the end of the duplex region. In some embodiments, the isolated oligonucleotide of the present disclosure can have one or more blunt ends, or can have one or more overhangs, or can have a combination of a blunt end and an overhang end. For example, the 5′ end of the siRNA can be blunt and the 3′ end of the same isolated oligonucleotide comprise an overhang, or vice versa.

In some embodiments, both ends of the isolated oligonucleotide of the present disclosure are blunt ends.

In some embodiments of the isolated oligonucleotide of the present disclosure, the double stranded region comprises an antisense strand and a sense strand, according to any one of the pairs of antisense strand and sense strand sequences in Table 5, as described below.

Accordingly, the isolated oligonucleotides disclosed in the present disclosure are useful in treating or preventing a disease or disorder associated with aberrant or increased expression or activity of ACVR1C or a disease or disorder where ACVR1C plays a role. In some embodiments, the isolated oligonucleotide consists of a sequence disclosed herein. In some embodiments, the isolated oligonucleotide essentially consists of a sequence disclosed herein. In some embodiments, the isolated oligonucleotide consists of a sense strand disclosed herein. In some embodiments, the isolated oligonucleotide consists of an antisense strand disclosed herein. In some embodiments, the isolated oligonucleotide essentially consists of a sense strand disclosed herein. In some embodiments, the isolated oligonucleotide essentially consists of an antisense strand disclosed herein. In some embodiments, the isolated oligonucleotide consists of an antisense strand and its corresponding sense strand disclosed herein. In some embodiments, the isolated oligonucleotide essentially consists of an antisense strand and its corresponding sense strand disclosed herein. Exemplary isolated oligonucleotides of the present disclosure are described in Table 5 and Table 6. In some embodiments of the isolated oligonucleotide of the present disclosure, the sense strand comprises a sequence selected from any one of the groups of sense strand/passenger strand sequences listed in Table 5 or Table 6. In some embodiments, the antisense strand comprises a sequence selected from any one of the groups of antisense strand/guide strand sequences listed in Table 5 and Table 6. In some embodiments, the sense and antisense regions comprise corresponding complementary sequences selected from the group listed in Table 5 and Table 6. In some embodiments, the isolated oligonucleotide consists of a sequence selected from any one of the groups of sense strands listed in Table 5 or Table 6. In some embodiments, the isolated oligonucleotide consists of a sequence selected from any one of the groups of antisense strands listed in Table 5 or Table 6. In some embodiments, the isolated oligonucleotide essentially consists of a sequence selected from any one of the groups of sense strands listed in Table 5 or Table 6. In some embodiments, the isolated oligonucleotide essentially consists of a sequence selected from any one of the groups of antisense strands listed in Table 5 or Table 6.

In some embodiments of the isolated oligonucleotide of the present disclosure, the antisense strand comprises a nucleotide sequence according to any one of: SEQ ID NOs: 2-269. In some embodiments of the isolated oligonucleotide of the present disclosure, the antisense strand consists of a nucleotide sequence according to any one of: SEQ ID NOs: 2-269. In some embodiments of the isolated oligonucleotide of the present disclosure, the antisense strand essentially consists of a nucleotide sequence according to any one of: SEQ ID NOs: 2-269.

In some embodiments of the isolated oligonucleotide of the present disclosure, the sense strand comprises a nucleotide sequence according to any one of: SEQ ID NOs: 538-805. In some embodiments of the isolated oligonucleotide of the present disclosure, the sense strand consists of a nucleotide sequence according to any one of: SEQ ID NOs: 538-805. In some embodiments of the isolated oligonucleotide of the present disclosure, the sense strand essentially consists of a nucleotide sequence according to any one of: SEQ ID NOs: 538-805.

In some embodiments of the isolated oligonucleotide of the present disclosure, the antisense strand comprises a nucleotide sequence according to any one of: SEQ ID NOs: 2-269; and the sense strand comprises a nucleotide sequence according to any one of: SEQ ID NOs: 538-805, wherein the antisense strand and the sense strand sequences have sufficient complementarity to allow formation of a double stranded region between the antisense and the sense strand. In some embodiments of the isolated oligonucleotide of the present disclosure, the antisense strand consists of a nucleotide sequence according to any one of: SEQ ID NOs: 2-269; and the sense strand consists of a nucleotide sequence according to any one of: SEQ ID NOs: 538-805, wherein the antisense strand and the sense strand sequences have sufficient complementarity to allow formation of a double stranded region between the antisense and the sense strand. In some embodiments of the isolated oligonucleotide of the present disclosure, the antisense strand essentially consists of a nucleotide sequence according to any one of: SEQ ID NOs: 2-269; and the sense strand essentially consists of a nucleotide sequence according to any one of: SEQ ID NOs: 538-805, wherein the antisense strand and the sense strand sequences have sufficient complementarity to allow formation of a double stranded region between the antisense and the sense strand.

The present disclosure provides an isolated oligonucleotide comprising a sense strand and an antisense strand, wherein the antisense strand is substantially complementary to the sense strand such that the sense strand and the antisense strand form a double stranded region, wherein the double stranded region comprises an antisense strand selected from SEQ ID NOs: 2-269 and its corresponding sense strand selected from SEQ ID NOs: 538-805, as set forth in Table 5 and Table 6. In some embodiments, the double stranded region consists of an antisense strand selected from SEQ ID NOs: 2-269 and its corresponding sense strand selected from SEQ ID NOs: 538-805, as set forth in Table 5 and Table 6. In some embodiments, the double stranded region essentially consists of an antisense strand selected from SEQ ID NOs: 2-269 and its corresponding sense strand selected from SEQ ID NOs: 538-805, as set forth in Table 5 and Table 6.

In some embodiments, the antisense strand comprises SEQ ID NO: 58 and its corresponding sense strand comprises SEQ ID NO: 594. In some embodiments, the antisense strand consists of SEQ ID NO: 58 and its corresponding sense strand consists of SEQ ID NO: 594. In some embodiments, the antisense strand essentially consists of SEQ ID NO: 58 and its corresponding sense strand essentially consists of SEQ ID NO: 594.

In some embodiments, the antisense strand comprises SEQ ID NO: 95 and its corresponding sense strand comprises SEQ ID NO: 631. In some embodiments, the antisense strand consists of SEQ ID NO: 95 and its corresponding sense strand consists of SEQ ID NO: 631. In some embodiments, the antisense strand essentially consists of SEQ ID NO: 95 and its corresponding sense strand essentially consists of SEQ ID NO: 631.

In some embodiments, the antisense strand comprises SEQ ID NO: 36 and its corresponding sense strand comprises SEQ ID NO: 572. In some embodiments, the antisense strand consists of SEQ ID NO: 36 and its corresponding sense strand consists of SEQ ID NO: 572. In some embodiments, the antisense strand essentially consists of SEQ ID NO: 36 and its corresponding sense strand essentially consists of SEQ ID NO: 572.

In some embodiments, the antisense strand comprises SEQ ID NO: 21 and its corresponding sense strand comprises SEQ ID NO: 557. In some embodiments, the antisense strand consists of SEQ ID NO: 21 and its corresponding sense strand consists of SEQ ID NO: 557. In some embodiments, the antisense strand essentially consists of SEQ ID NO: 21 and its corresponding sense strand essentially consists of SEQ ID NO: 557.

In some embodiments, the antisense strand comprises SEQ ID NO: 61 and its corresponding sense strand comprises SEQ ID NO: 597. In some embodiments, the antisense strand consists of SEQ ID NO: 61 and its corresponding sense strand consists of SEQ ID NO: 597. In some embodiments, the antisense strand essentially consists of SEQ ID NO: 61 and its corresponding sense strand essentially consists of SEQ ID NO: 597.

The present disclosure also provides an isolated oligonucleotide comprising a sense strand and an antisense strand, wherein the sense strand or the antisense strand or both comprise one or more modified nucleotide(s). In some embodiments, the antisense strand comprises a modified sequence selected from SEQ ID NOs: 270-537 and SEQ ID NOs: 1074-1078. In some embodiments, the antisense strand consists of a modified sequence selected from SEQ ID NOs: 270-537 and SEQ ID NOs: 1074-1078. In some embodiments, the antisense strand essentially consists of a modified sequence selected from SEQ ID NOs: 270-537 and SEQ ID NOs: 1074-1078.

In some embodiments, the sense strand comprises a modified sequence selected from SEQ ID NOs: 806-1073 and SEQ ID NOs: 1079-1084. In some embodiments, the sense strand consists of a modified sequence selected from SEQ ID NOs: 806-1073 and SEQ ID NOs: 1079-1084. In some embodiments, the sense strand essentially consists of a modified sequence selected from SEQ ID NOs: 806-1073 and SEQ ID NOs: 1079-1084.

In some embodiments, the antisense strand comprises a modified sequence selected from SEQ ID NOs: 270-537 and SEQ ID NOs: 1074-1078, and its corresponding sense strand comprises a modified sequence selected from SEQ ID NOs: 806-1073 and SEQ ID NOs: 1079-1084, as set forth in Table 6. In some embodiments, the antisense strand consists of a modified sequence selected from SEQ ID NOs: 270-537 and SEQ ID NOs: 1074-1078, and its corresponding sense strand consists of a modified sequence selected from SEQ ID NOs: 806-1073 and SEQ ID NOs: 1079-1084, as set forth in Table 6. In some embodiments, the antisense strand essentially consists of a modified sequence selected from SEQ ID NOs: 270-537 and SEQ ID NOs: 1074-1078, and its corresponding sense strand essentially consists of a modified sequence selected from SEQ ID NOs: 806-1073 and SEQ ID NOs: 1079-1084, as set forth in Table 6.

In some embodiments, the antisense strand comprises SEQ ID NO: 1074 and its corresponding sense strand comprises SEQ ID NO: 1079. In some embodiments, the antisense strand consists of SEQ ID NO: 1074 and its corresponding sense strand consists of SEQ ID NO: 1079. In some embodiments, the antisense strand essentially consists of SEQ ID NO: 1074 and its corresponding sense strand essentially consists of SEQ ID NO: 1079.

In some embodiments, the antisense strand comprises SEQ ID NO: 1075 and its corresponding sense strand comprises SEQ ID NO: 1080. In some embodiments, the antisense strand consists of SEQ ID NO: 1075 and its corresponding sense strand consists of SEQ ID NO: 1080. In some embodiments, the antisense strand essentially consists of SEQ ID NO: 1075 and its corresponding sense strand essentially consists of SEQ ID NO: 1080.

In some embodiments, the antisense strand comprises SEQ ID NO: 1076 and its corresponding sense strand comprises SEQ ID NO: 1081. In some embodiments, the antisense strand consists of SEQ ID NO: 1076 and its corresponding sense strand consists of SEQ ID NO: 1081. In some embodiments, the antisense strand essentially consists of SEQ ID NO: 1076 and its corresponding sense strand essentially consists of SEQ ID NO: 1081.

In some embodiments, the antisense strand comprises SEQ ID NO: 1077 and its corresponding sense strand comprises SEQ ID NO: 1082. In some embodiments, the antisense strand consists of SEQ ID NO: 1077 and its corresponding sense strand consists of SEQ ID NO: 1082. In some embodiments, the antisense strand essentially consists of SEQ ID NO: 1077 and its corresponding sense strand essentially consists of SEQ ID NO: 1082.

In some embodiments, the antisense strand comprises SEQ ID NO: 1078 and its corresponding sense strand comprises SEQ ID NO: 1083. In some embodiments, the antisense strand consists of SEQ ID NO: 1078 and its corresponding sense strand consists of SEQ ID NO: 1083. In some embodiments, the antisense strand essentially consists of SEQ ID NO: 1078 and its corresponding sense strand essentially consists of SEQ ID NO: 1083.

In some embodiments, the antisense strand comprises SEQ ID NO: 1077 and its corresponding sense strand comprises SEQ ID NO: 1084. In some embodiments, the antisense strand consists of SEQ ID NO: 1077 and its corresponding sense strand consists of SEQ ID NO: 1084. In some embodiments, the antisense strand essentially consists of SEQ ID NO: 1077 and its corresponding sense strand essentially consists of SEQ ID NO: 1084.

In some embodiments of the isolated oligonucleotide of the present disclosure, the isolated oligonucleotide attenuates expression of the ACVR1C mRNA by at least 50% (e.g., 50% to 55%, 55% to 60%, 60% to 65%, 65% to 70%, 70% to 75%, 75% to 80%, 80% to 85%, 85% to 90%, 90% to 95% or 95% to 100%) at a dose of 0.4 nM as shown in Table 7.

In some embodiments of the isolated oligonucleotide of the present disclosure, the isolated oligonucleotide attenuates expression of the ACVR1C mRNA by 20% to 50% (e.g., 20% to 25%, 25% to 30%, 30% to 35%, 35% to 40%, 40% to 45% or 45% to 50%) as shown in Table 7.

In some embodiments of the isolated oligonucleotide of the present disclosure, the isolated oligonucleotide attenuates expression of the ACVR1C mRNA by at least 50% (e.g., 50% to 55%, 55% to 60%, 60% to 65%, 65% to 70%, 70% to 75%, 75% to 80%, 80% to 85%, 85% to 90%, 90% to 95% or 95% to 100%) at a dose of 0.08 nM as shown in Table 7.

In some embodiments of the isolated oligonucleotide of the present disclosure, the isolated oligonucleotide attenuates expression of the ACVR1C mRNA by 20% to 50% (e.g., 20% to 25%, 25% to 30%, 30% to 35%, 35% to 40%, 40% to 45% or 45% to 50%) as shown in Table 7.

In some embodiments of the isolated oligonucleotide of the present disclosure, the isolated oligonucleotide attenuates expression of the ACVR1C mRNA in vivo by at least 50% (e.g., 50% to 55%, 55% to 60%, 60% to 65%, 65% to 70%, 70% to 75%, 75% to 80%, 80% to 85%, 85% to 90%, 90% to 95%, or 95% to 100%) at a dose of 1.0 mg/kg, as shown in Table 7.

In some embodiments of the isolated oligonucleotide of the present disclosure, the isolated oligonucleotide attenuates expression of the ACVR1C mRNA in vivo by at least 40% (e.g., 40% to 45%, 45% to 50%, 50% to 55%, 55% to 60%, 60% to 65%, 65% to 70%, 70% to 75%, 75% to 80%, 80% to 85%85% to 90%, 90% to 95%, or 95% to 100%) at a dose of 0.5 mg/kg as shown in Table 7.

In some embodiments of the isolated oligonucleotide of the present disclosure, the isolated oligonucleotide attenuates expression of the ACVR1C mRNA in vivo by at least 55% (e.g., 55% to 60%, 60% to 65%, 65% to 70%, 70% to 75%, 75% to 80%, 80% to 85%, 85% to 90%, 90% to 95%, or 95% to 100%) at a dose of 2.0 mg/kg as shown in Table 7.

In some embodiments of the isolated oligonucleotide of the present disclosure, the isolated oligonucleotide attenuates expression of the ACVR1C mRNA in vivo by at least 8% (e.g., 5% to 10%, 10% to 15%, 15% to 20%, 20% to 25%, 5^{%} to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%85% to 90%, 90% to 95%, or 95% to 100%) at a dose of 0.056 mg/kg as shown in Table 7.

In some embodiments of the isolated oligonucleotide of the present disclosure, the isolated oligonucleotide attenuates expression of the ACVR1C mRNA in vivo by at least 10% (e.g., 10% to 15%, 15% to 20%, 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, 80% to 85%85% to 90%, 90% to 95%, or 95% to 100%) at a dose of 0.167 mg/kg as shown in Table 7.

In some embodiments, the isolated oligonucleotide of the present disclosure can comprise a linker, sometimes referred to as a loop. siRNAs comprising a linker or loop are sometimes referred to as short hairpin RNAs (shRNAs). In some embodiments, both the sense and the antisense regions of the siRNA are encoded by one single-stranded RNA. In these embodiments, and the antisense region and the sense region hybridize to form a duplex region. The sense and antisense regions are joined by a linker sequence, forming a “hairpin” or “stem-loop” structure. The siRNA can have complementary sense and antisense regions at opposing ends of a single stranded molecule, so that the molecule can form a duplex region with the complementary sequence portions, and the strands are linked at one end of the duplex region by a linker. The linker can be either a nucleotide or non-nucleotide linker or a combination thereof. The linker can interact with the first, and optionally, second strands through covalent bonds or non-covalent interactions.

Any suitable nucleotide linker sequence is envisaged as within the scope of the disclosure. An siRNA of this disclosure may include a nucleotide, non-nucleotide, or mixed nucleotide/non-nucleotide linker that joins the sense region of the nucleic acid to the antisense region of the nucleic acid. A nucleotide linker can be a linker of >2 nucleotides in length, for example about 3, 4, 5, 6, 7, 8, 9, 10, 11, 12, 13, 14, 15 or 16 nucleotides in length.

Examples of a non-nucleotide linker include an abasic nucleotide, polyether, polyamine, polyamide, peptide, carbohydrate, lipid, polyhydrocarbon, or other polymeric agents, for example polyethylene glycols such as those having from 2 to 100 ethylene glycol units. Some examples are well known in the art.

Examples of nucleotide linker sequences include, but are not limited to, AUG, CCC, UUCG, CCACC, AAGCAA, CCACACC and UUCAAGAGA.

In some embodiments, the isolated oligonucleotide of the present disclosure is an siRNA that can be a dsRNA of a length suitable as a Dicer substrate, which can be processed to produce a RISC active siRNA molecule. See, e.g., Rossi et al., US2005/0244858.

A Dicer substrate double stranded RNA (dsRNA) can be of a length sufficient that it is processed by Dicer to produce an active siRNA, and may further include one or more of the following properties: (i) the Dicer substrate dsRNA can be asymmetric, for example, having a 3′ overhang on the antisense strand, (ii) the Dicer substrate dsRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can from 19-30 bp in length.

In some embodiments, the isolated oligonucleotide of the present disclosure comprises at least one modified nucleotide. In some embodiments of the isolated oligonucleotide of the present disclosure, the sense strand or the antisense strand or both comprise one or more modified nucleotide(s). In some embodiments, only the sense strand comprises one or more modified nucleotide(s). In some embodiments, only the antisense strand comprises one or more modified nucleotide(s). In some embodiments, both the sense strand and antisense strand comprise one or more modified nucleotide(s). In some embodiments, the isolated oligonucleotide is partially chemically modified. In some embodiments, the isolated oligonucleotide is fully chemically modified.

In some embodiments, the isolated oligonucleotide comprises at least two modified nucleotides. In some embodiments, the isolated oligonucleotide comprises at least three modified nucleotides. In some embodiments, the isolated oligonucleotide comprises at least four modified nucleotides. In some embodiments, the isolated oligonucleotide comprises at least five modified nucleotides. In some embodiments, the isolated oligonucleotide comprises at least six modified nucleotides. In some embodiments, the isolated oligonucleotide comprises at least seven modified nucleotides. In some embodiments, the isolated oligonucleotide comprises at least eight modified nucleotides. In some embodiments, the isolated oligonucleotide comprises at least nine modified nucleotides. In some embodiments, the isolated oligonucleotide comprises at least ten modified nucleotides. In some embodiments, the isolated oligonucleotide comprises at least eleven modified nucleotides. In some embodiments, the isolated oligonucleotide comprises at least twelve modified nucleotides. In some embodiments, the isolated oligonucleotide comprises at least thirteen modified nucleotides. In some embodiments, the isolated oligonucleotide comprises at least fourteen modified nucleotides. In some embodiments, the isolated oligonucleotide comprises at least fifteen modified nucleotides. In some embodiments, the isolated oligonucleotide comprises at least sixteen modified nucleotides. In some embodiments, the isolated oligonucleotide comprises at least seventeen modified nucleotides. In some embodiments, the isolated oligonucleotide comprises at least eighteen modified nucleotides. In some embodiments, the isolated oligonucleotide comprises at least nineteen modified nucleotides. In some embodiments, the isolated oligonucleotide comprises at least twenty modified nucleotides. In some embodiments, the isolated oligonucleotide comprises more than twenty modified nucleotides. In some embodiments, the isolated oligonucleotide comprises between twenty and thirty modified nucleotides. In some embodiments, the isolated oligonucleotide comprises between thirty and forty modified nucleotides. In some embodiments, the isolated oligonucleotide comprises between forty and fifty modified nucleotides.

In some embodiments, the sense strand and/or the antisense strand of the isolated oligonucleotide each comprise at least one modified nucleotides. In some embodiments, the sense strand and/or the antisense strand of the isolated oligonucleotide each comprise at least two modified nucleotides. In some embodiments, the sense strand and/or the antisense strand of the isolated oligonucleotide each comprise at least three modified nucleotides. In some embodiments, the sense strand and/or the antisense strand of the isolated oligonucleotide each comprise at least four modified nucleotides. In some embodiments, the sense strand and/or the antisense strand of the isolated oligonucleotide each comprise at least five modified nucleotides. In some embodiments, the sense strand and/or the antisense strand of the isolated oligonucleotide each comprise at least six modified nucleotides. In some embodiments, the sense strand and/or the antisense strand of the isolated oligonucleotide each comprise at least seven modified nucleotides. In some embodiments, the sense strand and/or the antisense strand of the isolated oligonucleotide each comprise at least eight modified nucleotides. In some embodiments, the sense strand and/or the antisense strand of the isolated oligonucleotide each comprise at least nine modified nucleotides. In some embodiments, the sense strand and/or the antisense strand of the isolated oligonucleotide each comprise at least ten modified nucleotides. In some embodiments, the sense strand and/or the antisense strand of the isolated oligonucleotide each comprise at least eleven modified nucleotides. In some embodiments, the sense strand and/or the antisense strand of the isolated oligonucleotide each comprise at least twelve modified nucleotides. In some embodiments, the sense strand and/or the antisense strand of the isolated oligonucleotide each comprise at least thirteen modified nucleotides. In some embodiments, the sense strand and/or the antisense strand of the isolated oligonucleotide each comprise at least fourteen modified nucleotides. In some embodiments, the sense strand and/or the antisense strand of the isolated oligonucleotide each comprise at least fifteen modified nucleotides. In some embodiments, the sense strand and/or the antisense strand of the isolated oligonucleotide each comprise at least sixteen modified nucleotides. In some embodiments, the sense strand and/or the antisense strand of the isolated oligonucleotide each comprise at least seventeen modified nucleotides. In some embodiments, the sense strand and/or the antisense strand of the isolated oligonucleotide each comprise at least eighteen modified nucleotides. In some embodiments, the sense strand and/or the antisense strand of the isolated oligonucleotide each comprise at least nineteen modified nucleotides. In some embodiments, the sense strand and/or the antisense strand of the isolated oligonucleotide each comprise at least twenty modified nucleotides.

In some embodiments, wherein the isolated oligonucleotide comprises more than one modified nucleotide, at least a first nucleotide comprises a first modification and at least a second nucleotide comprises a second modification. In some embodiments, the first modification and second modification are different. In some embodiments, the at least first nucleotide and the at least second nucleotide are located on different strands of the isolated oligonucleotide. In some embodiments, the at least first nucleotide and the at least second nucleotide are located on the same strand of the isolated oligonucleotide.

In some embodiments of the isolated oligonucleotide, wherein the isolated oligonucleotide comprises more than one modified nucleotide, at least a first modified nucleotide comprises a first modification, and at least a second modified nucleotide comprises a second modification, and at least a third nucleotide comprises a third modification. In some embodiments, the isolated oligonucleotide comprises a first, a second, a third and a fourth modifications. In some embodiments, the isolated oligonucleotide comprises more than four modifications. In some embodiments, all modifications are on the sense strand. In some embodiments, all modifications are on the antisense strand. Any combination of locations of the modifications between the sense strand and antisense strand is envisaged within the isolated oligonucleotides of the present disclosure.

In some embodiments, the modified nucleotides are consecutively located on the sense strand or the antisense strand or both. In some embodiments, some but not all of the modified nucleotides are consecutively located on the sense strand or the antisense strand or both. In some embodiments, the modified nucleotides on the sense strand or the antisense strand or both are not consecutively located.

Envisaged within the present disclosure is an isolated oligonucleotide, wherein any nucleotide on the sense strand or antisense strand can be modified. In some embodiments, any nucleotide on the antisense strand can be modified. In some embodiments, any nucleotide on the antisense strand can be modified.

In some embodiments of the isolated oligonucleotides of the present disclosure, the sense strand or the antisense strand or both comprise one or more modified nucleotide(s). In some embodiments, only the sense strand comprises one modified nucleotide. In some embodiments, only the sense strand comprises one or more modified nucleotide(s). In some embodiments, only the antisense strand comprises one modified nucleotide. In some embodiments, only the antisense strand comprises one or more modified nucleotide(s).

In some embodiments, the isolated oligonucleotides of the present disclosure comprises at least one modified nucleotide(s). In some embodiments, the one or more modified nucleotide(s) increases the stability or potency or both of the isolated oligonucleotide. In some embodiments, the one or more modified nucleotide(s) increases the stability of the RNA duplex, and siRNA.

Modifications that increase RNA stability include, but are not limited to, locked nucleic acids. As used herein, the term “locked nucleic acid” or “LNA” includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. LNAs having a 2′-4′ cyclic linkage, as described in the International Patent Application WO 99/14226, WO 00/56746, WO 00/56748, and WO 00/66604, the contents of each of which are incorporated herein by reference.

In some embodiments of the isolated oligonucleotide of the present disclosure, the sense strand or the antisense strand or both comprise at least one nucleotide having a modified phosphate backbone. In some embodiments, the sense strand of the isolated oligonucleotide comprises at least one nucleotide having a modified phosphate backbone. In some embodiments, the antisense strand of the isolated oligonucleotide comprises at least one nucleotide having a modified phosphate backbone. In some embodiments, wherein the isolated oligonucleotide of the present disclosure comprises a modified phosphate backbone, the modified phosphate backbone comprises a modified phosphodiester bond. In some embodiments, the modified phosphodiester bond is modified by replacing one or more oxygen atoms with a moiety, wherein the moiety is bonded to the phosphorus atom in the phosphodiester bond with a carbon, nitrogen, or sulfur atom in the moiety, or by forming a 2′-5′ linkage. In some embodiments, the modified phosphodiester bond comprises phosphorothioate, phosphorodithioate, methylphosphonate, phosphoramidate diester, mesyl phosphoramidate, or phosphonoacetate.

In some embodiments, the isolated oligonucleotide of the present disclosure comprises one or more non-natural base-containing nucleotide, a locked nucleotide, or an abasic nucleotide. In some embodiments, the one or more modified nucleotide comprises a phosphorothioate derivative or an acridinine substituted nucleotide. In some embodiments, the isolated oligonucleotides of the present disclosure comprise a phosphate mimic at the 5′-terminus of antisense strand, including but not limited to vinylphosphonate or other phosphate analogues. In some embodiments, the 5′-phosphate mimic is ethylphosphonate, vinylphosphonate or an analog thereof.

In some embodiments, the modified nucleotide comprises 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl-aminomethyluracil, 5-methoxyaminomethyl-2-thiouracil, beta-D-mannosylqueosine, 5′-methoxycarboxymethyluracil, 5-methoxyuracil, 2-methylthio-N-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2, 6-diaminopurine.

In some embodiments of the isolated oligonucleotide of the present disclosure, the sense strand or the antisense strand or both comprise a terminal or internal nucleotide linked to one or more targeting ligands. In some embodiments, the terminal or internal nucleotide is linked to the one or more targeting ligands directly. In some embodiments, the terminal or internal nucleotide is linked to the one or more targeting ligands indirectly by a linker. In some embodiments, the one or more targeting ligands linked directly or indirectly to the terminal or internal nucleotide can further comprise a PK modulator. In some embodiments, the PK modulator is a competitive modulator, a positive allosteric modulator, a negative allosteric modulator or a neutral allosteric modulator. In some embodiments, the targeting ligand is selected from one or more of a carbohydrate, a peptide, a lipid, an antibody or a fragment thereof, an aptamer, an albumin, a fibrinogen, and a folate.

### Modification of the Nucleotides

Provided herein is an isolated oligonucleotide, comprising: (a) a sense strand comprising X1 nucleotides, wherein at least one nucleotide is modified with a first modification, each of the remaining nucleotides is independently modified with a second modification, and X1 is an integer selected from 13-36, wherein the first modification and the second modification are different; and (b) an antisense strand comprising X2 nucleotides, wherein at least one nucleotide is modified with a third modification, each of the remaining nucleotides is independently modified with a fourth modification, and X2 is an integer selected from 18-31, wherein the third modification and the fourth modification are different.

In some embodiments, the X1 nucleotides of the sense strand of the isolated oligonucleotide of the present disclosure is 18-21 and the X2 nucleotides of the antisense strand of the isolated oligonucleotide of the present disclosure is 20-23. In some embodiments, the X1 nucleotides of the sense strand of the isolated oligonucleotide of the present disclosure is 20 or 21 and the X2 nucleotides of the antisense strand of the isolated oligonucleotide of the present disclosure is 22 or 23. In some embodiments, the X2 nucleotides of the antisense strand of the isolated oligonucleotide of the present disclosure equals the X1 nucleotides of the sense strand of the isolated oligonucleotide of the present disclosure plus 2. In some embodiments, the X1 nucleotides of the sense strand of the isolated oligonucleotide of the present disclosure is 21 and the X2 nucleotides of the antisense strand of the isolated oligonucleotide of the present disclosure is 23. In some embodiments, the X1 nucleotides of the sense strand of the isolated oligonucleotide of the present disclosure is 20 and the X2 nucleotides of the antisense strand of the isolated oligonucleotide of the present disclosure is 22.

In some embodiments of the isolated oligonucleotide of the present disclosure, the isolated oligonucleotide comprises: (a) a sense strand comprising 20 nucleotides, wherein at least one nucleotide is modified with a first modification, each of the remaining nucleotides is independently modified with a second modification, wherein the first modification and the second modification are the same or different; and (b) an antisense strand comprising 22 nucleotides, wherein at least one nucleotide is modified with a third modification, each of the remaining nucleotides is independently modified with a fourth modification, wherein the third modification and the fourth modification are the same or different.

In some embodiments, the first modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the first modification is 2′-F modification, 2′-CN modification, 2′-N3 modification, or 2′-deoxy modification, or a stereoisomer thereof. In some embodiments, the first modification is 2′-F modification, 2′-CN modification, or 2′-N3 modification, or a stereoisomer thereof. In some embodiments, the first modification is 2′-F modification or a stereoisomer thereof.

In some embodiments, the second modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the second modification is 2′-OR modification, or morpholino replacement, or a combination thereof. In some embodiments, the second modification is 2′-OR modification. In some embodiments, the second modification is 2′-O-methyl modification or 2′-methoxyethoxy modification. In some embodiments, the second modification is 2′-O-methyl modification. In some embodiments, the second modification is morpholino replacement.

In some embodiments, the first modification is 2′-F modification or a stereoisomer thereof, and the second modification is 2′-O-methyl modification or 2′-methoxyethoxy modification. In some embodiments, the first modification is 2′-F modification or a stereoisomer thereof, and the second modification is 2′-O-methyl modification.

In some embodiments, the third modification is modification of the sugar moiety of the at least one nucleotide at the 2′-position selected from 2′-F modification, 2′-CN modification, 2′-N3 modification, 2′-deoxy modification, and an equivalent thereof, and a combination thereof. In some embodiments, the third modification is 2′-F modification, 2′-CN modification, 2′-N3 modification, or 2′-deoxy modification, or a stereoisomer thereof. In some embodiments, the third modification is 2′-F modification, 2′-CN modification, or 2′-N₃ modification, or a stereoisomer thereof. In some embodiments, the third modification is 2′-F modification or a stereoisomer thereof.

In some embodiments, the fourth modification is modification of the sugar moiety of one or more of the remaining nucleotides at the 2′-position selected from 2′-C₁-C₆ alkyl, 2′-OR modification wherein R is C₁-C₆ alkyl optionally substituted with C₁-C₆ alkoxy, acetamide, phenyl, or heteroaryl comprising a 5- or 6-membered ring and 1 or 2 heteroatoms selected from N, O, and S, 2′-amino, and morpholino replacement, and an equivalent thereof, and a combination thereof. In some embodiments, the fourth modification is 2′-OR modification, or morpholino replacement, or a combination thereof. In some embodiments, the fourth modification is 2′-OR modification. In some embodiments, the fourth modification is 2′-O-methyl modification or 2′-methoxyethoxy modification. In some embodiments, the fourth modification is 2′-O-methyl modification. In some embodiments, the fourth modification is morpholino replacement.

In some embodiments, the third modification is 2′-F modification or a stereoisomer thereof, and the fourth modification is 2′-O-methyl modification or 2′-methoxyethoxy modification. In some embodiments, the third modification is 2′-F modification or a stereoisomer thereof, and the fourth modification is 2′-O-methyl modification.

### Sense Strand

In some embodiments of the isolated oligonucleotide of the present disclosure comprising a sense and an antisense strand, in the sense strand of the isolated oligonucleotide of the present disclosure, at least three nucleotides are modified with the first modification. In some embodiments, in the sense strand of the isolated oligonucleotide of the present disclosure, at least two of the at least three nucleotides modified with the first modification are consecutively located. In some embodiments, in the sense strand of the isolated oligonucleotide of the present disclosure, at least three of the at least three nucleotides modified with the first modification are consecutively located.

In some embodiments, in the sense strand of the isolated oligonucleotide of the present disclosure, in the sense strand at least four nucleotides are modified with the first modification. In some embodiments, in the sense strand of the isolated oligonucleotide of the present disclosure, at least three of the at least four nucleotides modified with the first modification are consecutively located. In some embodiments, in the sense strand of the isolated oligonucleotide of the present disclosure, at least four of the at least four nucleotides modified with the first modification are consecutively located.

In some embodiments, in the sense strand of the isolated oligonucleotide of the present disclosure, in the sense strand at least five nucleotides are modified with the first modification. In some embodiments, in the sense strand of the isolated oligonucleotide of the present disclosure, at least three of the at least five nucleotides modified with the first modification are consecutively located. In some embodiments, in the sense strand of the isolated oligonucleotide of the present disclosure, at least four of the at least five nucleotides modified with the first modification are consecutively located.

In some embodiments, in the sense strand of the isolated oligonucleotide of the present disclosure, the at least three nucleotides, the at least four nucleotides, or the at least five nucleotides modified with the first modification are located from position 10 to position 15 from the nucleotide complementary to the first nucleotide at the 5′-terminus of the antisense strand.

In some embodiments, in the sense strand of the isolated oligonucleotide of the present disclosure, two of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions selected from position 10, 11, 12, and 13 from the nucleotide complementary to the first nucleotide at the 5′-terminus of the antisense strand. In some embodiments, in the sense strand of the isolated oligonucleotide of the present disclosure, one of the at least three nucleotides modified with the first modification is located at position 11 from the nucleotide complementary to the first nucleotide at the 5′-terminus of the antisense strand.

In some embodiments, in the sense strand of the isolated oligonucleotide of the present disclosure, three of the at least three nucleotides modified with the first modification are located at positions 11, 12 and 13 from the nucleotide complementary to the first nucleotide at the 5′-terminus of the antisense strand. In some embodiments, in the sense strand of the isolated oligonucleotide of the present disclosure, three of the at least three nucleotides modified with the first modification are located at positions 12, 13 and 14 from the nucleotide complementary to the first nucleotide at the 5′-terminus of the antisense. In some embodiments, in the sense strand of the isolated oligonucleotide of the present disclosure, three of the at least three nucleotides modified with the first modification are located at positions 10, 11 and 12 from the nucleotide complementary to the first nucleotide at the 5′-terminus of the antisense strand.

In some embodiments, in the sense strand of the isolated oligonucleotide of the present disclosure, one of the at least four nucleotides modified with the first modification is located at position 10 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 11 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 12 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 13 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 14 from the nucleotide complementary to the first nucleotide at the 5′-terminus of the antisense strand. In some embodiments, in the sense strand of the isolated oligonucleotide of the present disclosure, one of the at least four nucleotides modified with the first modification is located at position 15 from the nucleotide complementary to the first nucleotide at the 5′-terminus of the antisense strand.

In some embodiments, in the sense strand of the isolated oligonucleotide of the present disclosure, the at least four nucleotides modified with the first modification are located at positions 10, 11, 12 and 13 from the nucleotide complementary to the first nucleotide at the 5′-terminus of the antisense strand.

In some embodiments, in the sense strand of the isolated oligonucleotide of the present disclosure, the at least five nucleotides modified with the first modification are located at positions 10, 11, 12, 13 and 15 from the nucleotide complementary to the first nucleotide at the 5′-terminus of the antisense strand.

In some embodiments of the isolated oligonucleotide of the present disclosure, the sense strand comprises five nucleotides modified with the first modification, wherein the five nucleotides modified with the first modification are located at positions 10, 11, 12, 13 and 15 from the nucleotide complementary to the first nucleotide at the 5′-terminus of the antisense strand.

In some embodiments, in the sense strand of the isolated oligonucleotide of the present disclosure, not all of the at least three nucleotides, the at least four nucleotides, or the at least five nucleotides modified with the first modification are consecutively located. In some embodiments, in the sense strand of the isolated oligonucleotide of the present disclosure, the at least three nucleotides, the at least four nucleotides, or the at least five nucleotides are modified with 2′-F modification.

In some embodiments, the sense strand of the isolated oligonucleotide of the present disclosure comprises nucleotides modified with 2′-F modification (“F”), and nucleotides modified with 2′-O-methyl modification (“M”), according to the formula: 5′ (M)_{g}(F)_{f}(M)ₑ(F)_{d}(M)_{c}(F)_{b}(M)ₐ 3′, wherein M is 2′-O-methyl modified nucleotide, F is 2′-F modified nucleotide, and a, b, c, d, e, f and g are each independently any one of 0-16, and wherein the sense strand is 5′(M)₀(F)₀(M)₅(F)₁(M)₁(F)₄(M)₉ 3′.

### Antisense Strand

In some embodiments, in the antisense strand of the isolated oligonucleotide of the present disclosure, at most seven nucleotides are modified with the third modification.

In some embodiments, in the antisense strand of the isolated oligonucleotide of the present disclosure, at most four of the at most seven nucleotides modified with the third modification are located from position 2 to position 8 from the first nucleotide at the 5′-terminus of the antisense strand. In some embodiments, in the antisense strand of the isolated oligonucleotide of the present disclosure, at least one of the at most seven nucleotides are modified with the third modification is located at position 2 from the first nucleotide at the 5′-terminus of the antisense strand.

In some embodiments, in the antisense strand of the isolated oligonucleotide of the present disclosure, at most two of the at most seven nucleotides modified with the third modification are consecutively located. In some embodiments, in the antisense strand of the isolated oligonucleotide of the present disclosure, the at most two consecutively located of the at most seven nucleotides modified with the third modification are located at positions 2 and 3 from the first nucleotide at the 5′-terminus of the antisense strand.

In some embodiments, in the antisense strand of the isolated oligonucleotide of the present disclosure, at least one of the at most seven nucleotides modified with the third modification is located at position 14 from the first nucleotide at the 5′-terminus of the antisense strand. In some embodiments, in the antisense strand of the isolated oligonucleotide of the present disclosure, two or three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, three of the at most seven nucleotides modified with the third modification are located at positions selected from position 2, 3, 5, and 6 from the first nucleotide at the 5′-terminus of the antisense strand. In some embodiments, in the antisense strand of the isolated oligonucleotide of the present disclosure, two of the at most seven nucleotides modified with the third modification are located at positions 2 and 5 from the first nucleotide at the 5′-terminus of the antisense strand. In some embodiments, in the antisense strand of the isolated oligonucleotide of the present disclosure, two of the at most seven nucleotides modified with the third modification are located at positions 2 and 3 from the first nucleotide at the 5′-terminus of the antisense strand.

In some embodiments, in the antisense strand of the isolated oligonucleotide of the present disclosure, three of the at most seven nucleotides modified with the third modification are located at positions 2, 3 and 5 from the first nucleotide at the 5′-terminus of the antisense strand.

In some embodiments, in the antisense strand of the isolated oligonucleotide of the present disclosure, one or two of the at most seven nucleotides modified with the third modification are located at positions selected from position 14 and 16 from the first nucleotide at the 5′-terminus of the antisense strand. In some embodiments, in the antisense strand of the isolated oligonucleotide of the present disclosure, two of the at most seven nucleotides modified with the third modification are located at positions 14 and 16 from the first nucleotide at the 5′-terminus of the antisense strand. In some embodiments, in the antisense strand of the isolated oligonucleotide of the present disclosure, the at most seven nucleotides are modified with 2′-F modification. In some embodiments, in the antisense strand of the isolated oligonucleotide of the present disclosure, one of the at most seven nucleotides modified with the third modification is located at position 14 from the first nucleotide at the 5′-terminus of the antisense strand. In some embodiments, in the antisense strand of the isolated oligonucleotide of the present disclosure, two of the at most seven nucleotides modified with the third modification is located at positions 14 and 16 from the first nucleotide at the 5′-terminus of the antisense strand.

In some embodiments of the isolated oligonucleotides of the present disclosure, wherein the antisense strand comprises at most seven nucleotides modified with the third modification, the at most seven nucleotides are modified with 2′-F modification. In some embodiments, in the antisense strand of the isolated oligonucleotide of the present disclosure, one of the at most seven nucleotides modified with the third modification is located at position 2 from the first nucleotide at the 5′-terminus of the antisense strand. In some embodiments, in the antisense strand of the isolated oligonucleotide of the present disclosure, one of the at most seven nucleotides modified with the third modification is located at position 3 from the first nucleotide at the 5′-terminus of the antisense strand. In some embodiments, in the antisense strand of the isolated oligonucleotide of the present disclosure, one of the at most seven nucleotides modified with the third modification is located at position 5 from the first nucleotide at the 5′-terminus of the antisense strand. In some embodiments, in the antisense strand of the isolated oligonucleotide of the present disclosure, one of the at most seven nucleotides modified with the third modification is located at position 7 from the first nucleotide at the 5′-terminus of the antisense strand. In some embodiments, in the antisense strand of the isolated oligonucleotide of the present disclosure, one of the at most seven nucleotides modified with the third modification is located at position 10 from the first nucleotide at the 5′-terminus of the antisense strand. In some embodiments, in the antisense strand of the isolated oligonucleotide of the present disclosure, one of the at most seven nucleotides modified with the third modification is located at position 14 from the first nucleotide at the 5′-terminus of the antisense strand. In some embodiments, in the antisense strand of the isolated oligonucleotide of the present disclosure, one of the at most seven nucleotides modified with the third modification is located at position 16 from the first nucleotide at the 5′-terminus of the antisense strand. In some embodiments, in the antisense strand of the isolated oligonucleotide of the present disclosure, the at most seven nucleotides modified with the third modification are located at positions 2, 3, 5, 7, 10, 14 and 16 from the first nucleotide at the 5′-terminus of the antisense strand.

In some embodiments, in the antisense strand of the isolated oligonucleotide of the present disclosure, the antisense strand comprises nucleotides modified with 2′-F modification (“F”), and nucleotides modified with 2′-O-methyl modification (“M”), according to the formula: 3′ (M)ₐ(F)_{b}(M)_{c}(F)_{d}(M)ₑ(F)_{f}(M)_{g}(F)ₕ(M)ᵢ(F)ⱼ(M)ₖ(F)ₗ(M)ₘ(F)ₙ(M)ₒ 5′, wherein M is 2′-O-methyl modified nucleotide, F is 2′-F modified nucleotide, and a, b, c, d, e, f, g, h, i, j, k, 1, m, n and o are each independently any one of 0-16, wherein the antisense strand is any one of: 3′(M)₀(F)₀(M)₆(F)₁(M)₁(F)₁(M)₃(F)₁(M)₂(F)₁(M)₁(F)₁(M)₁(F)₂(M)₁ 5′.

### Targeting Ligand

In some embodiments, in the sense strand or the antisense strand or both of the isolated oligonucleotides of the present disclosure, a terminal or internal nucleotide is linked to a targeting ligand. In some embodiments, the targeting ligand is attached to one or more nucleotides at the 5′ end of the sense strand of the isolated oligonucleotide of the present disclosure. In some embodiments, the targeting ligand is attached to one or more nucleotides at the 3′ end of the sense strand of the isolated oligonucleotide of the present disclosure. In some embodiments, the targeting ligand is attached to one or more nucleotides at the 5′ end of the antisense strand of the isolated oligonucleotide of the present disclosure. In some embodiments, the targeting ligand is attached to one or more nucleotides at the 3′ end of the antisense strand of the isolated oligonucleotide of the present disclosure. In some embodiments, the targeting ligand is attached to one or more nucleotides of the at least two single-stranded nucleotides at the 3′-terminus of the antisense strand of the isolated oligonucleotide of the present disclosure.

In some embodiments, the targeting ligand is selected from one or more of a carbohydrate, a peptide, a lipid, an antibody or a fragment thereof, an aptamer, an albumin, a fibrinogen, and a folate. In some embodiments, the targeting ligand binds to a surface protein on a cell expressing a target mRNA of the isolated oligonucleotide of the present disclosure. In some embodiments, the targeting ligand mediates entry of the isolated oligonucleotide of the present disclosure, into a cell expressing a target mRNA of the isolated oligonucleotide of the present disclosure.

In some embodiments, the targeting ligand is an adipose-targeting ligand.

In some embodiments, the targeting ligand is a therapeutic ligand. In some embodiments, the targeting ligand is a therapeutic antibody.

In some embodiments, the targeting ligand is attached to the isolated oligonucleotide of the present disclosure by a linker. In some embodiments, the linker is any one or a protein, a DNA, an RNA or a chemical compound. In some embodiments, the isolated oligonucleotide, the linker and the targeting ligand, of the present disclosure form a scaffold. As used herein, the term “scaffold” refers to a compound or complex that comprises a linker of the present disclosure, wherein the linker is covalently attached to either a ligand or an isolated oligonucleotide or both.

In some embodiments, the isolated oligonucleotide, the linker and the targeting ligand, of the present disclosure form a conjugate. As used herein, the term “conjugate” refers to a compound or complex that comprises an isolated oligonucleotide being covalently attached to a ligand via a linker of the present disclosure.

As used herein, the term “targeting ligand” or “ligand” refers to a moiety that is capable of mediating its entry into, or facilitating or allowing its delivery to, a target site (e.g., a target cell or tissue). In some embodiments, the targeting ligand comprises a lipid ligand moiety, which may direct the systemic, central nervous system, peripheral nervous system, and/or ocular delivery of an oligonucleotide. In some embodiments, the targeting ligand comprises a saccharide ligand moiety, which may direct the enhance the delivery and cellular uptake of an oligonucleotide (e.g., in macrophage cells in liver and other tissues).

In some embodiments, the targeting ligand binds to the asialoglycoprotein receptor (ASGPR). In some embodiments, the targeting ligand binds to (e.g., through ASGPR) the liver, such as the parenchymal cells of the liver.

Suitable targeting ligands include, but are not limited to, the ligands disclosed in Winkler (Ther. Deliv., 2013, 4(7): 791-809), PCT Patent Appl'n Pub. Nos. WO/2016/100401, WO/2012/089352, and WO/2009/082607, and U.S. Patent Appl'n Pub. Nos. 2009/0239814, 2012/0136042, 2013/0158824, and 2009/0247608, each of which is incorporated by reference.

In some embodiments, the targeting ligand comprises a carbohydrate moiety.

As used herein, “carbohydrate moiety” refers to a moiety which comprises one or more monosaccharide units each having at least six carbon atoms (which may be linear, branched or cyclic), with an oxygen, nitrogen or sulfur atom bonded to each carbon atom. In some embodiments, the carbohydrate moiety comprises a monosaccharide, a disaccharide, a trisaccharide, or a tetrasaccharide. In some embodiments, the carbohydrate moiety comprises an oligosaccharide containing from about 4-9 monosaccharide units. In some embodiments, the carbohydrate moiety comprises a polysaccharide (e.g., a starch, a glycogen, a cellulose, or a polysaccharide gum).

In some embodiments, the carbohydrate moiety comprises a monosaccharide, a disaccharide, a trisaccharide, or a tetrasaccharide. In some embodiments, the carbohydrate moiety comprises an oligosaccharide (e.g., containing from about four to about nine monosaccharide units). In some embodiments, the carbohydrate moiety comprises a polysaccharide (e.g., a starch, a glycogen, a cellulose, or a polysaccharide gum).

In some embodiments, the ligand is capable of binding to a human asialoglycoprotein receptor (ASGPR), e.g., human asialoglycoprotein receptor 2 (ASGPR2).

In some embodiments, the carbohydrate moiety comprises a sugar (e.g., one, two, or three sugar). In some embodiments, the carbohydrate moiety comprises galactose or a derivative thereof (e.g., one, two, or three galactose or the derivative thereof). In some embodiments, the carbohydrate moiety comprises N-acetylgalactosamine or a derivative thereof (e.g., one, two, or three N-acetylgalactosamine or the derivative thereof). In some embodiments, the carbohydrate moiety comprises N-acetyl-D-galactosylamine or a derivative thereof (e.g., one, two, or three N-acetyl-D-galactosylamine or the derivative thereof).

In some embodiments, the carbohydrate moiety comprises N-acetylgalactosamine (e.g., one, two, or three N-acetylgalactosamine). In some embodiments, the carbohydrate moiety comprises N-acetyl-D-galactosylamine (e.g., one, two, or three N-acetyl-D-galactosylamine).

In some embodiments, the carbohydrate moiety comprises mannose or a derivative thereof (e.g., mannose-6-phosphate). In some embodiments, the carbohydrate moiety further comprises a linking moiety that connects the one or more sugar (e.g., N-acetyl-D-galactosylamine) with a linker.

In some embodiments the linker comprises thioether (e.g., thiosuccinimide, or the hydrolysis analogue thereof), disulfide, triazole, phosphorothioate, phosphodiester, ester, amide, or any combination thereof. In some embodiments, the linker is a triantennary linking moiety. Suitable targeting ligands include, but are not limited to, the ligands disclosed in PCT Appl'n Pub. Nos. WO/2015/006740, WO/2016/100401, WO/2017/214112, WO/2018/039364, and WO/2018/045317, each of which is incorporated herein by reference.

In some embodiments, the targeting ligand comprises a lipid or a lipid moiety (e.g., one, two, or three lipid moiety). In some embodiments the lipid moiety comprises (e.g., one, two, of three of) C₈-C₂₄ fatty acid, cholesterol, vitamin, sterol, phospholipid, or any combination thereof.

In some embodiments, the targeting ligand comprises:

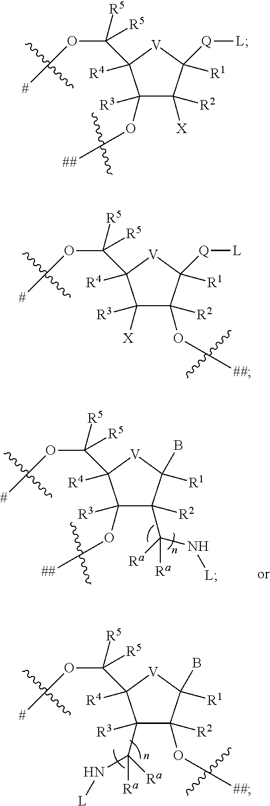

wherein:
   L is a lipid moiety;
   B is H, C₁-C₆ alkyl, or a nucleobase moiety;
   V is —O—, —NR^{V}—, or —C(R^{V})₂—;
   each R^{V} independently is H or C₁-C₆ alkyl optionally substituted with one or more halogen;
   Q is —(CR^{a}R^{a})ₙ—NH—* in which * denotes attachment to L; or Q is C₆-C₁₀ arylene or 5- to 10-membered heteroarylene, wherein the C₆-C₁₀ arylene or 5- to 10-membered heteroarylene is optionally substituted with one or more R^{Q};
   each R^{Q} independently is halogen or C₁-C₆ alkyl optionally substituted with one or more halogen;
   X is H, halogen, or —OR^{X};
   R^{X} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
   each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
   each R^{a} independently is H, halogen, or C₁-C₆ alkyl optionally substituted with one or more halogen;
   R¹ is H, halogen, or C₁-C₆ alkyl optionally substituted with one or more halogen;
   R² is H, halogen, or C₁-C₆ alkyl optionally substituted with one or more halogen;
   R³ is H, halogen, or C₁-C₆ alkyl optionally substituted with one or more halogen;
   R⁴ is H, halogen, or C₁-C₆ alkyl optionally substituted with one or more halogen; or R⁴ and R^{X} together form C₁-C₆ alkylene;
   each R⁵ independently is H, halogen, or C₁-C₆ alkyl optionally substituted with one or more halogen;
   n is an integer ranging from about 0 to about 10; and
   each of # and ## independently is an attachment to the rest of the conjugate, or
   when the targeting ligand is at the 3′-terminus of the oligonucleotide, then: # is an attachment to the rest of the conjugate; and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano; or
   when the targeting ligand is at the 5′-terminus of the oligonucleotide, then: # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

In some embodiments, the targeting ligand comprises N-33a:

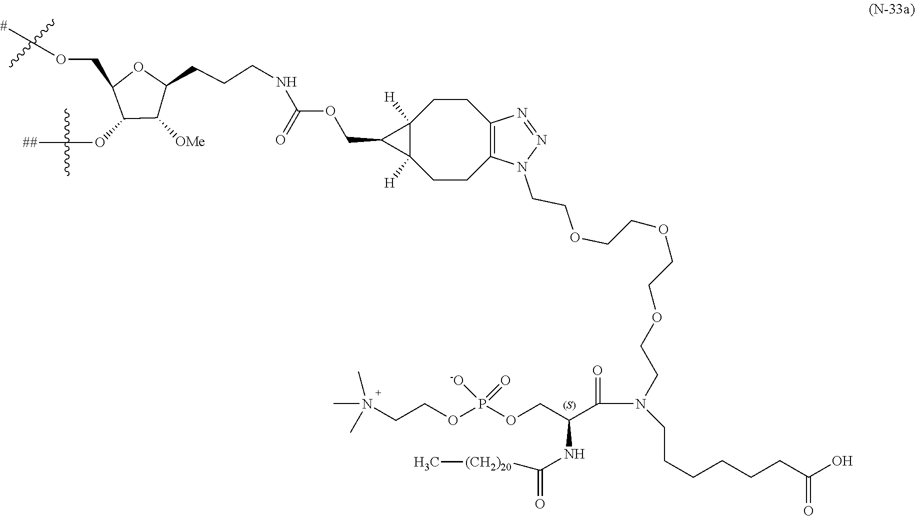

wherein:
each of # and ## independently is an attachment to the rest of the conjugate, or
when N-33a is at the 3′-terminus of the oligonucleotide, then: # is an attachment to the rest of the conjugate; and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano; or
when N-33a is at the 5′-terminus of the oligonucleotide, then: # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

In some embodiments, the targeting ligand comprises N-33a, wherein when N-33a is at the 3′-terminus of the oligonucleotide, then # is an attachment to the rest of the conjugate; and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the targeting ligand comprises N-33a, wherein when N-33a is at the 3′-terminus of the oligonucleotide, then # is an attachment to the rest of the conjugate; and ## is H.

In some embodiments, the conjugate comprises N-33a at the 3′-terminus of the oligonucleotide, wherein # is an attachment to the rest of the conjugate, and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises N-33a at the 3′-terminus of the oligonucleotide, wherein # is an attachment to the rest of the conjugate, and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises N-33a at the 3′-terminus of the oligonucleotide, wherein # is an attachment to the rest of the conjugate, and ## is H.

In some embodiments, each of # and ## independently is an attachment to the rest of the conjugate.

In some embodiments, # is an attachment to the rest of the conjugate; and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, # is an attachment to the rest of the conjugate; and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, # is an attachment to the rest of the conjugate; and ## is H.

In some embodiments, # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

In some embodiments, # is —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

In some embodiments, # is H; and ## is an attachment to the rest of the conjugate.

In some embodiments, L is a fatty acid, a glycerolipid, a glycerophospholipid, a sphingolipid, a sterol, a prenol, or a saccharolipid, any derivative thereof, or any portion thereof.

In some embodiments, L is a fatty acid, a derivative thereof, or a portion thereof.

In some embodiments, L is a glycerolipid, a derivative thereof, or a portion thereof.

In some embodiments, L is a glycerophospholipid, a derivative thereof, or a portion thereof.

In some embodiments, L is a sphingolipid, a derivative thereof, or a portion thereof.

In some embodiments, L is a sterol, a derivative thereof, or a portion thereof.

In some embodiments, L is a prenol, a derivative thereof, or a portion thereof.

In some embodiments, L is a saccharolipid, a derivative thereof, or a portion thereof.

In some embodiments, L is —C(═O)R^{L} or —C(═S)R^{L};
R^{L} is C₂-C₂₀₀ hydrocarbon chain or 2- to 200-membered hetero-hydrocarbon chain, wherein the C₂-C₂₀₀ hydrocarbon chain or 2- to 200-membered hetero-hydrocarbon chain is optionally substituted with one or more R^{L′};
each R^{L′} independently is oxo, dihalocarbene, cyano, halogen, —OH, —O(C₁-C₁₂ alkyl), —O(C₆-C₁₀ aryl), —COOH, —COO(C₁-C₁₂ alkyl), —CO(C₁-C₃₀ alkyl), —NHCO(C₁-C₃₀ alkyl), —CONH₂, —CONH(C₁-C₁₂ alkyl), —CON(C₁-C₁₂ alkyl)₂, —C(O)NHOH, —SO₃H, —SO₃(C₁-C₁₂ alkyl), —NH₂, —NH(C₁-C₁₂ alkyl), —N(C₁-C₁₂ alkyl)₂, —S(C₁-C₁₂ alkyl), —S(C₆-C₁₀ aryl), —P(C₆-C₁₀ aryl)₃, —OP(═O)O⁻)O—(C₁-C₁₂ alkylene)-NMe₃⁺, C₁-C₁₂ alkyl, C₂-C₁₂ alkenyl, C₂-C₁₂ alkynyl, C₃-C₈ cycloalkyl, 3- to 8-membered heterocycloalkyl, C₆-C₁₀ aryl, or 5- to 10-membered heteroaryl, wherein the —O(C₁-C₁₂ alkyl), —O(C₆-C₁₀ aryl), —COO(C₁-C₁₂ alkyl), —CO(C₁-C₃₀ alkyl), —NHCO(C₁-C₃₀ alkyl), —CONH(C₁-C₁₂ alkyl), —CON(C₁-C₁₂ alkyl)₂, —SO₃(C₁-C₁₂ alkyl), —NH(C₁-C₁₂ alkyl), —N(C₁-C₁₂ alkyl)₂, —S(C₁-C₁₂ alkyl), —S(C₆-C₁₀ aryl), —P(C₆-C₁₀ aryl)₃, —OP(═O)O—)O—(C₁-C₁₂ alkylene)-NMe₃⁺, C₁-C₁₂ alkyl, C₂-C₁₂ alkenyl, C₂-C₁₂ alkynyl, C₃-C₈ cycloalkyl, 3- to 8-membered heterocycloalkyl, C₆-C₁₀ aryl, or 5- to 10-membered heteroaryl is optionally substituted with one or more R^{La}; or
two R^{L′}, together with the one or more intervening atoms, form C₃-C₈ cycloalkyl, 3- to 8-membered heterocycloalkyl, C₆-C₁₅ aryl, or 5- to 15-membered heteroaryl, wherein the C₃-C₈ cycloalkyl, 3- to 8-membered heterocycloalkyl, C₆-C₁₅ aryl, or 5- to 15-membered heteroaryl is optionally substituted with one or more R^{La}; and
each R^{La} independently is oxo, halogen, —OH, —O(C₁-C₁₂ alkyl), —SH, —S(C₁-C₁₂ alkyl), —NH₂, —NH(C₁-C₁₂ alkyl), —N(C₁-C₁₂ alkyl)₂, C₁-C₁₂ alkyl, C₂-C₁₂ alkenyl, C₂-C₁₂ alkynyl, C₃-C₈ cycloalkyl, 3- to 8-membered heterocycloalkyl optionally substituted with one or more C₁-C₁₂ alkyl, C₆-C₁₀ aryl, or 5- to 10-membered heteroaryl; or
two R^{La}, together with the one or more intervening atoms, form C₃-C₈ cycloalkyl, 3- to 8-membered heterocycloalkyl, C₆-C₁₀ aryl, or 5- to 10-membered heteroaryl.

In some embodiments, L is —C(═O)R^{L}. In some embodiments, L is —C(═S)R^{L}.

In some embodiments, R^{L} is a C₂-C₃₅ hydrocarbon chain. In some embodiments, R^{L} is a C₁₃-C₂₁ hydrocarbon chain. In some embodiments, R^{L} is a C₁₉-C₂₇ hydrocarbon chain.

In some embodiments, R^{L} is a 2- to 35-membered hetero-hydrocarbon chain. In some embodiments, R^{L} is a 13- to 21-membered hetero-hydrocarbon chain. In some embodiments, R^{L} is a 19- to 27-membered hetero-hydrocarbon chain.

In some embodiments, R^{L} is a C₄-C₃₃ hydrocarbon chain.

In some embodiments, R^{L} is a C₉-C₂₁ hydrocarbon chain, a C₉-C₂₀ hydrocarbon chain, a C₉-C₁₉ hydrocarbon chain, a C₉-C₁₈ hydrocarbon chain, a C₉-C₁₇ hydrocarbon chain, a C₉-C₁₆ hydrocarbon chain, a C₉-C₁₅ hydrocarbon chain, or a C₉-C₁₄ hydrocarbon chain, wherein the hydrocarbon chain is optionally substituted with one or more R^{L′}.

In some embodiments, R^{L} is a C₁₃-C₂₁ hydrocarbon chain, a C₁₃-C₂₀ hydrocarbon chain, a C₁₃-C₁₉ hydrocarbon chain, a C₁₃-C₁₈ hydrocarbon chain, a C₁₃-C₁₇ hydrocarbon chain, a C₁₃-C₁₆ hydrocarbon chain, a C₁₃-C₁₅ hydrocarbon chain, or a C₁₃-C₁₄ hydrocarbon chain, wherein the hydrocarbon chain is optionally substituted with one or more R^{L′}.

In some embodiments, R^{L} is a C₁₀-C₂₁ hydrocarbon chain, a C₁₁-C₂₁ hydrocarbon chain, a C₁₂-C₂₁ hydrocarbon chain, or a C₁₃-C₂₁ hydrocarbon chain, wherein the hydrocarbon chain is optionally substituted with one or more R^{L′}.

In some embodiments, R^{L} is a C₁₄-C₂₁ hydrocarbon chain, a C₁₅-C₂₁ hydrocarbon chain, a C₁₆-C₂₁ hydrocarbon chain, a C₁₇-C₂₁ hydrocarbon chain, a C₁₈-C₂₁ hydrocarbon chain, a C₁₉-C₂₁ hydrocarbon chain, or a C₂₀-C₂₁ hydrocarbon chain, wherein the hydrocarbon chain is optionally substituted with one or more R^{L′}.

In some embodiments, R^{L} is a C₁₀-C₂₀ hydrocarbon chain, a C₁₁-C₁₉ hydrocarbon chain, a C₁₂-C₁₈ hydrocarbon chain, a C₁₃-C₁₇ hydrocarbon chain, or a C₁₄-C₁₆ hydrocarbon chain, wherein the hydrocarbon chain is optionally substituted with one or more R^{L′}.

In some embodiments, R^{L} is a C₁₄-C₂₀ hydrocarbon chain, a C₁₅-C₁₉ hydrocarbon chain, or a C₁₆-C₁₈ hydrocarbon chain, wherein the hydrocarbon chain is optionally substituted with one or more R^{L′}.

In some embodiments, R^{L} is a C₁₃-C₂₀ hydrocarbon chain or a C₁₄-C₁₉ hydrocarbon chain, wherein the hydrocarbon chain is optionally substituted with one or more R^{L′}.

In some embodiments, R^{L} is a 4- to 33-membered hetero-hydrocarbon chain.

In some embodiments, R^{L} is a 9- to 21-membered hetero-hydrocarbon chain, a 9- to 20-membered hetero-hydrocarbon chain, a 9- to 19-membered hetero-hydrocarbon chain, a 9- to 18-membered hetero-hydrocarbon chain, a 9- to 17-membered hetero-hydrocarbon chain, a 9- to 16-membered hetero-hydrocarbon chain, a 9- to 15-membered hetero-hydrocarbon chain, or a 9- to 14-membered hetero-hydrocarbon chain, wherein the hetero-hydrocarbon chain is optionally substituted with one or more R^{L′}.

In some embodiments, R^{L} is a 13- to 21-membered hetero-hydrocarbon chain, a 13- to 20-membered hetero-hydrocarbon chain, a 13- to 19-membered hetero-hydrocarbon chain, a 13- to 18-membered hetero-hydrocarbon chain, a 13- to 17-membered hetero-hydrocarbon chain, a 13- to 16-membered hetero-hydrocarbon chain, a 13- to 15-membered hetero-hydrocarbon chain, or a 13- to 14-membered hetero-hydrocarbon chain, wherein the hetero-hydrocarbon chain is optionally substituted with one or more R^{L′}.

In some embodiments, R^{L} is a 10- to 21-membered hetero-hydrocarbon chain, a 11- to 21-membered hetero-hydrocarbon chain, a 12- to 21-membered hetero-hydrocarbon chain, or a 13- to 21-membered hetero-hydrocarbon chain, wherein the hetero-hydrocarbon chain is optionally substituted with one or more R^{L′}.

In some embodiments, R^{L} is a 14- to 21-membered hetero-hydrocarbon chain, a 15- to 21-membered hetero-hydrocarbon chain, a 16- to 21-membered hetero-hydrocarbon chain, a 17- to 21-membered hetero-hydrocarbon chain, a 18- to 21-membered hetero-hydrocarbon chain, a 19- to 21-membered hetero-hydrocarbon chain, or a 20- to 21-membered hetero-hydrocarbon chain, wherein the hetero-hydrocarbon chain is optionally substituted with one or more R^{L′}.

In some embodiments, R^{L} is a 10- to 20-membered hetero-hydrocarbon chain, a 11- to 19-membered hetero-hydrocarbon chain, a 12- to 18-membered hetero-hydrocarbon chain, a 13- to 17-membered hetero-hydrocarbon chain, or a 14- to 16-membered hetero-hydrocarbon chain, wherein the hetero-hydrocarbon chain is optionally substituted with one or more R^{L′}.

In some embodiments, R^{L} is a 14- to 20-membered hetero-hydrocarbon chain, a 15- to 19-membered hetero-hydrocarbon chain, or a 16- to 18-membered hetero-hydrocarbon chain, wherein the hetero-hydrocarbon chain is optionally substituted with one or more R^{L′}.

In some embodiments, R^{L} is a 13- to 20-membered hetero-hydrocarbon chain or a 14- to 19-membered hetero-hydrocarbon chain optionally substituted with one or more R^{L′}.

In some embodiments, L is —C(═O)R^{L} or —C(═S)R^{L};
R^{L} is C₂-C₃₅ hydrocarbon chain or 2- to 35-membered hetero-hydrocarbon chain, wherein the C₂-C₃₅ hydrocarbon chain or 2- to 35-membered hetero-hydrocarbon chain is optionally substituted with one or more R^{L′};
each R^{L′} independently is oxo, dihalocarbene, cyano, halogen, —OH, —COOH, —C(O)NHOH, —SO₃H, —S(C₁-C₁₂ alkyl), —S(C₆-C₁₀ aryl), —P(C₆-C₁₀ aryl)₃, or C₆-C₁₀ aryl, wherein the —S(C₁-C₁₂ alkyl), —S(C₆-C₁₀ aryl), —P(C₆-C₁₀ aryl)₃, or C₆-C₁₀ aryl is optionally substituted with one or more R^{La}; or
two R^{L′}, together with the one or more intervening atoms, form C₃-C₈ cycloalkyl, 3- to 8-membered heterocycloalkyl, or 5- to 15-membered heteroaryl, wherein the C₃-C₈ cycloalkyl, 3- to 8-membered heterocycloalkyl, or 5- to 15-membered heteroaryl is optionally substituted with one or more R^{La}; and
each R^{La} independently is halogen or C₁-C₁₂ alkyl.

In some embodiments, at least one R^{L′} is oxo.

In some embodiments, at least one R^{L′} is difluorocarbene.

In some embodiments, at least one R^{L′} is cyano.

In some embodiments, at least one R^{L′} is halogen (e.g., F, Cl, or Br).

In some embodiments, at least one R^{L′} is —OH.

In some embodiments, at least one R^{L′} is —COOH.

In some embodiments, at least one R^{L′} is —COO(C₁-C₁₂ alkyl) optionally substituted with one or more R^{La}.

In some embodiments, at least one R^{L′} is —CO(C₁-C₃₀ alkyl) optionally substituted with one or more R^{La}.

In some embodiments, at least one R^{L′} is —NHCO(C₁-C₃₀ alkyl) optionally substituted with one or more R^{La}.

In some embodiments, at least one R^{L′} is —C(O)NHOH.

In some embodiments, at least one R^{L′} is —SO₃H.

In some embodiments, at least one R^{L′} is —S(C₁-C₁₂ alkyl) optionally substituted with one or more R^{La}.

In some embodiments, at least one R^{L′} is —S(C₆-C₁₀ aryl) optionally substituted with one or more R^{La}.

In some embodiments, at least one R^{L′} is

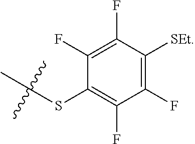

In some embodiments, at least one R^{L′} is —P(C₆-C₁₀ aryl)₃ optionally substituted with one or more R^{La}.

In some embodiments, at least one R^{L′} is —OP(═O)(O)O—(C₁-C₁₂ alkylene)-NMe₃⁺ optionally substituted with one or more R^{La}.

In some embodiments, at least one R^{L′} is —OP(═O)(O⁻)O—(C₂ alkylene)-NMe₃⁺.

In some embodiments, at least one R^{L} is C₆-C₁₀ aryl optionally substituted with one or more R^{La}.

In some embodiments, at least one R^{L′} is 5- to 10-membered heteroaryl optionally substituted with one or more R^{La}.

In some embodiments, two R^{L′}, together with the one or more intervening atoms, form C₃-C₈ cycloalkyl optionally substituted with one or more R^{La}.

In some embodiments, two R^{L′}, together with the one or more intervening atoms, form

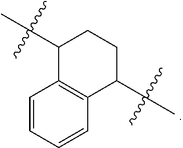 [SMILES: *C1CCC(*)c2ccccc21]

In some embodiments, two R^{L′}, together with the one or more intervening atoms, form 3- to 8-membered heterocycloalkyl optionally substituted with one or more R^{La}.

In some embodiments, two R^{L′}, together with the one or more intervening atoms, form

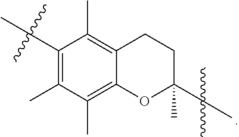 [SMILES: *c1c(C)c(C)c2c(c1C)CC[C@](*)(C)O2]

In some embodiments, two R^{L′}, together with the one or more intervening atoms, form

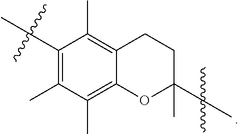 [SMILES: *c1c(C)c(C)c2c(c1C)CCC(*)(C)O2]

In some embodiments, two R^{L′}, together with the one or more intervening atoms, form 5- to 15-membered heteroaryl optionally substituted with one or more R^{La}.

In some embodiments, two R^{L′}, together with the one or more intervening atoms, form

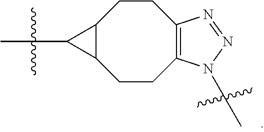 [SMILES: *C1C2CCc3nnn(*)c3CCC12]

In some embodiments, two R^{L′}, together with the one or more intervening atoms, form

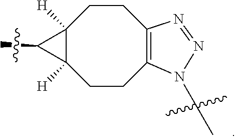

In some embodiments, at least one R^{La} is halogen (e.g., F, Cl, or Br).

In some embodiments, at least one R^{La} is 5- to 10-membered heteroaryl.

In some embodiments, two R^{La}, together with the one or more intervening atoms, form C₃-C₈ cycloalkyl.

In some embodiments, two R^{La}, together with the one or more intervening atoms, form 3- to 8-membered heterocycloalkyl.

In some embodiments, B is a nucleobase moiety.

The term, “nucleobase moiety”, as used herein, refers to a nucleobase that is attached to the rest of the compound, e.g., via an atom of the nucleobase or a functional group thereof.

In some embodiments, the nucleobase moiety is adenine (A), cytosine (C), guanine (G), thymine (T), or uracil (U).

In some embodiments, the nucleobase moiety is

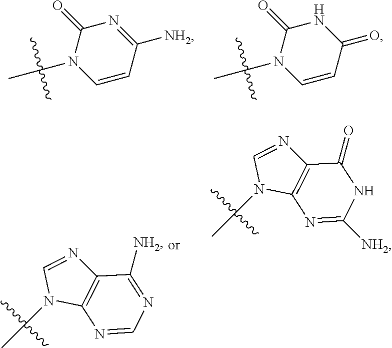 [SMILES: *n1ccc(N)nc1=O ; *n1ccc(=O)[nH]c1=O ; *n1cnc2c(N)ncnc21 ; *n1cnc2c(=O)[nH]c(N)nc21]

wherein [symbol] indicates the point of attachment to the rest of ligand.

In some embodiments, the nucleobase moiety is a modified nucleobase.

In some embodiments, the modified nucleobase is 5-methylcytosine.

In some embodiments, the modified nucleobase is hypoxanthine, xanthine, or 7-methylguanine.

In some embodiments, the modified nucleobase is 5,6-dihydrouracil, 5-methylcytosine, or 5-hydroxymethylcytosine.

In some embodiments, the nucleobase moiety is an artificial nucleobase.

In some embodiments, the artificial nucleobase is isoguanine, isocytosine, 2-amino-6-(2-thienyl)purine, or pyrrole-2-carbaldehyde.

In some embodiments, the targeting ligand comprises a structure selected from the structures described in Table 1, wherein:
each of # and ## independently is an attachment to the rest of the conjugate, or
when the targeting ligand is at the 3′-terminus of the oligonucleotide, then: # is an attachment to the rest of the conjugate; and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano; or
when the targeting ligand is at the 5′-terminus of the oligonucleotide, then: # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

TABLE 1
Structure of Targeting Ligands

| No. | Structure |
|---|---|
| N-1 | 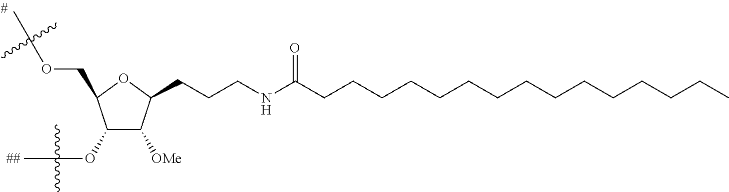 |
| N-3 | 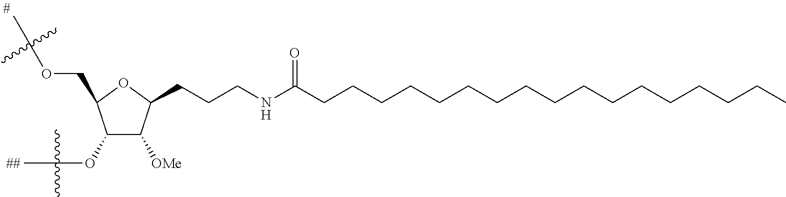 |
| N-5 | 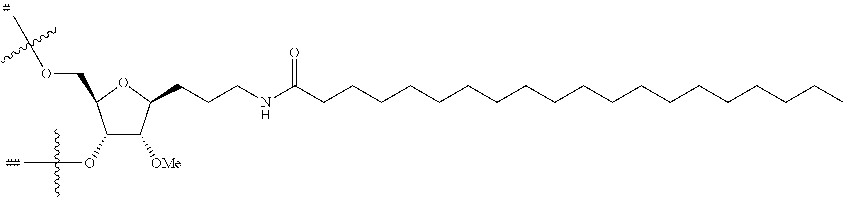 |
| N-7 | 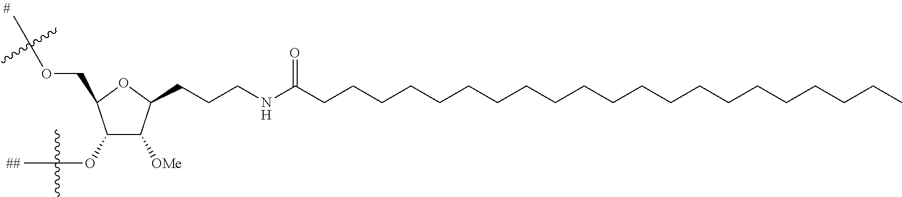 |
| N-9 | 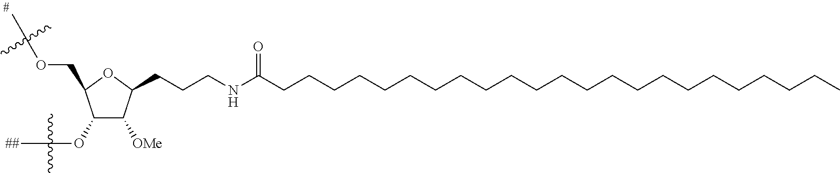 |
| N-11 | 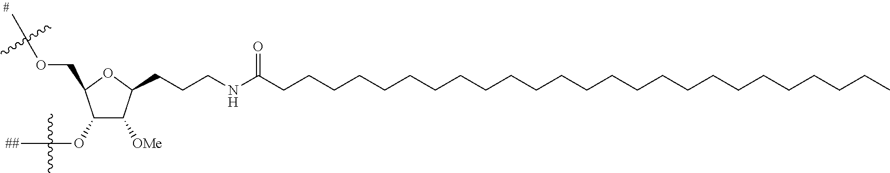 |
| N-13 | 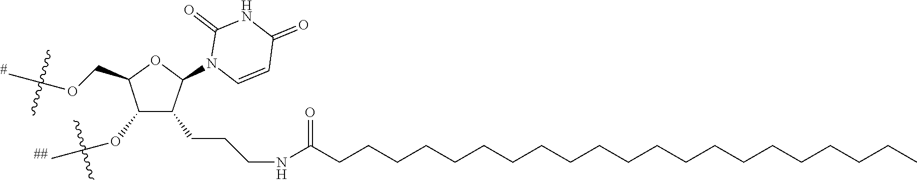 [SMILES: *OC[C@H]1O[C@@H](n2ccc(=O)[nH]c2=O)[C@H](CCCNC(=O)CCCCCCCCCCCCCCCCCCCCC)[C@@H]1O*] |
| N-15 | 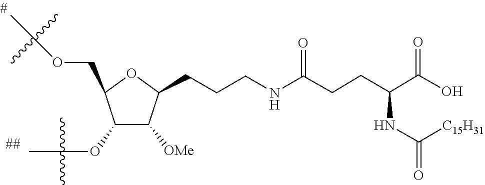 |
| N-17 | 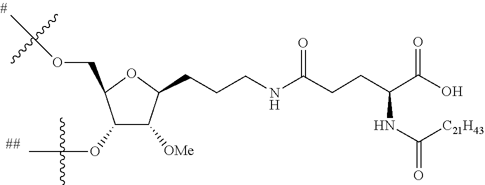 |
| N-19 | 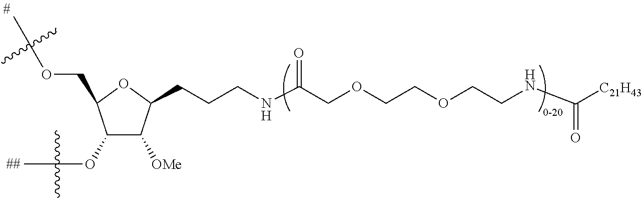 |
| N-21 | 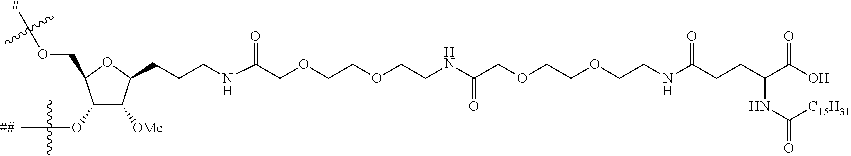 |
| N-23 | 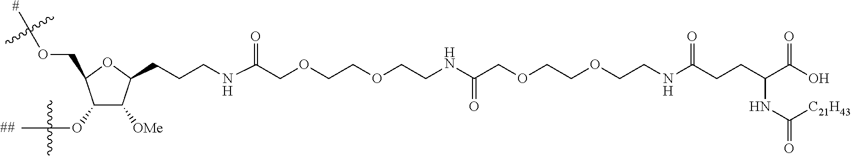 |
| N-25 | 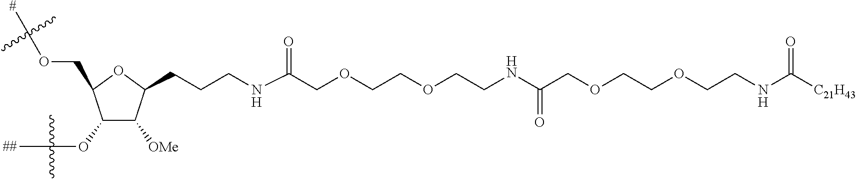 |
| N-27 | 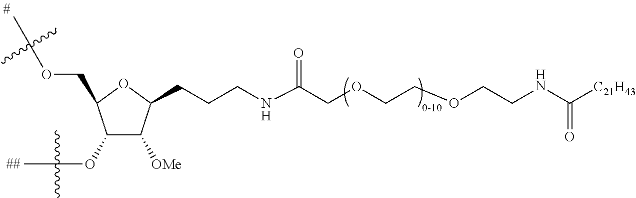 |
| N-29 | 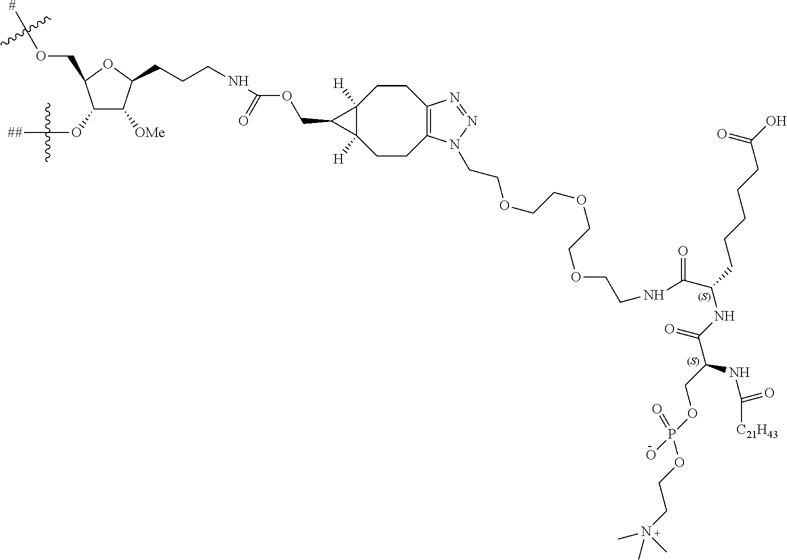 |
| N-31 | 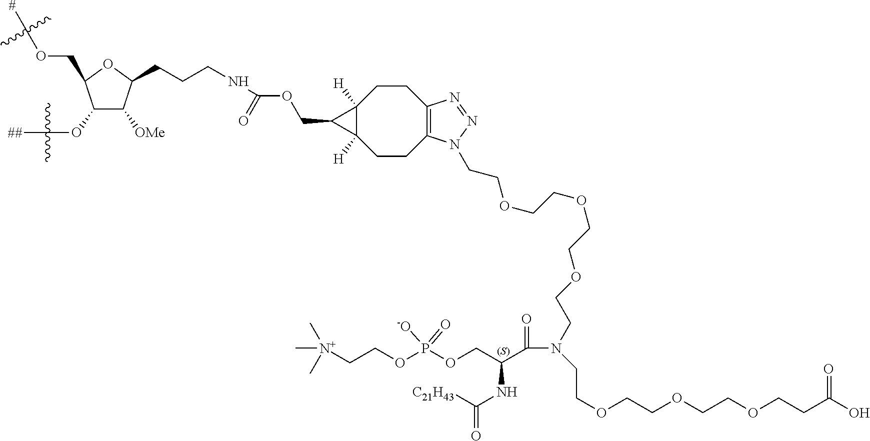 |
| N-33 | 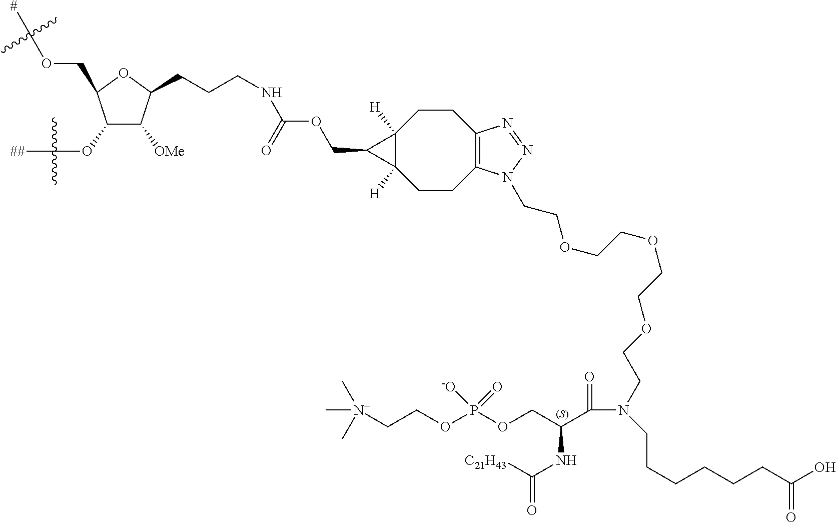 |
| N-33a | 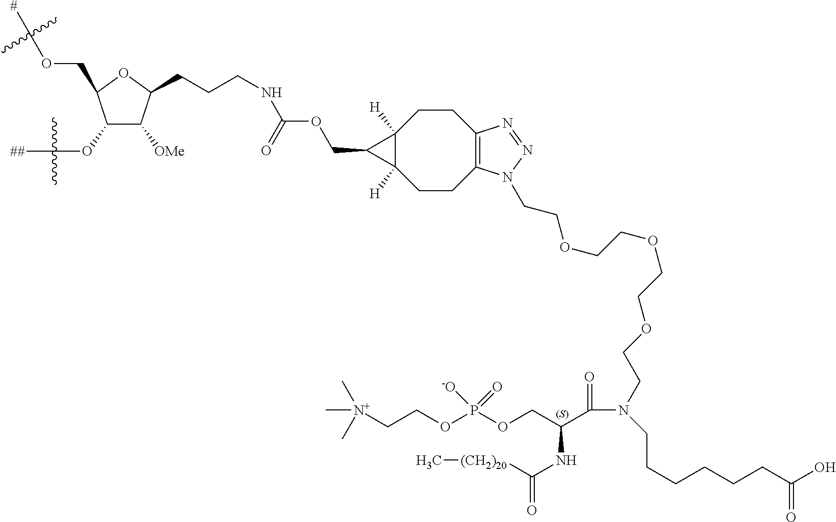 |
| N-35 | 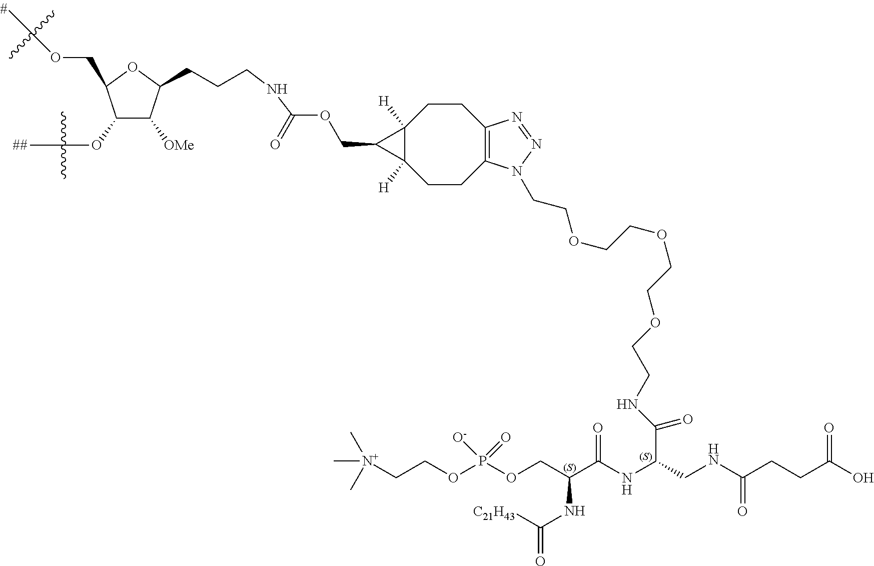 |
| N-37 | 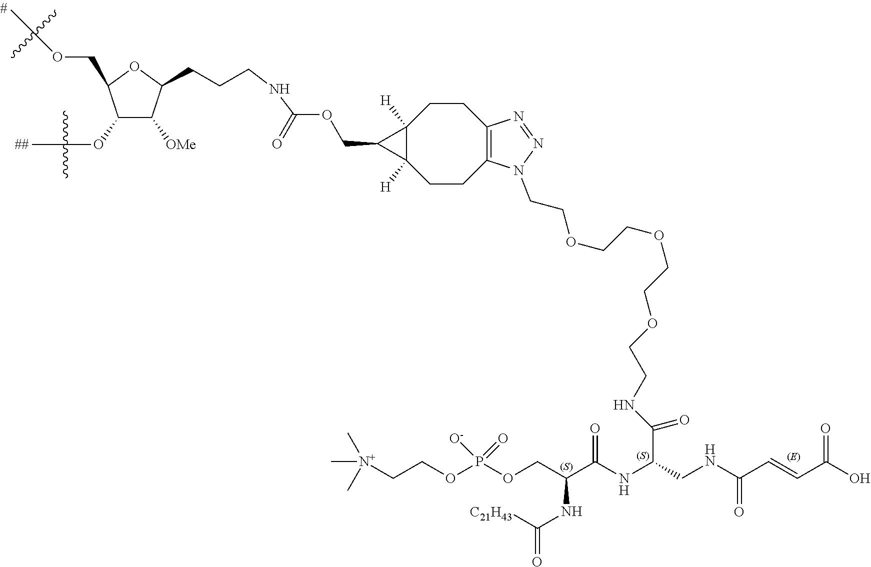 |
| N-39 | 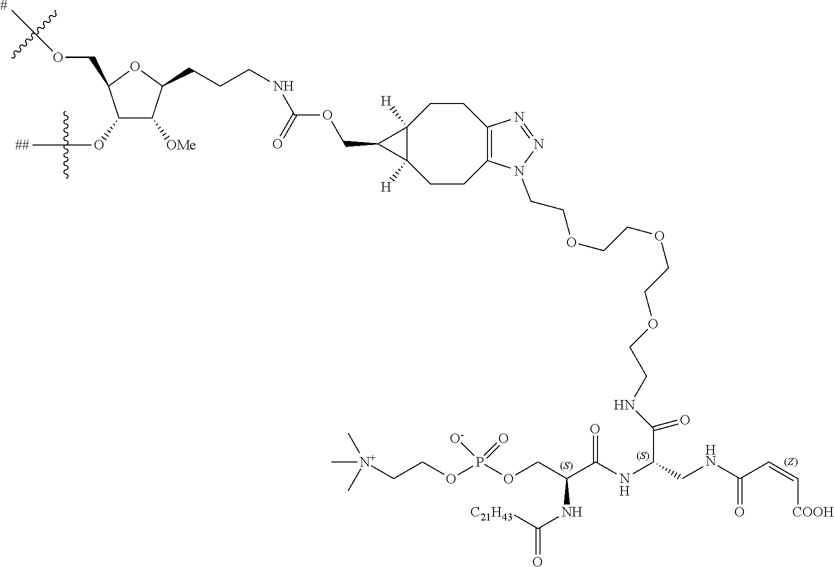 |
| N-41 | 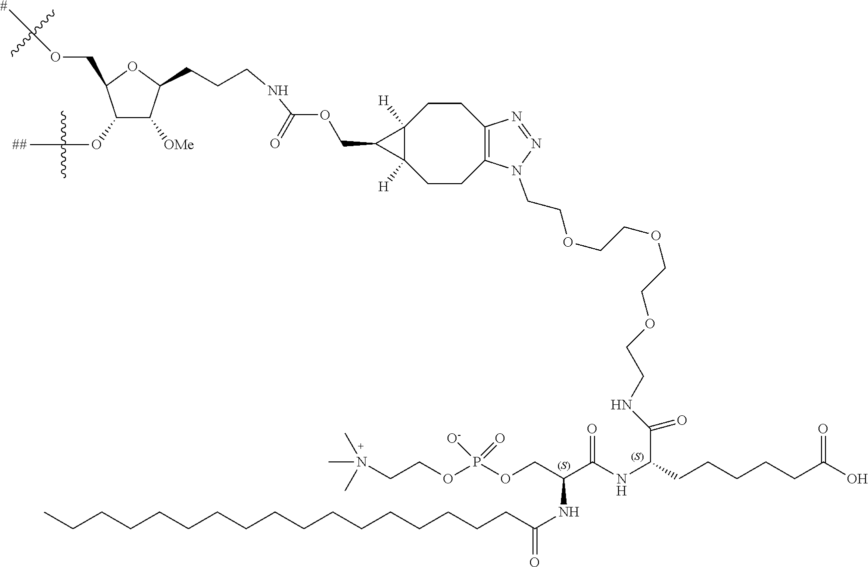 |
| N-43 | 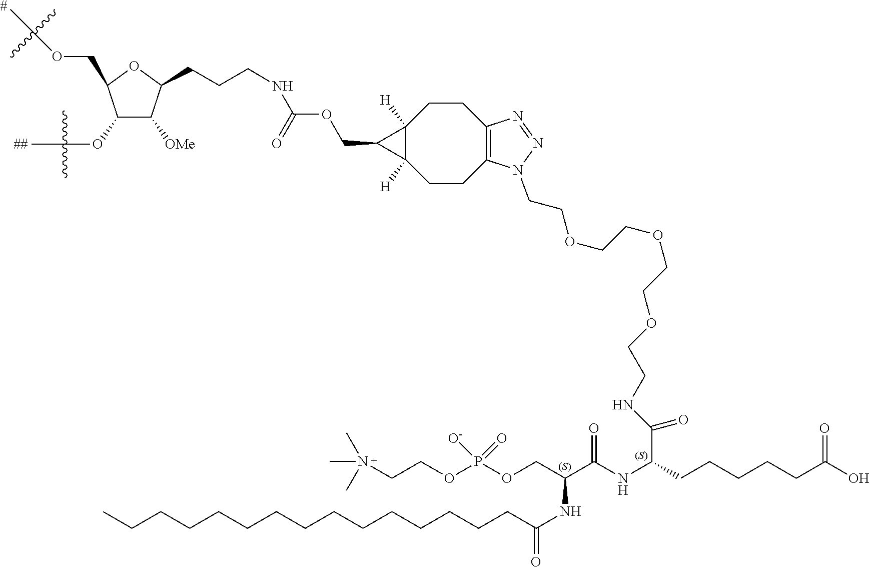 |
| N-45 | 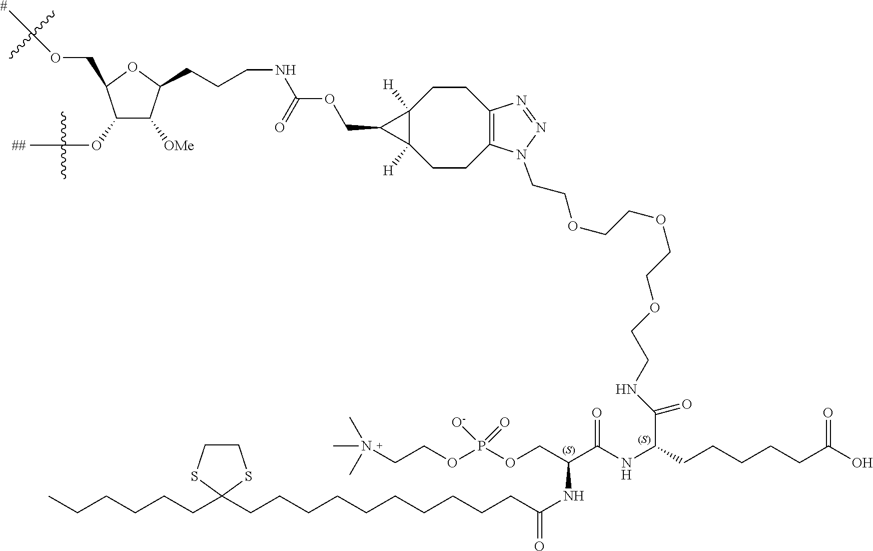 |
| N-47 | 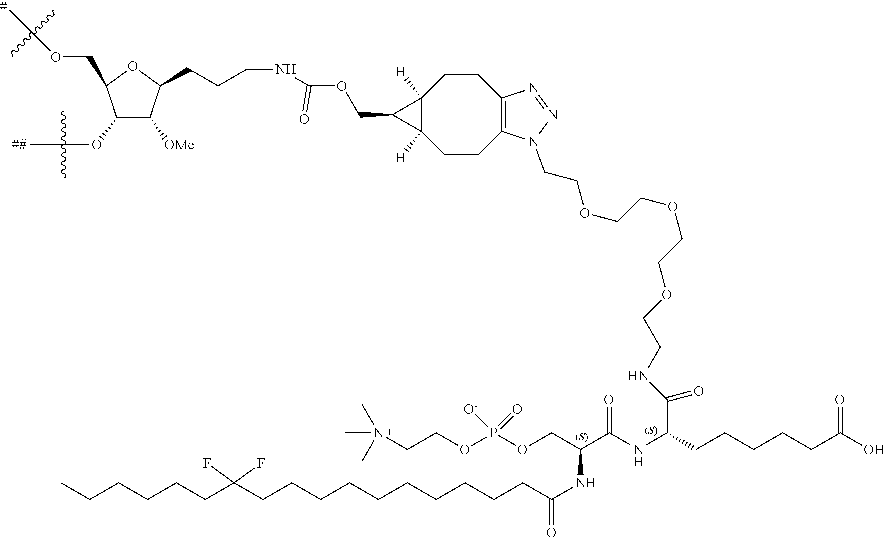 |
| N-49 | 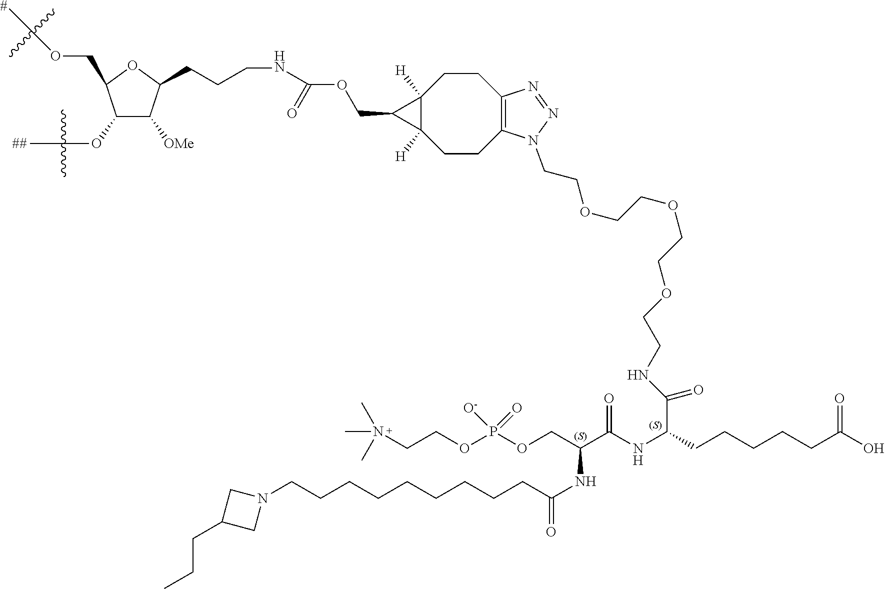 |
| N-51 | 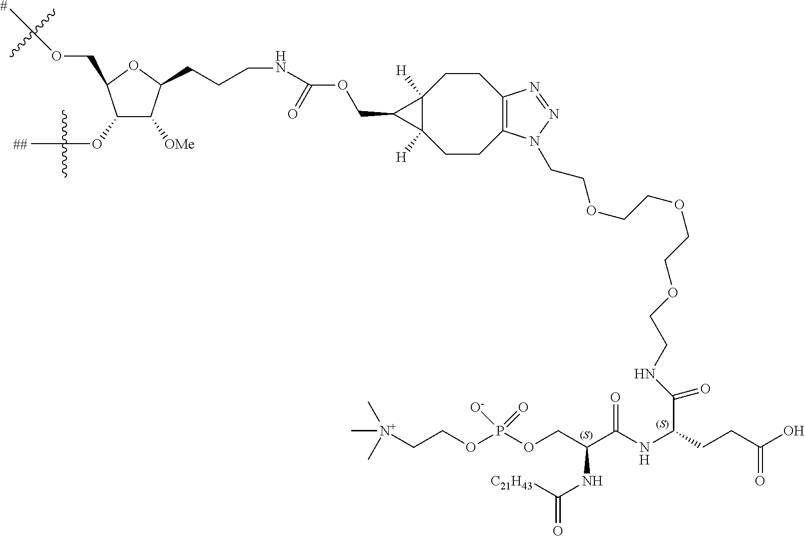 |
| N-53 | 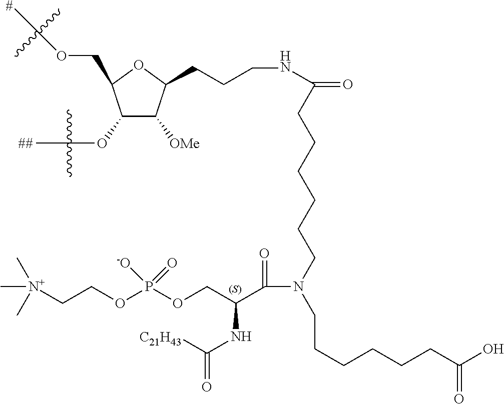 |
| N-55 | 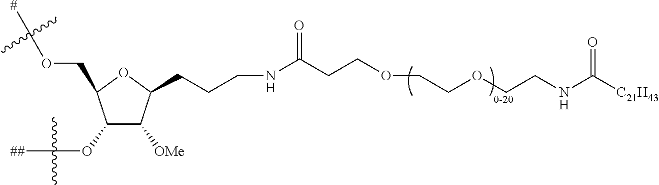 |
| N-57 | 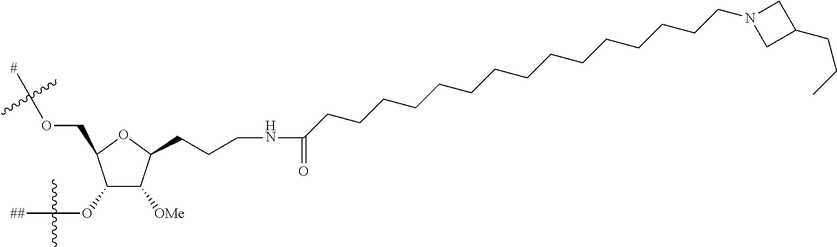 |
| N-59 | 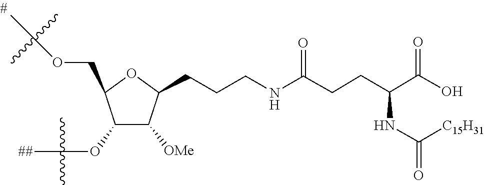 |
| N-61 | 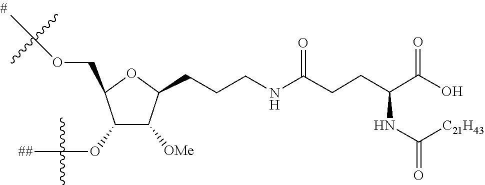 |
| N-63 | 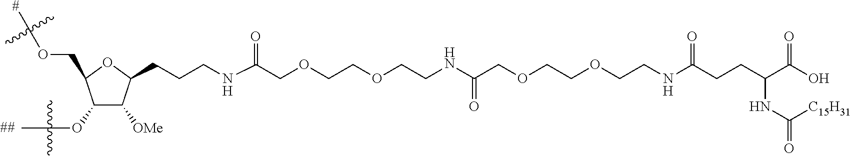 |
| N-65 | 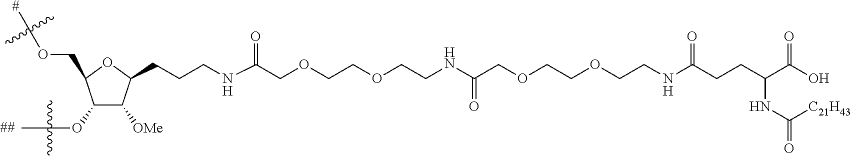 |
| N-67 | 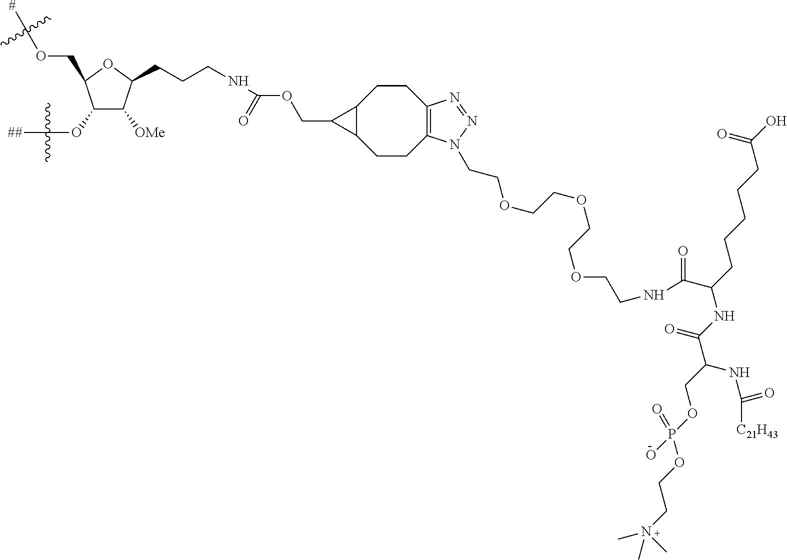 |
| N-69 | 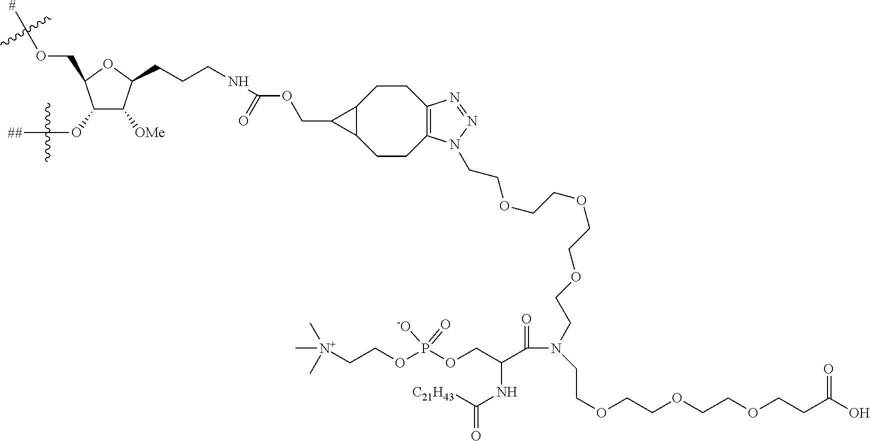 |
| N-71 | 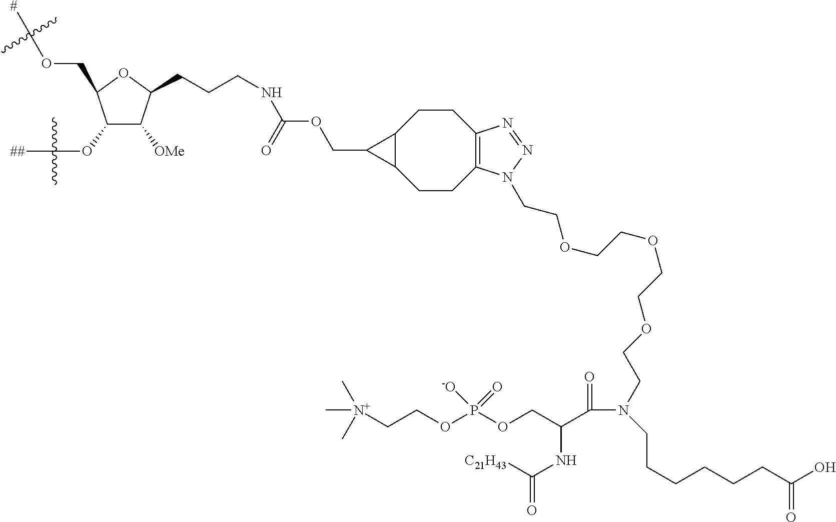 |
| N-73 | 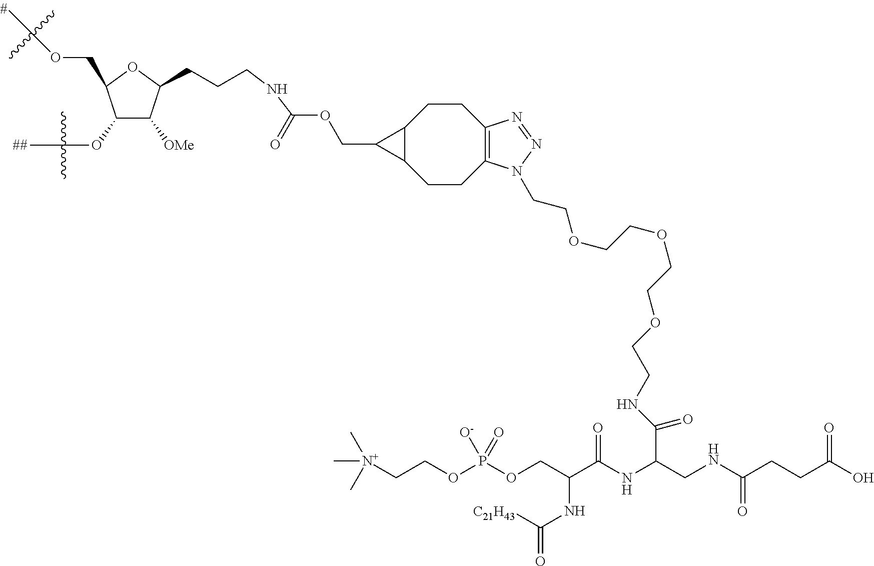 |
| N-75 | 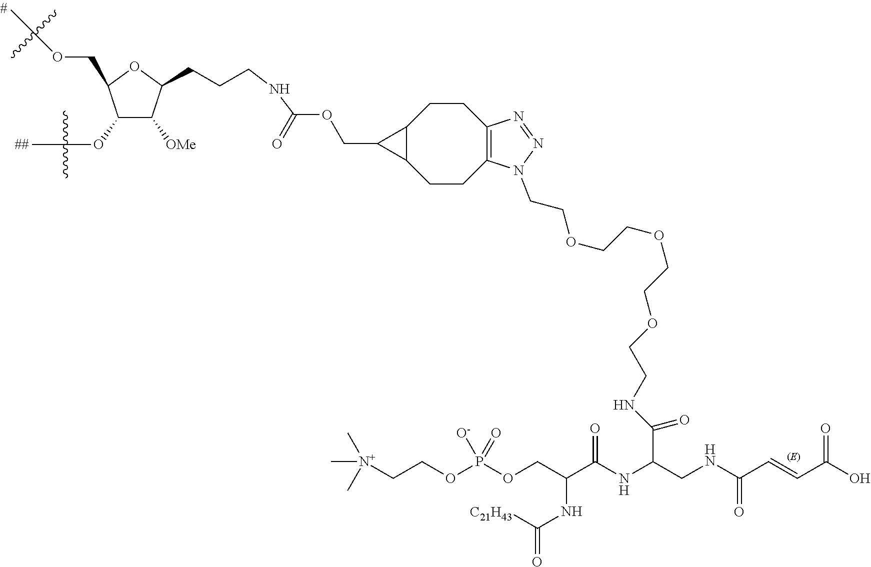 |
| N-77 | 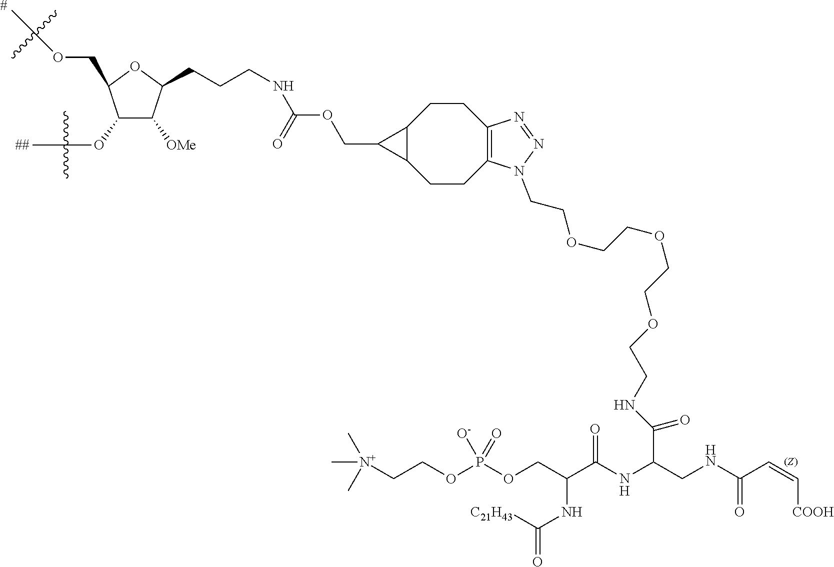 |
| N-79 | 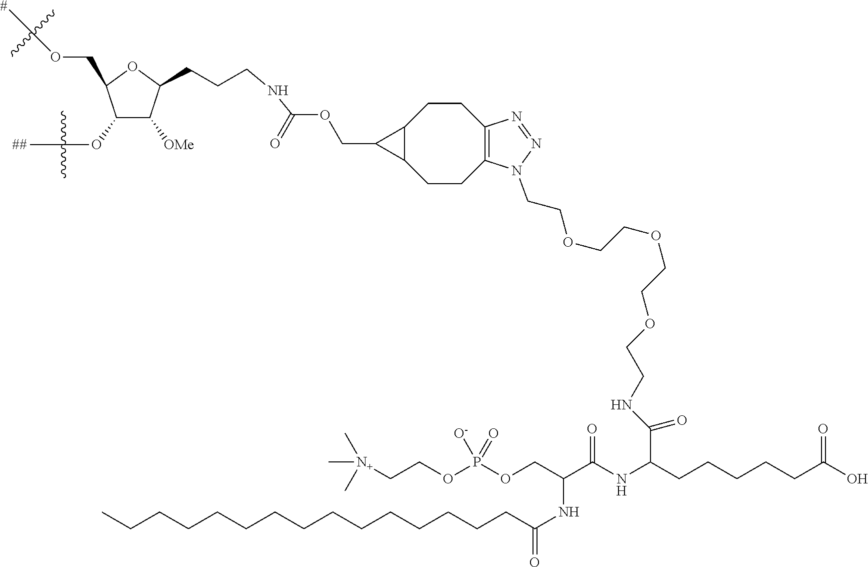 |
| N-81 | 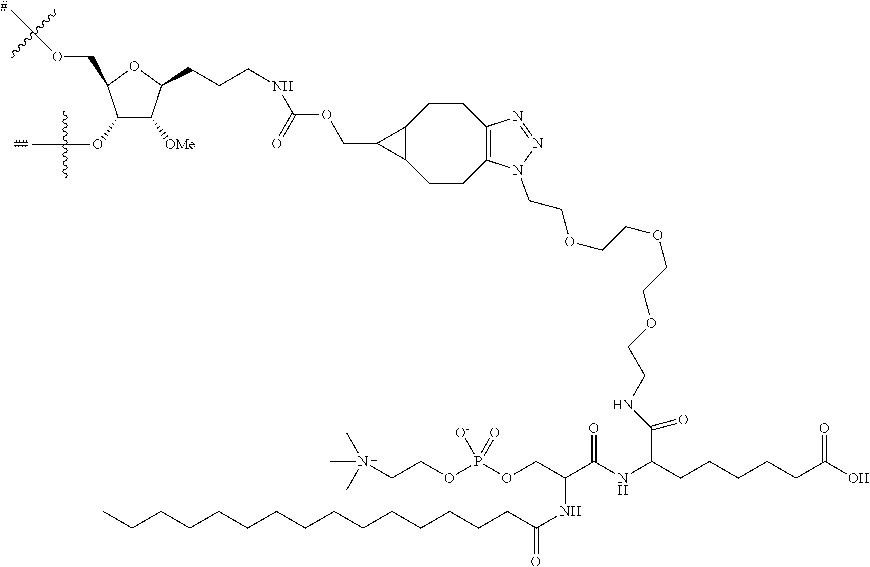 |
| N-83 | 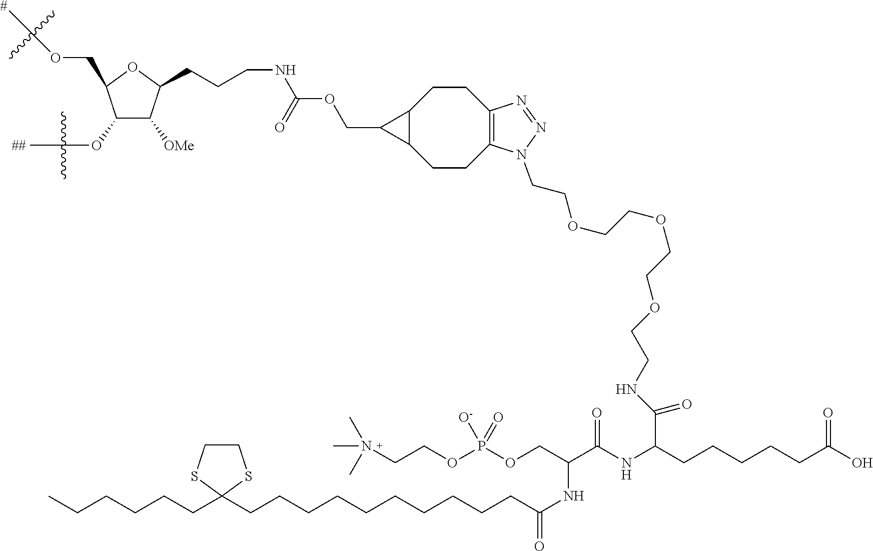 |
| N-85 | 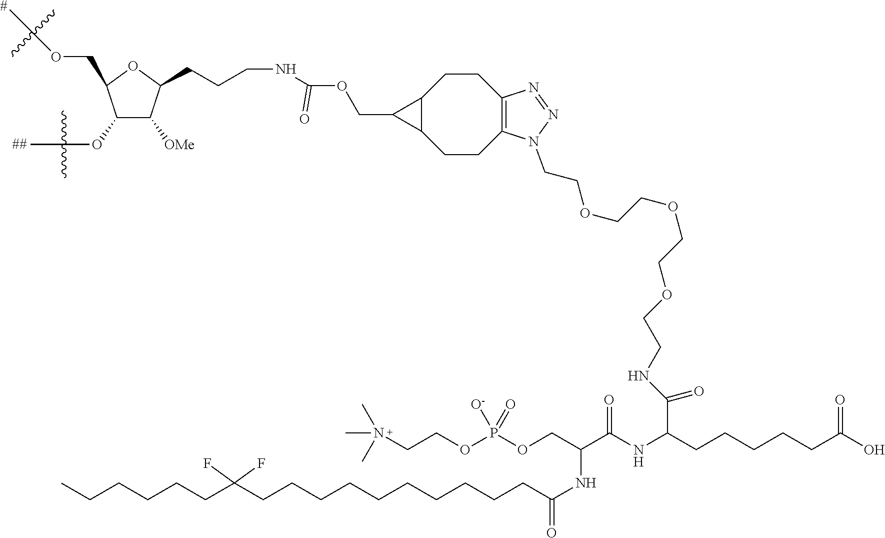 |
| N-87 | 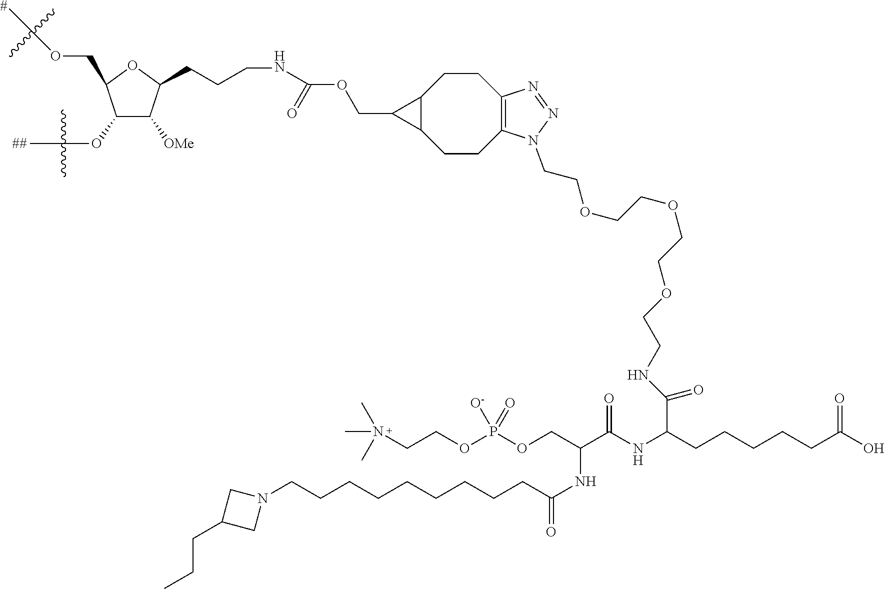 |
| N-89 | 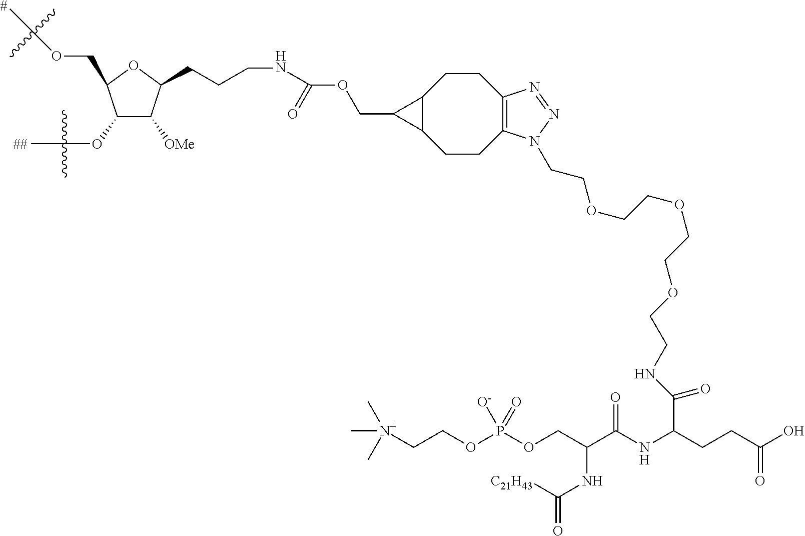 |
| N-91 | 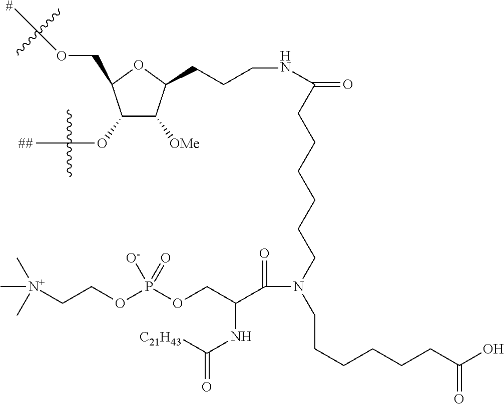 |

In some embodiments, the targeting ligand comprises a structure selected from the structures described in Table 1, wherein when the targeting ligand is at the 3′-terminus of the oligonucleotide, then # is an attachment to the rest of the conjugate; and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the targeting ligand comprises a structure selected from the structures described in Table 1, wherein when the targeting ligand is at the 3′-terminus of the oligonucleotide, then # is an attachment to the rest of the conjugate; and ## is H.

In some embodiments, the conjugate comprises a structure selected from the structures described in Table 1 at the 3′-terminus of the oligonucleotide, wherein # is an attachment to the rest of the conjugate, and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises a structure selected from the structures described in Table 1 at the 3′-terminus of the oligonucleotide, wherein # is an attachment to the rest of the conjugate, and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises a structure selected from the structures described in Table 1 at the 3′-terminus of the oligonucleotide, wherein # is an attachment to the rest of the conjugate, and ## is H.

In some embodiments, the targeting ligand comprises a structure selected from the structures described in Table 1, wherein the ## comprises a phosphate group (i.e., a terminal phosphate group:

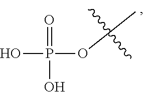 [SMILES: *OP(=O)(O)O]

or a non-terminal phosphate group:

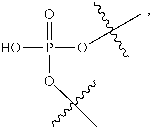 [SMILES: *OP(=O)(O)O*]

wherein each [symbol] independently is an attachment to the rest of the conjugate). In some embodiments, the phosphate group comprises a phosphodiester bond. In some embodiments, the phosphate group is a terminal group.

In some embodiments, the targeting ligand comprises a structure selected from the structures described in Table 1, wherein the ## comprises a thiophosphate group (i.e., a terminal thiophosphate group:

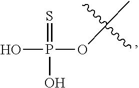 [SMILES: *OP(O)(O)=S]

or a non-terminal thiophosphate group:

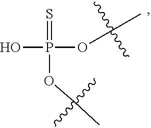 [SMILES: *OP(O)(=S)O*]

wherein each [symbol] independently is an attachment to the rest of the conjugate). In some embodiments, the thiophosphate group comprises a phosphorothioate bond. In some embodiments, the phosphorothioate group is a terminal group.

In some embodiments, C₂₁H₄₃ of the targeting ligand is H₃C—(CH₂)₂₀—.

In some embodiments, the targeting ligand comprises N-33.

In some embodiments, the targeting ligand comprises N-33, wherein when N-33 is at the 3′-terminus of the oligonucleotide, then # is an attachment to the rest of the conjugate; and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the targeting ligand comprises N-33, wherein when N-33 is at the 3′-terminus of the oligonucleotide, then # is an attachment to the rest of the conjugate; and ## is H.

In some embodiments, the conjugate comprises N-33 at the 3′-terminus of the oligonucleotide, wherein # is an attachment to the rest of the conjugate, and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises N-33 at the 3′-terminus of the oligonucleotide, wherein # is an attachment to the rest of the conjugate, and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises N-33 at the 3′-terminus of the oligonucleotide, wherein # is an attachment to the rest of the conjugate, and ## is H.

In some embodiments, the targeting ligand comprises G1b-BCN—C22PC2:

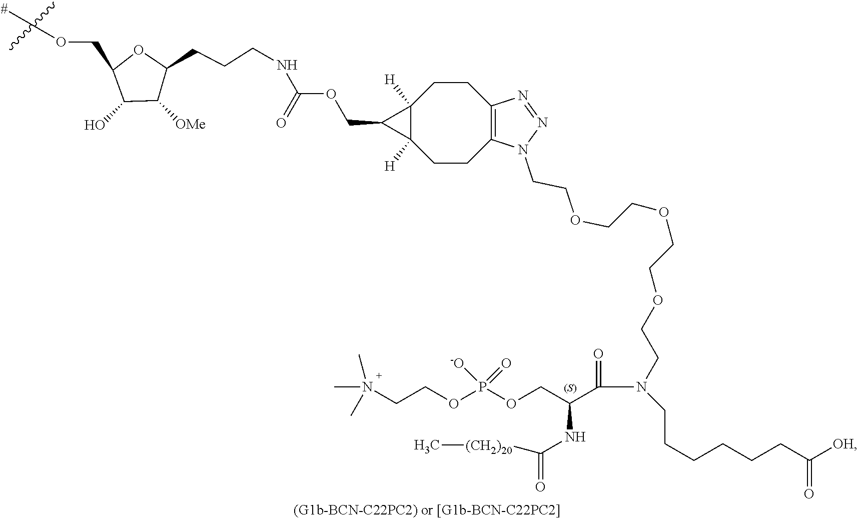

wherein # is an attachment to the rest of the conjugate.

In some embodiments, the conjugate comprises G1b-BCN—C22PC2 at the 3′-terminus of the oligonucleotide.

As used herein, “BCN” of G1b-BCN—C22PC2 refers to

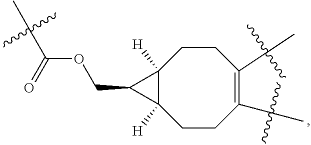 [SMILES: *C(=O)OC[C@@H]1[C@@H]2CC/C(*)=C(/*)CC[C@@H]21]

and “C22PC2” of G1b-BCN—C22PC2 refers to

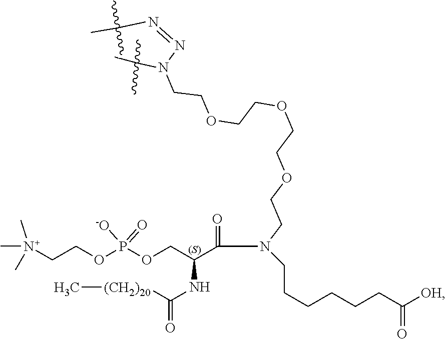

wherein each [symbol] independently is an attachment to the rest of the G1b-BCN—C22PC2.

In some embodiments, the targeting ligand comprises a structure selected from the structures described in Table 2, wherein:
each of # and ## independently is an attachment to the rest of the conjugate, or
when the targeting ligand is at the 3′-terminus of the oligonucleotide, then: # is an attachment to the rest of the conjugate; and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano; or
when the targeting ligand is at the 5′-terminus of the oligonucleotide, then: # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and ## is an attachment to the rest of the conjugate.

TABLE 2
Structures of Targeting Ligands

| No. | Structure |
|---|---|
| Q-1 | 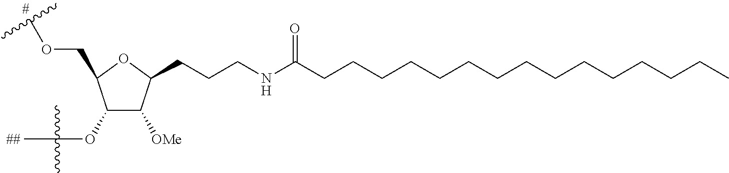 |
| Q-3 | 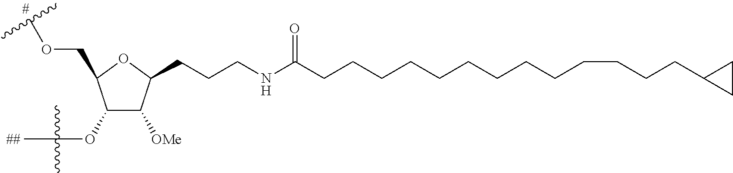 |
| Q-5 | 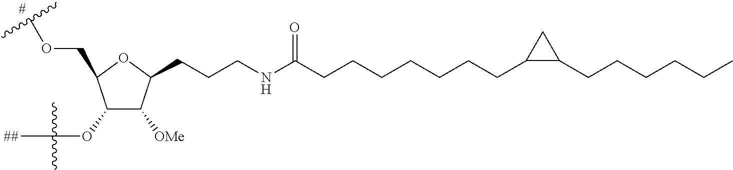 |
| Q-7 | 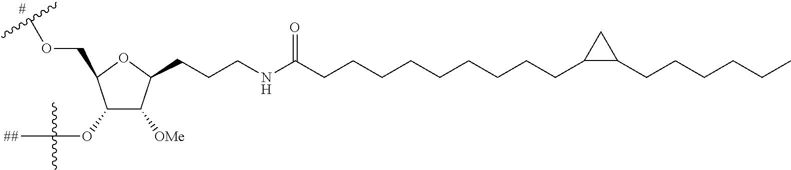 |
| Q-9 | 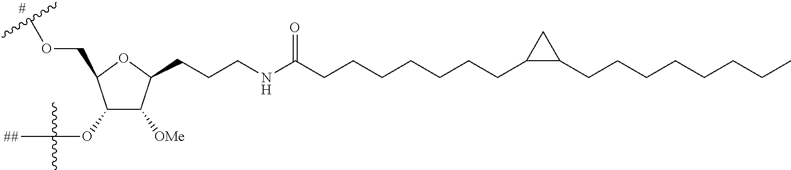 |
| Q-11 | 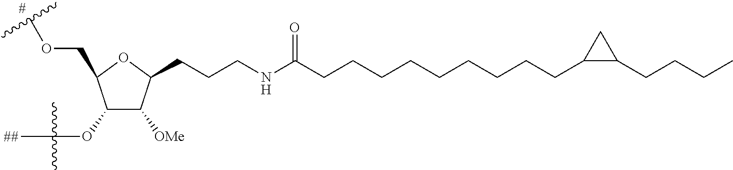 |
| Q-13 | 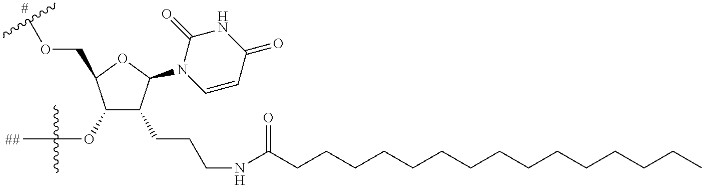 |
| Q-15 | 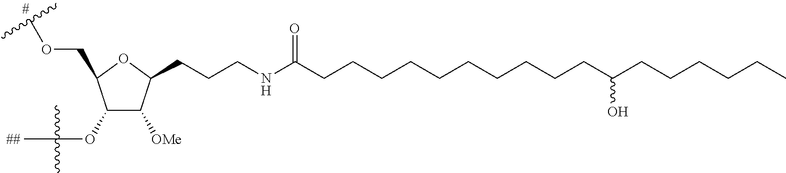 |
| Q-17 | 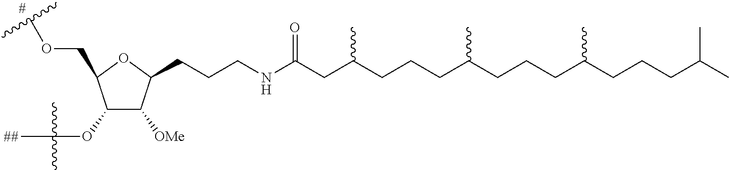 |
| Q-19 | 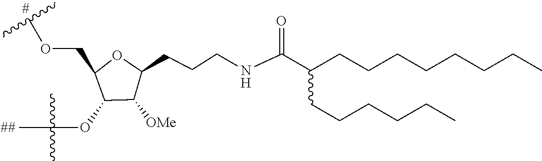 [SMILES: *OC[C@H]1O[C@@H](CCCNC(=O)C(CCCCCC)CCCCCCCC)[C@H](OC)[C@@H]1O*] |
| Q-21 | 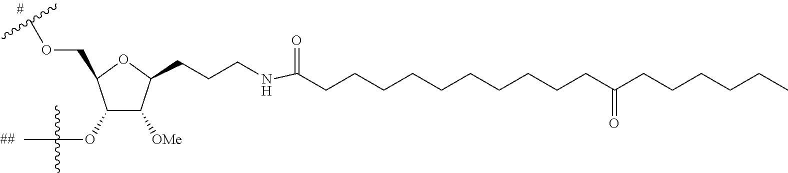 |
| Q-23 | 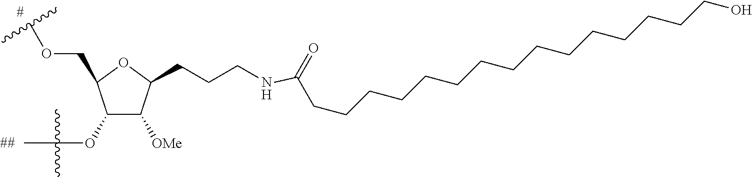 |
| Q-25 | 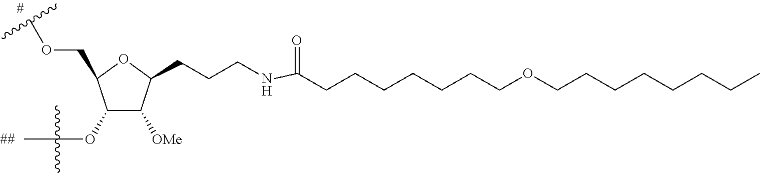 |
| Q-27 | 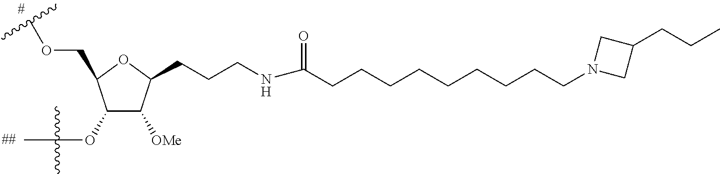 |
| Q-29 | 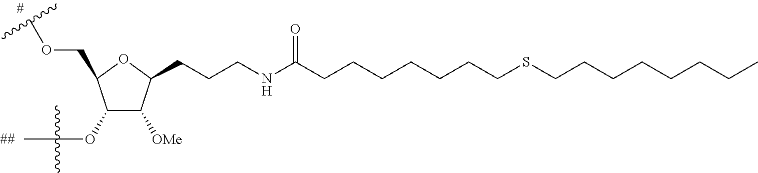 |
| Q-31 | 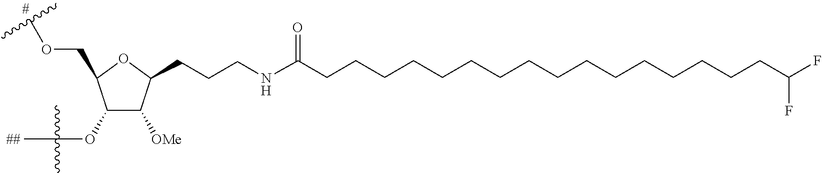 |
| Q-33 | 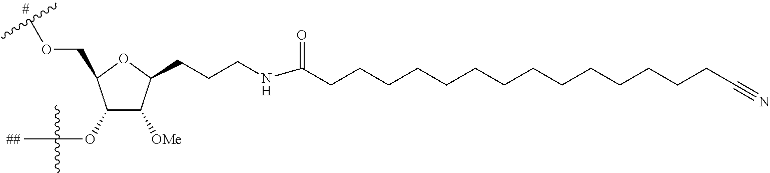 |
| Q-35 | 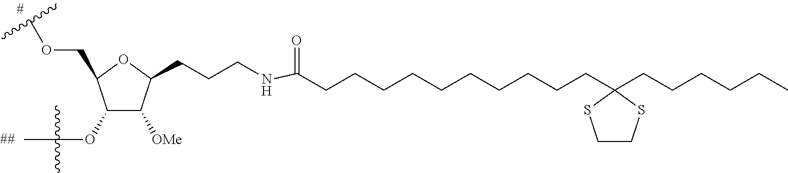 |
| Q-37 | 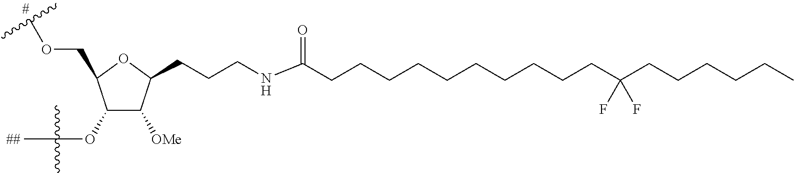 |
| Q-39 | 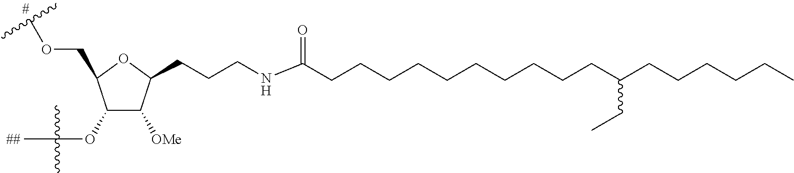 |
| Q-41 | 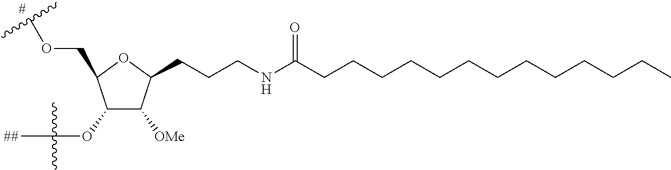 |
| Q-43 | 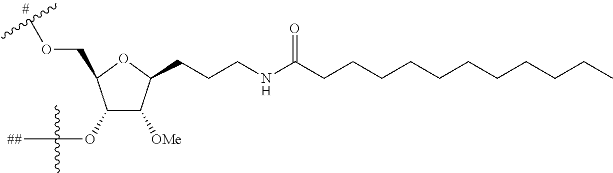 |
| Q-45 | 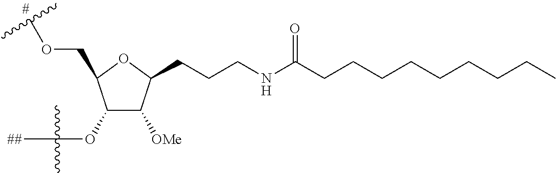 |
| Q-47 | 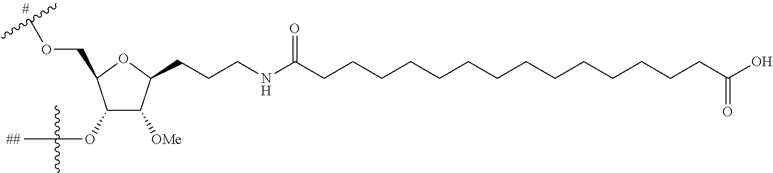 |
| Q-49 | 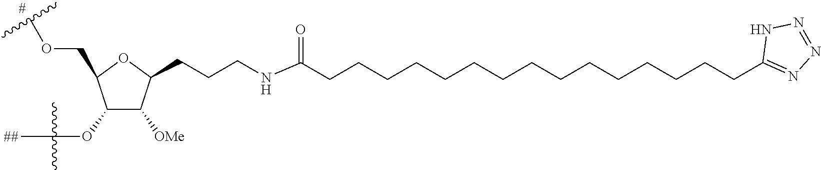 |
| Q-51 | 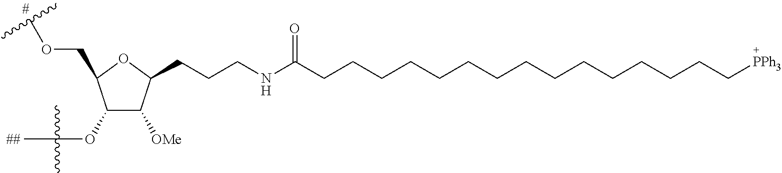 |
| Q-53 | 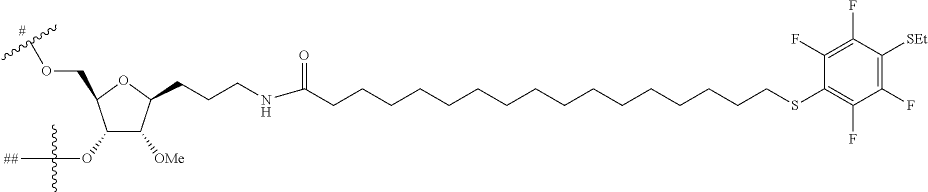 |
| Q-55 | 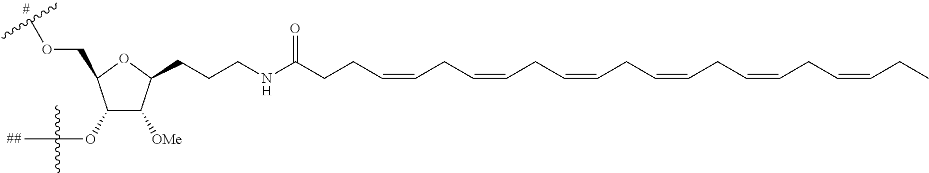 |
| Q-57 | 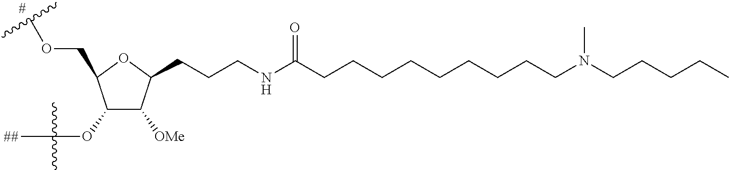 |
| Q-59 | 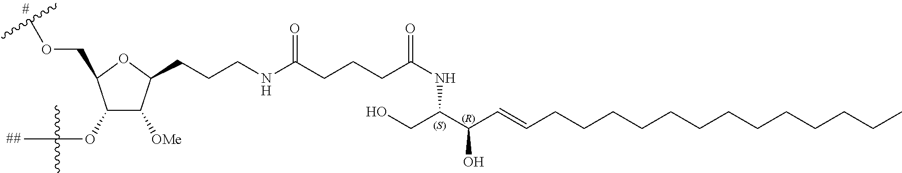 |
| Q-61 | 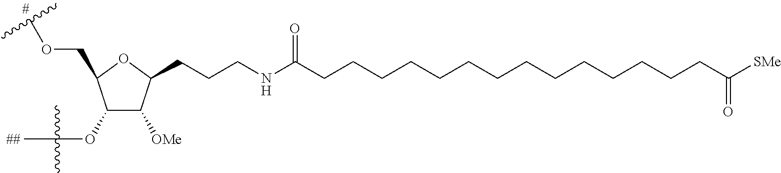 |
| Q-63 | 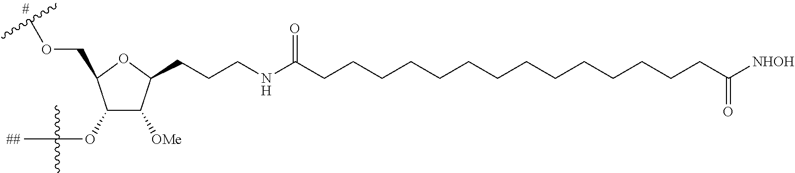 |
| Q-65 | 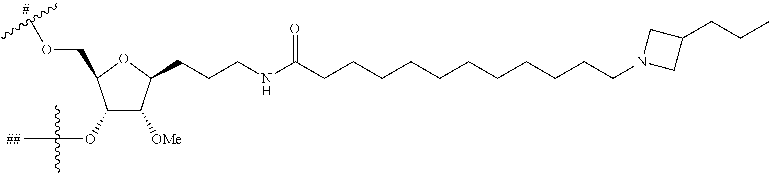 |
| Q-67 | 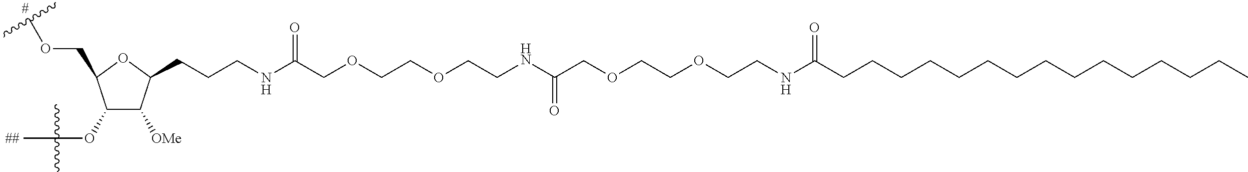 [SMILES: *OC[C@H]1O[C@@H](CCCNC(=O)COCCOCCNC(=O)COCCOCCNC(=O)CCCCCCCCCCCCCCC)[C@H](OC)[C@@H]1O*] |
| Q-69 | 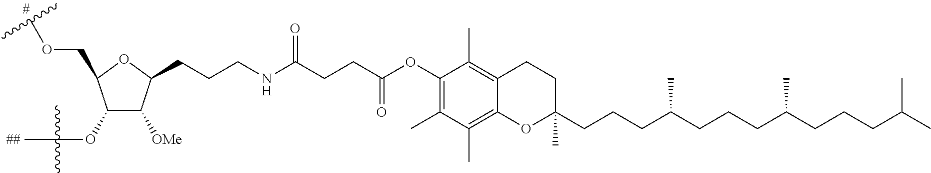 |
| Q-71 | 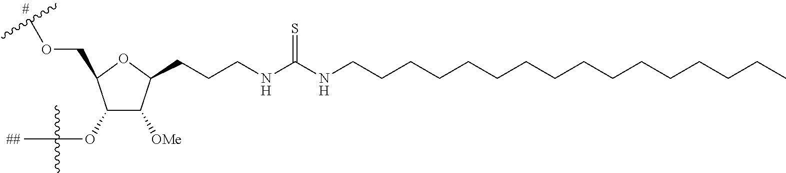 |
| Q-73 | 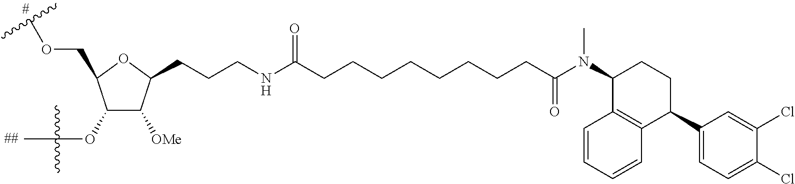 |
| Q-75 | 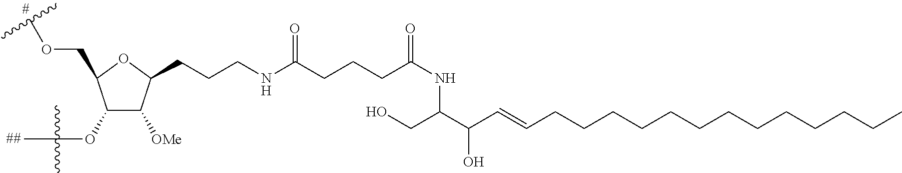 |
| Q-77 | 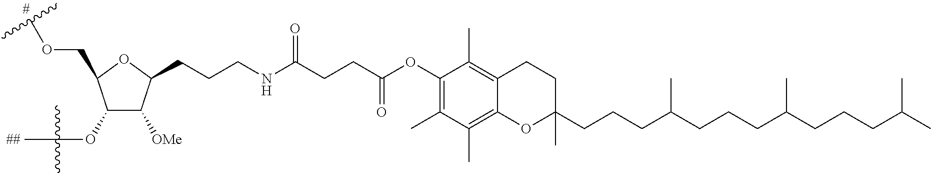 |
| Q-79 | 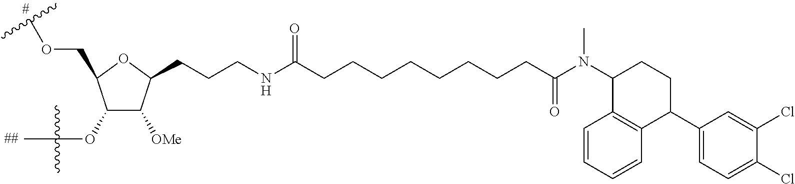 |

In some embodiments, the targeting ligand comprises a structure selected from the structures described in Table 2, wherein when the targeting ligand is at the 3′-terminus of the oligonucleotide, then # is an attachment to the rest of the conjugate; and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(—O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the targeting ligand comprises a structure selected from the structures described in Table 2, wherein when the targeting ligand is at the 3′-terminus of the oligonucleotide, then # is an attachment to the rest of the conjugate; and ## is H.

In some embodiments, the conjugate comprises a structure selected from the structures described in Table 2 at the 3′-terminus of the oligonucleotide, wherein # is an attachment to the rest of the conjugate, and ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises a structure selected from the structures described in Table 2 at the 3′-terminus of the oligonucleotide, wherein # is an attachment to the rest of the conjugate, and ## is C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano.

In some embodiments, the conjugate comprises a structure selected from the structures described in Table 2 at the 3′-terminus of the oligonucleotide, wherein # is an attachment to the rest of the conjugate, and ## is H.

In some embodiments, the targeting ligand comprises a structure selected from the structures described in Table 2, wherein the ## comprises a phosphate group (i.e., a terminal phosphate group:

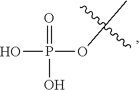 [SMILES: *OP(=O)(O)O]

or a non-terminal phosphate group:

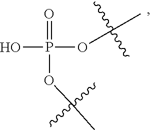 [SMILES: *OP(=O)(O)O*]

wherein each [symbol] independently is an attachment to the rest of the conjugate). In some embodiments, the phosphate group comprises a phosphodiester bond. In some embodiments, the phosphate group is a terminal group.

In some embodiments, the targeting ligand comprises a structure selected from the structures described in Table 2, wherein the ## comprises a thiophosphate group (i.e., a terminal thiophosphate group:

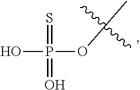

or a non-terminal thiophosphate group:

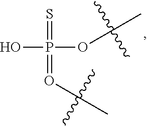

wherein each [symbol] independently is an attachment to the rest of the conjugate). In some embodiments, the thiophosphate group comprises a phosphorothioate bond. In some embodiments, the phosphorothioate group is a terminal group.

In some embodiments, the targeting ligand comprises a structure selected from Q-1, Q-3, Q-13, Q-15, Q-17, Q-19, Q-21, Q-23, Q-25, Q-27, Q-29, Q-31, Q-33, Q-35, Q-37, Q-39, Q-41, Q-43, Q-45, Q-47, Q-49, Q-65, Q-67, and Q-71 of Table 2.

In some embodiments, the targeting ligand comprises at least one phosphocholine moiety.

In some embodiments, the targeting ligand comprises:

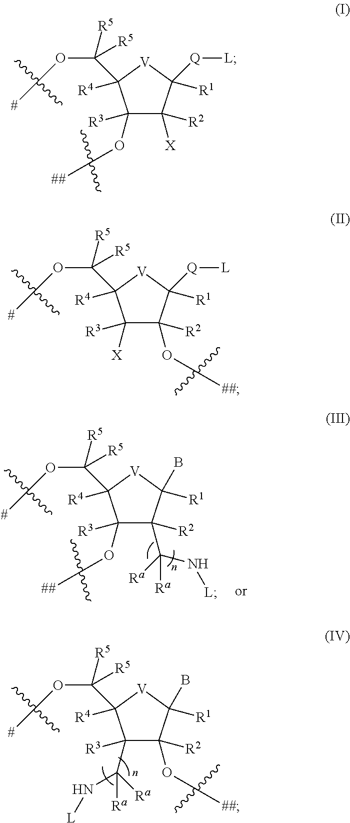

wherein:
   L comprises at least one phosphocholine moiety;
   B is H, C₁-C₆ alkyl, or a nucleobase moiety;
   V is —O—, —NR^{V}—, or —C(R^{V})₂—;
   each R^{V} independently is H or C₁-C₆ alkyl optionally substituted with one or more halogen;
   Q is —(CR^{a}R^{a})ₙ—NH—* in which * denotes attachment to L; or Q is C₆-C₁₀ arylene or 5- to 10-membered heteroarylene, wherein the C₆-C₁₀ arylene or 5- to 10-membered heteroarylene is optionally substituted with one or more R^{Q};
   each R^{Q} independently is halogen or C₁-C₆ alkyl optionally substituted with one or more halogen;
   X is H, halogen, or —OR^{X};
   R^{X} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa}; or R^{X} and R⁴ together form C₁-C₆ alkylene;
   each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
   each R^{a} independently is H, halogen, or C₁-C₆ alkyl optionally substituted with one or more halogen;
   R¹ is H, halogen, or C₁-C₆ alkyl optionally substituted with one or more halogen;
   R² is H, halogen, or C₁-C₆ alkyl optionally substituted with one or more halogen;
   R³ is H, halogen, or C₁-C₆ alkyl optionally substituted with one or more halogen;
   R⁴ is H, halogen, or C₁-C₆ alkyl optionally substituted with one or more halogen; or R⁴ and R^{X} together form C₁-C₆ alkylene;
   each R⁵ independently is H, halogen, or C₁-C₆ alkyl optionally substituted with one or more halogen; and
   n is an integer ranging from about 0 to about 10; and
when the targeting ligand is at the 3′-terminus of a Nucleic Acid Agent,
   # is an attachment to the rest of the conjugate; and
   ## is H, C₁-C₆ alkyl optionally substituted with one or more halogen, —P(R^{Y})₂, —P(OR^{Y})(N(R^{Y})₂), —P(═O)(OR^{Y})R^{Y}, —P(═S)(OR^{Y})R^{Y}, —P(═O)(SR^{Y})R^{Y}, —P(═S)(SR^{Y})R^{Y}, —P(═O)(OR^{Y})₂, —P(═S)(OR^{Y})₂, —P(═O)(SR^{Y})₂, —P(═S)(SR^{Y})₂, or a hydroxy protecting group, wherein each R^{Y} independently is H or C₁-C₆ alkyl optionally substituted with one or more halogen or cyano; or
when the targeting ligand is at the 5′-terminus of a Nucleic Acid Agent,
   # is H, —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, —P(═S)(SR^{Z})₂, or a hydroxy protecting group, wherein each R^{Z} independently is H or C₁-C₆ alkyl optionally substituted with one or more halogen or cyano; and
   ## is an attachment to the rest of the conjugate; or
   each of # and ## independently is an attachment to the rest of the conjugate.

In some embodiments, L comprises

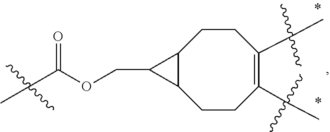 [SMILES: *C(=O)OCC1C2CC/C(*)=C(/*)CCC21]

wherein each [symbol] independently indicates an attachment to the phosphocholine moiety. In some embodiments, L comprises

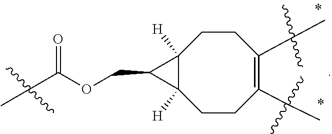 [SMILES: *C(=O)OC[C@@H]1[C@@H]2CC/C(*)=C(/*)CC[C@@H]21]

In some embodiments, the phosphocholine moiety is selected from the structures described in Table 3, or an ionized form thereof.

TABLE 3
Structures of Phosphocholine Moieties

| No. | Structure |
|---|---|
| B-1 | 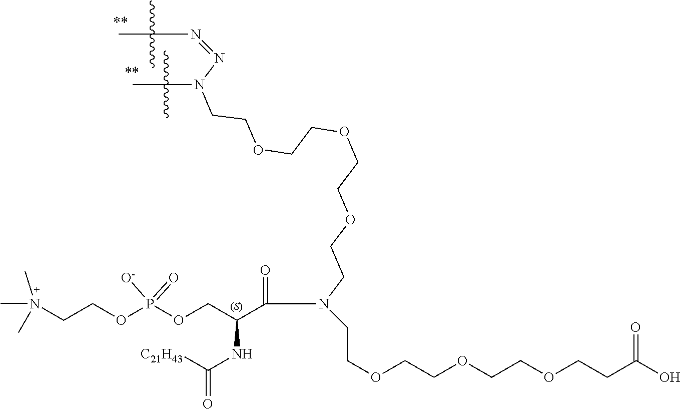 |
| B-2 | 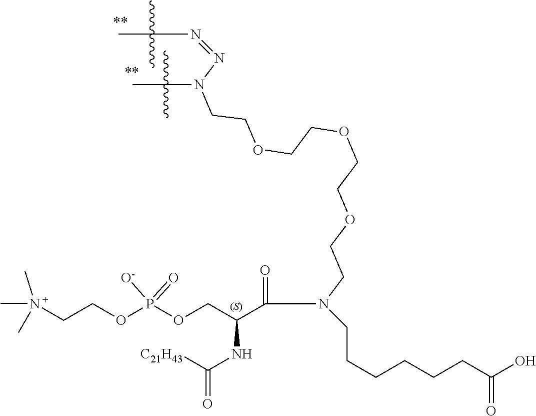 |
| B-3 | 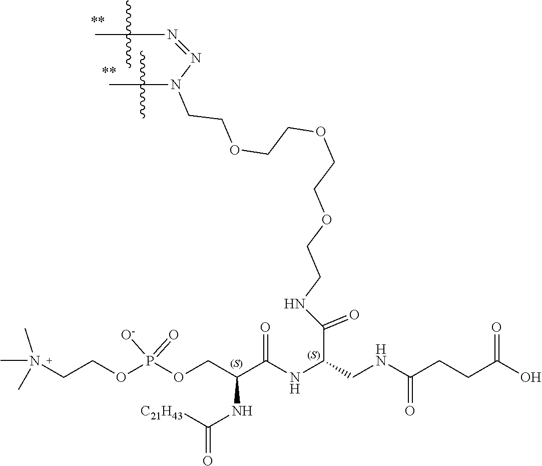 |
| B-4 | 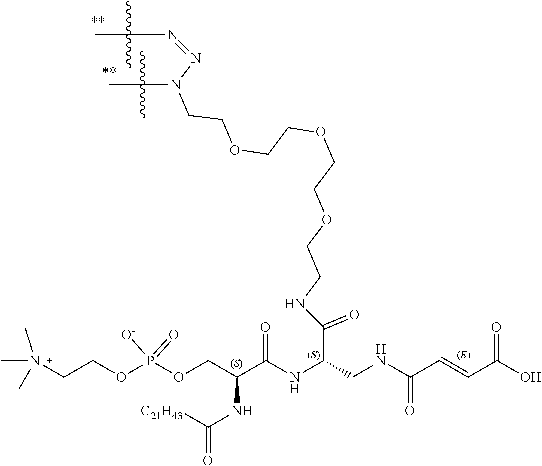 |
| B-5 | 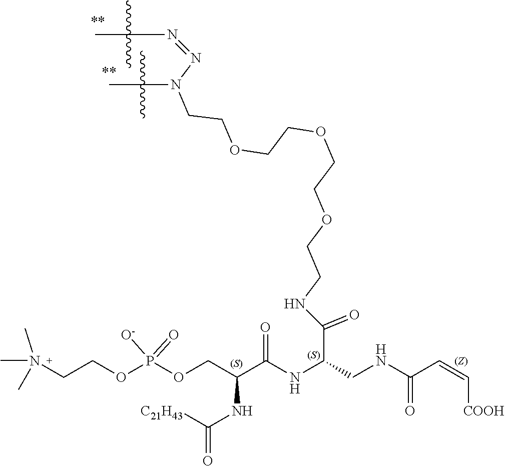 |
| B-6 | 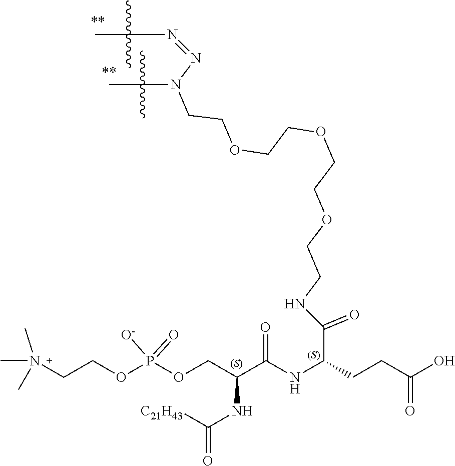 |
| B-7 | 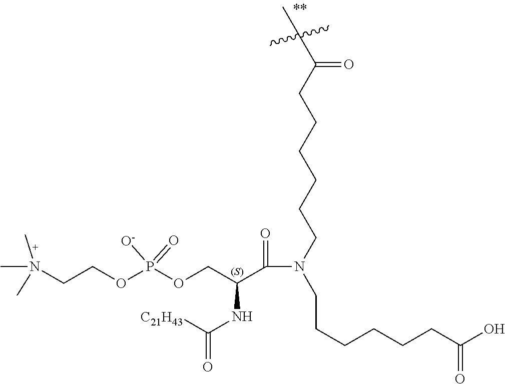 |
| B-8 | 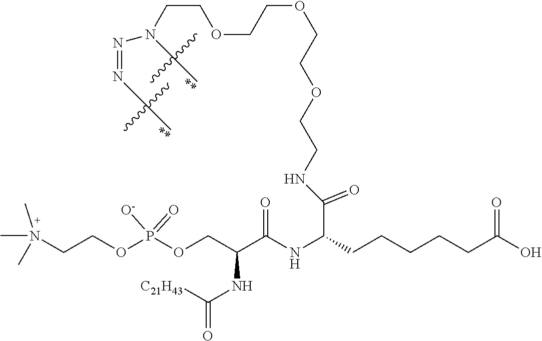 |
| B-9 | 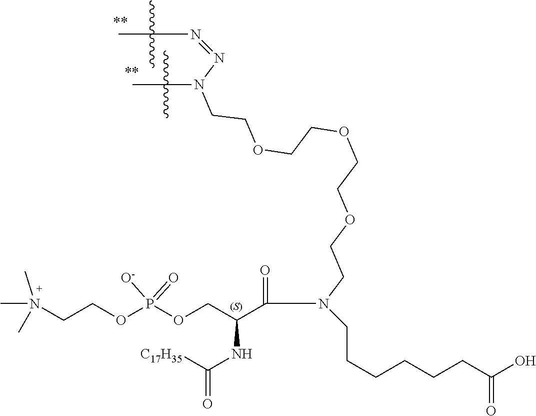 |
| B-10 | 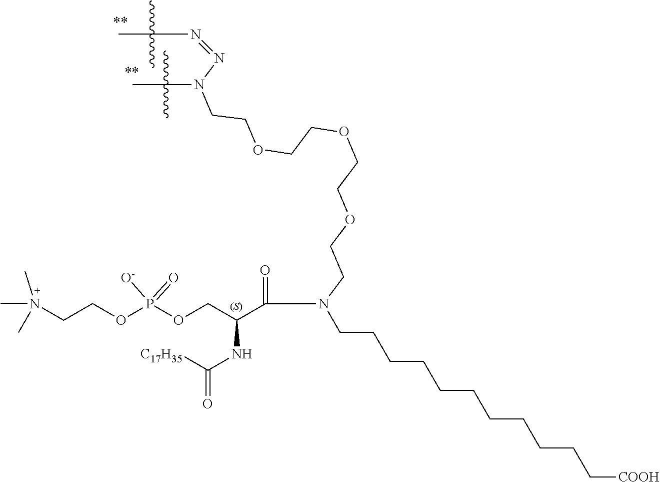 |
| B-11 | 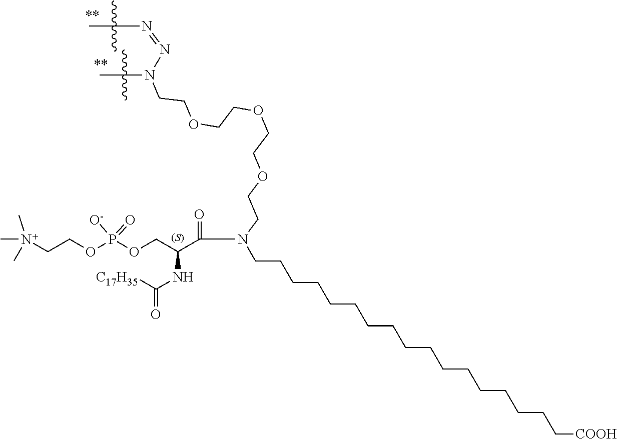 |
| B-12 | 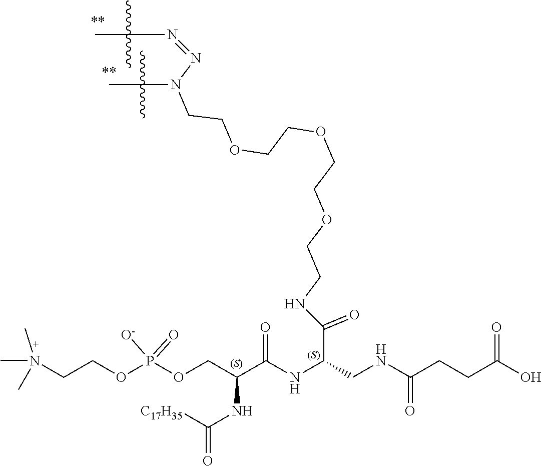 |
| B-13 | 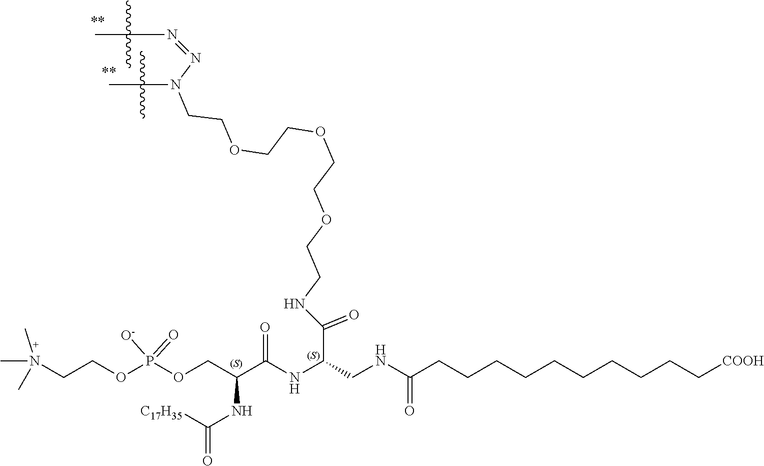 |
| B-14 | 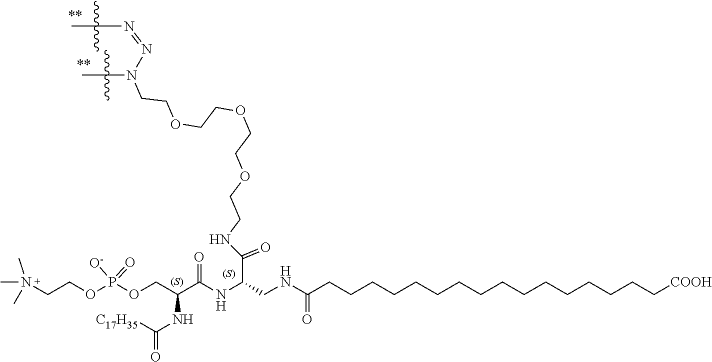 |
| B-15 | 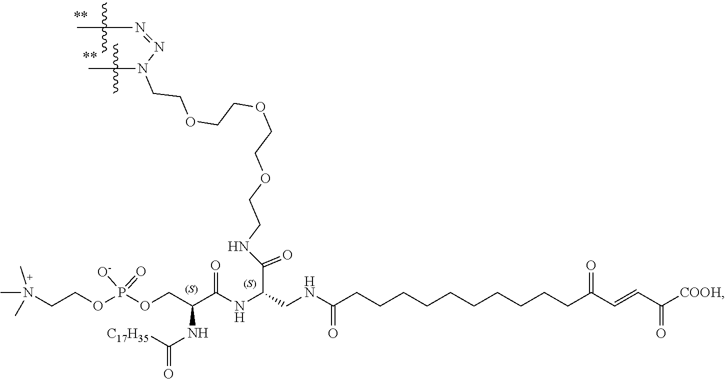 |
| B-20 | 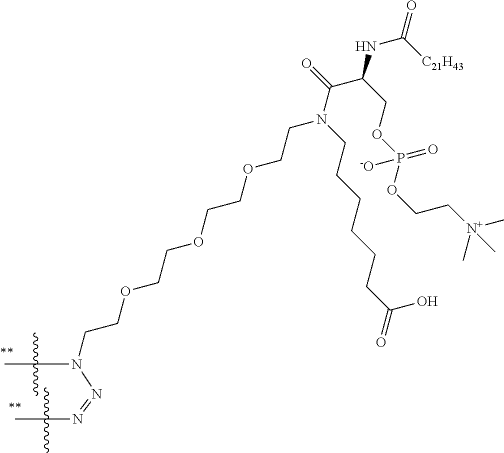 |
| B-21 | 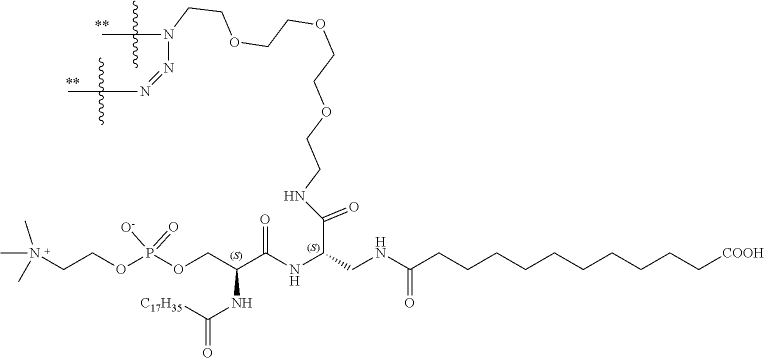 |
| B-22 | 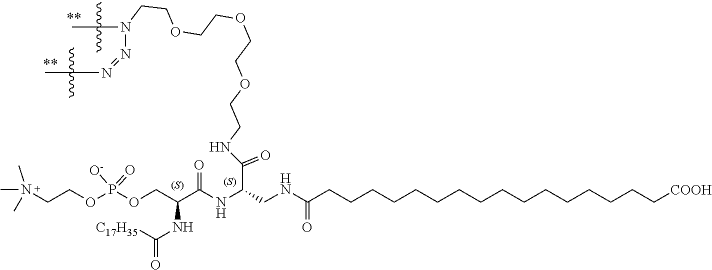 |
| B-23 | 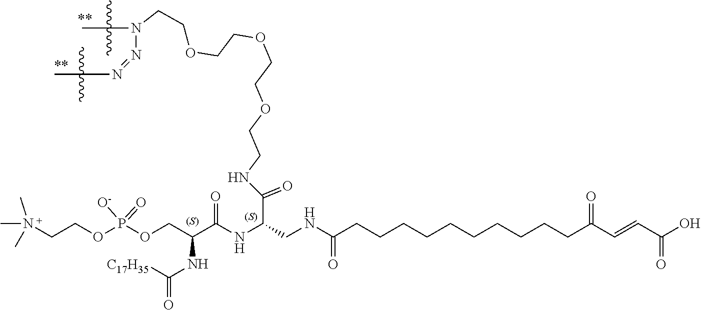 |
| B-24 | 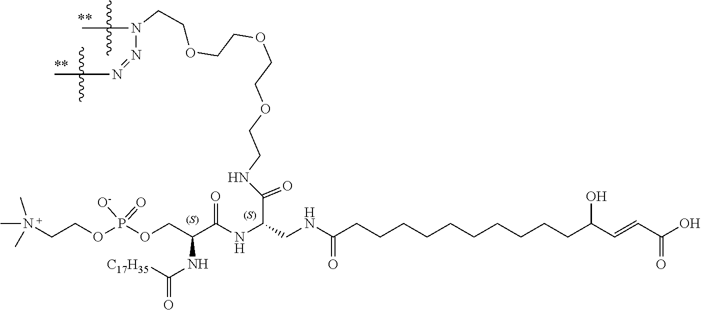 |
| B-25 | 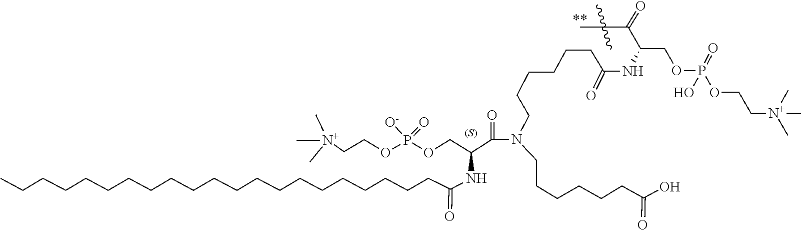 |
| B-26 | 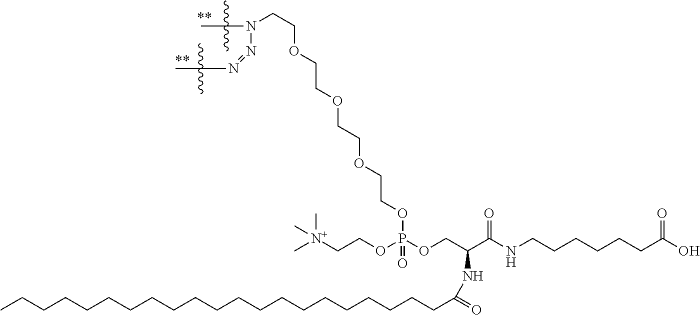 [SMILES: */N=N\N(*)CCOCCOCCOCCOP(=O)(OCC[N+](C)(C)C)OC[C@H](NC(=O)CCCCCCCCCCCCCCCCCCCCC)C(=O)NCCCCCCC(=O)O] |
| B-27 | 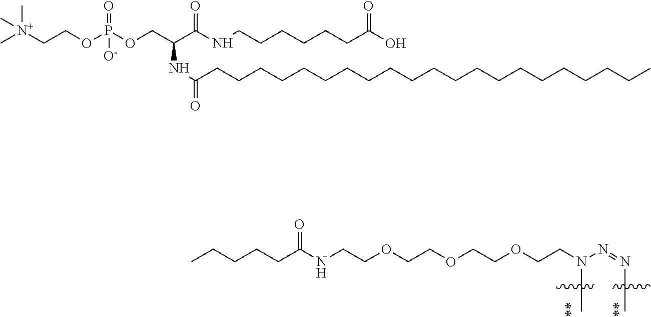 |
| B-28 | 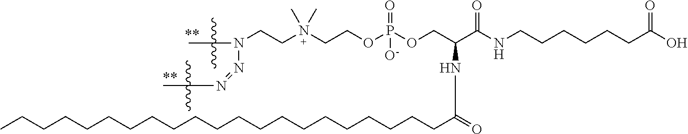 |
| B-29 | 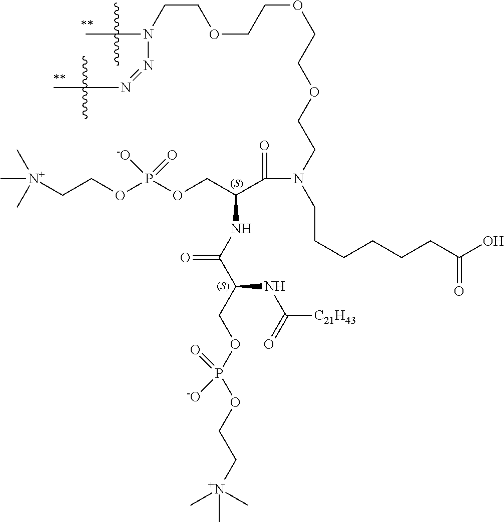 |
| B-30 | 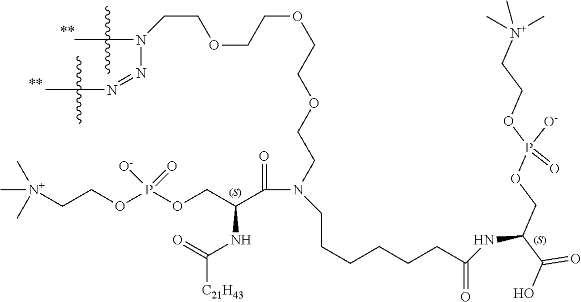 |
| B-31 | 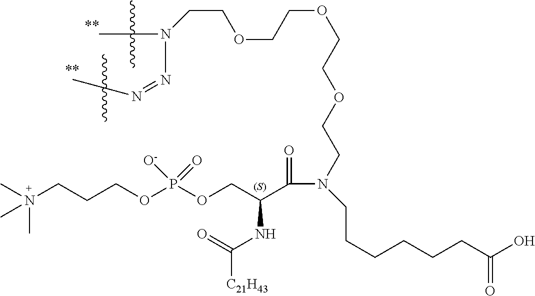 |
| B-32 | 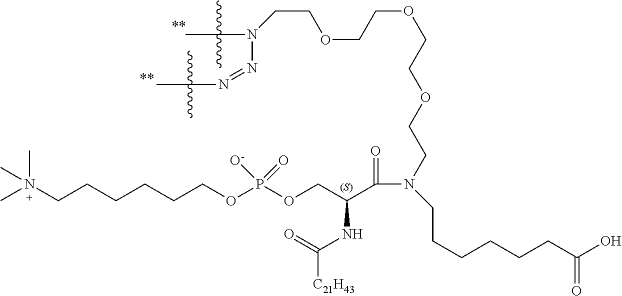 |
| B-33 | 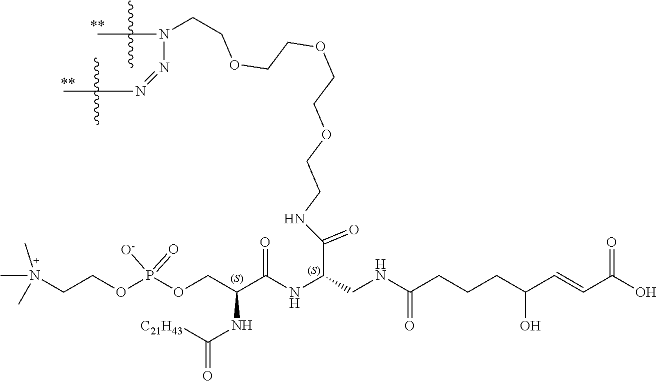 |
| B-34 | 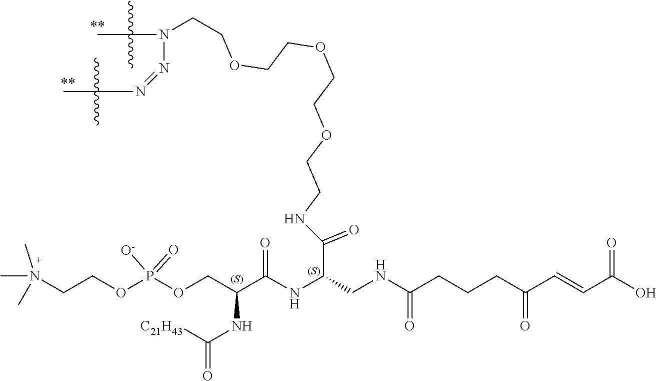 |
| B-35 | 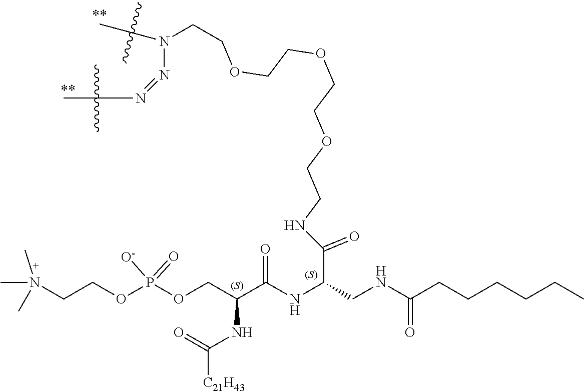 |
| B-36 | 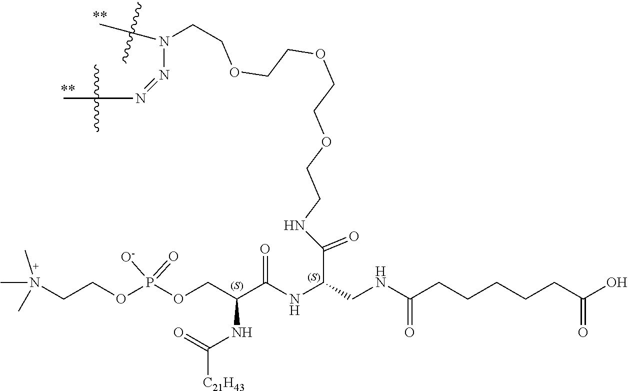 |
| B-37 | 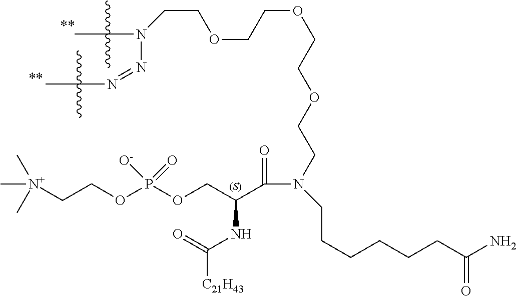 |
| B-39 | 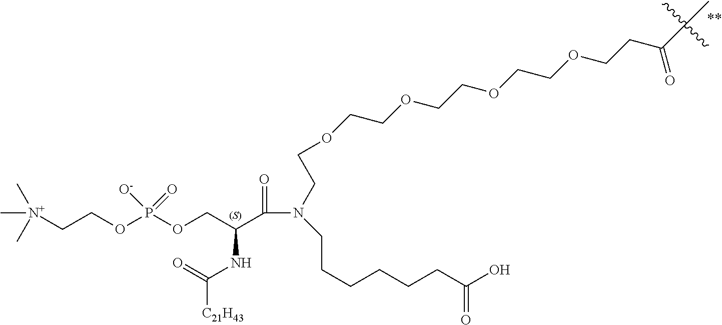 |
| B-40 | 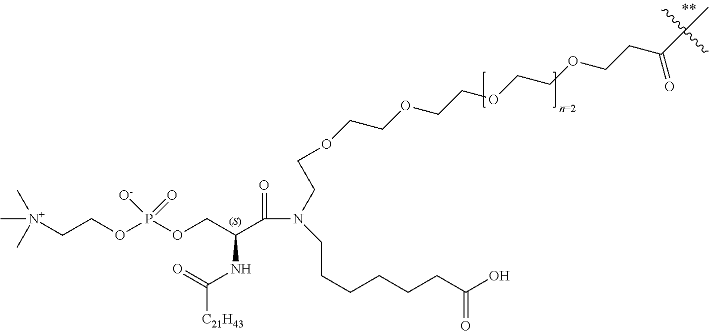 |
| B-41 | 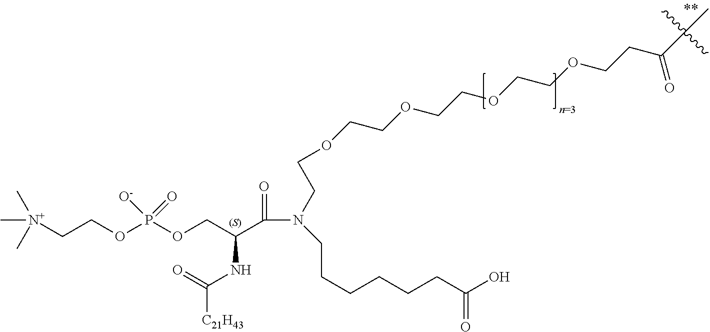 |

wherein each [symbol] independently indicates an attachment to the rest of the targeting ligand.

In some embodiments, the phosphocholine moiety is B-1, B-2, B-3, B-4, B-5, B-6, B-7, B-8, B-9, B-10, B-11, B-39, B-40, or B-41.

In some embodiments, the phosphocholine moiety is B-1, B-2, B-3, B-4, B-5, B-6, B-7, or B-8.

In some embodiments, the phosphocholine moiety is B-39, B-40, or B-41.

In some embodiments, the phosphocholine moiety is selected from the structures described in Table 3′, or an ionized form thereof.

TABLE 3′
Structures of Phosphocholine Moieties

| No. | Structure |
|---|---|
| B-16 | 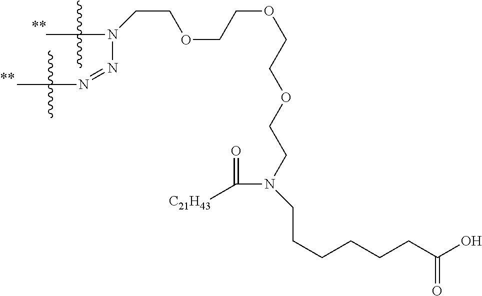 |
| B-17 | 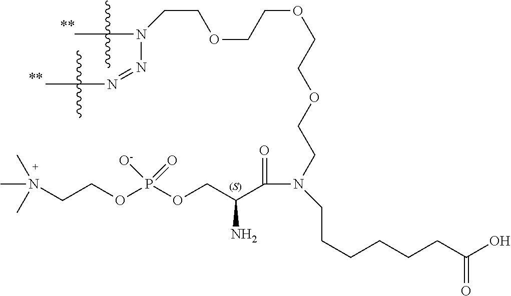 |
| B-18 | 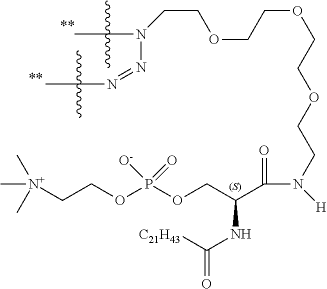 |

Suitable phosphocholine ligands include, but are not limited to, the ligands disclosed in PCT Application No. PCT/US2025/054401, which is incorporated by reference.

In some embodiments, the targeting ligand comprises a peptide or a peptide moiety (e.g., one, two, or three peptide moiety). In some embodiments, the peptide moiety comprises (e.g., one, two, or three of) integrin, insulin, glucagon-like peptide, or any combination thereof. In some embodiments, the targeting ligand comprises an antibody or an antibody moiety (e.g., transferrin). In some embodiments, the targeting ligand comprises one, two, or three antibody moieties (e.g., transferrin).

In some embodiments, the targeting ligand comprises an oligonucleotide (e.g., aptamer or CpG). In some embodiments, the targeting ligand comprises one, two, or three oligonucleotides (e.g., aptamer or CpG).

In some embodiments, the ligand comprises: one, two, or three sugar (e.g., N-acetyl-D-galactosylamine); one, two, or three lipid moieties; one, two, or three peptide moieties; one, two, or three antibody moieties; one, two, or three oligonucleotides; or any combination thereof.

In some embodiments, the linker is attached to the isolated oligonucleotide of the present disclosure, via a phosphate group, or an analog of a phosphate group, in the isolated oligonucleotide.

In some embodiments of the isolated oligonucleotide of the present disclosure, the isolated oligonucleotide is linked to the ligand. In some embodiments, the isolated oligonucleotide is linked to the ligand via an internal or terminal nucleotide of the isolated oligonucleotide. In some embodiments, the isolated oligonucleotide is linked to the ligand via a ligand linker.

In some embodiments of the isolated oligonucleotide of the present disclosure, wherein the isolated oligonucleotide comprises a sense and an antisense strand, and wherein the ligand located on the 3′ end of the sense strand, the ligand is linked to a terminal nucleotide on the sense strand of the isolated oligonucleotide. In some embodiments, the ligand is linked to a terminal nucleotide on the sense strand via a ligand linker. In some embodiments, the ligand linker is a monovalent linker. In some embodiments, the ligand linker is a bivalent linker. In some embodiments, the ligand linker is a trivalent linker.

In some embodiments, the ligand comprises a sugar ligand moiety (e.g., N-acetylgalactosamine (GalNAc)) which may direct uptake of an oligonucleotide into the liver.

In some embodiments, the ligand comprises GalNAc, or a derivative thereof. In some embodiments, the ligand comprises a GalNAc G1b structure shown below:

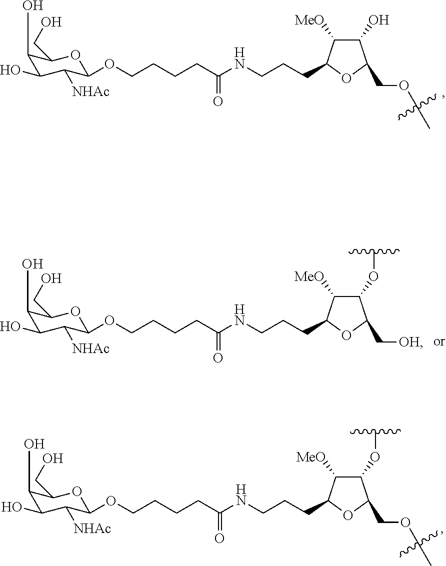 [SMILES: *OC[C@H]1O[C@@H](CCCNC(=O)CCCCOC2OC(CO)C(O)C(O)C2NC(C)=O)[C@H](OC)[C@@H]1O ; *O[C@H]1[C@@H](OC)[C@H](CCCNC(=O)CCCCOC2OC(CO)C(O)C(O)C2NC(C)=O)O[C@@H]1CO ; *OC[C@H]1O[C@@H](CCCNC(=O)CCCCOC2OC(CO)C(O)C(O)C2NC(C)=O)[C@H](OC)[C@@H]1O*]

wherein each [symbol] independently indicates the point of attachment to the oligonucleotide compound or the other G1b moiety,
   GalNAc G1b (G1b or [G1b])

In some embodiments, the ligand comprises three GalNAc moieties, or three derivatives thereof. In some embodiments, the ligand comprises three GalNAc G1b moieties. In some embodiments, wherein the ligand comprises three GalNAc G1b moieties, the GalNAc G1b moieties are consecutively located. In some embodiments, the consecutively located GalNAc G1b moieties are located on the 3′ end of the sense strand. In some embodiments, wherein the ligand comprises three GalNAc G1b (“G1b”) moieties that are consecutively located, the first G1b moiety is linked to the second G1b moiety and the second G1b is linked to the third G1b moiety. In some embodiments, the first GalNAc G1b moiety is linked to the sense strand of the oligonucleotide compound of the present disclosure.

In some embodiments, the ligand comprises a [G1b][G1b][G1b] moiety, with structure shown below:

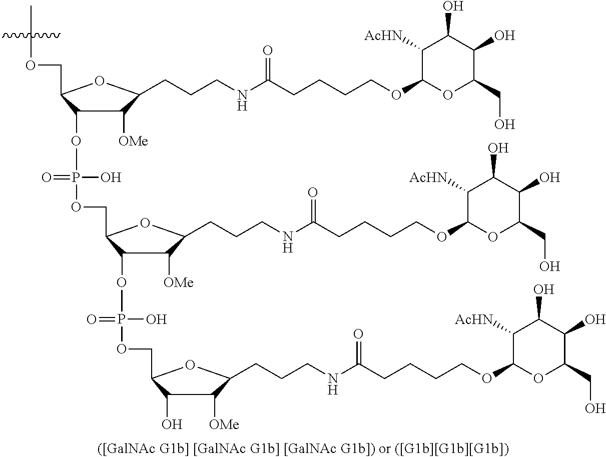

In some embodiments of the isolated oligonucleotide of the present disclosure, wherein the ligand comprises three GalNAc G1b (“G1b”) moieties, wherein the first GalNAc G1b moiety is linked to the sense strand of the isolated oligonucleotide, the first GalNAc G1b moiety is also linked to the second GalNAc G1b moiety, and the second G1b is linked to the third G1b moiety. In some embodiments, wherein the ligand comprises three GalNAc G1b moieties, the three GalNAc G1b moieties are consecutively located on the 3′ end of the sense strand.

In some embodiments of the isolated oligonucleotide of the present disclosure, the isolated oligonucleotide is linked to the ligand (e.g., GalNAc G1b, or three GalNAc G1b moieties). In some embodiments, the isolated oligonucleotide is linked to the ligand via an internal or terminal nucleotide of the isolated oligonucleotide. In some embodiments, the isolated oligonucleotide is linked to the ligand via a ligand linker. In some embodiments, the

In some embodiments of the isolated oligonucleotide of the present disclosure, wherein the isolated oligonucleotide comprises a sense strand and an antisense strand, and wherein the ligand comprises three GalNAc G1b moieties, and the three GalNAc G1b moieties are consecutively located on the 3′ end of the sense strand, the ligand is linked to a terminal nucleotide on the sense strand of the isolated oligonucleotide. In some embodiments, the ligand is linked to a terminal nucleotide on the sense strand via a ligand linker. In some embodiments, the ligand linker is a monovalent linker. In some embodiments, the ligand linker is a bivalent linker. In some embodiments, the ligand linker is a trivalent linker.

In certain embodiments, the ligand targets delivery of the RNAi construct to hepatocytes. In these and other embodiments, the ligand may comprise galactose, galactosamine or N-acetyl-galactosamine (GalNAc). In certain embodiments, the ligand comprises a multivalent galactose or multivalent GalNAc moiety, such as a trivalent or tetravalent galactose or GalNAc moiety. The ligand can be covalently attached to the 5′ or 3′ end of the sense strand of the RNAi construct, optionally via a linker.

Suitable ligands include, but are not limited to, the ligands disclosed in PCT Application No. PCT/US2022/039517 (PCT Publication No. WO 2023/014938), U.S. application Ser. No. 17/881,935 (US Publication No. US 2023-0138928), each of which is incorporated by reference.

The linkage at the 3′ end of the isolated oligonucleotide of the present disclosure may be directly via 5′, 3′ or 2′ hydroxyl groups, or indirectly, via a non-nucleotide linker or a nucleoside, utilizing either the 2′ or 3′ hydroxyl positions of the nucleoside. Linkages may also utilize a functionalized sugar or nucleobase of a 3′ terminal nucleotide. In some embodiments, the ligand described herein can be attached to the isolated oligonucleotide of the present disclosure with various ligand linkers that can be cleavable or non-cleavable.

The present disclosure provides an isolated oligonucleotide comprising a sense strand and an antisense strand, wherein the sense strand or the antisense strand or both comprise one or more modified nucleotide(s), and wherein the sense strand or the antisense strand or both comprise a terminal or internal nucleotide linked to a targeting ligand. In some embodiments, the targeting ligand comprises at least one GalNAc G1b moiety, optionally three G1b moieties. In some embodiments, the targeting ligand comprises G1b-BCN—C22PC2.

In some embodiments, the isolated oligonucleotide comprises an antisense strand comprising SEQ ID NO: 1074 and its corresponding sense strand comprising SEQ ID NO: 1079, wherein the targeting ligand comprises at least one GalNAc G1b moiety, optionally three G1b moieties. In some embodiments, the isolated oligonucleotide comprises an antisense strand comprising SEQ ID NO: 1074 and its corresponding sense strand comprising SEQ ID NO: 1079, wherein the targeting ligand comprises G1b-BCN—C22PC2.

In some embodiments, the isolated oligonucleotide comprises an antisense strand comprising SEQ ID NO: 1075 and its corresponding sense strand comprising SEQ ID NO: 1080, wherein the targeting ligand comprises at least one GalNAc G1b moiety, optionally three G1b moieties. In some embodiments, the isolated oligonucleotide comprises an antisense strand comprising SEQ ID NO: 1075 and its corresponding sense strand comprising SEQ ID NO: 1080, wherein the targeting ligand comprises G1b-BCN—C22PC2.

In some embodiments, the isolated oligonucleotide comprises an antisense strand comprising SEQ ID NO: 1076 and its corresponding sense strand comprising SEQ ID NO: 1081, wherein the targeting ligand comprises at least one GalNAc G1b moiety, optionally three G1b moieties. In some embodiments, the isolated oligonucleotide comprises an antisense strand comprising SEQ ID NO: 1076 and its corresponding sense strand comprising SEQ ID NO: 1081, wherein the targeting ligand comprises G1b-BCN—C22PC2.

In some embodiments, the isolated oligonucleotide comprises an antisense strand comprising SEQ ID NO: 1077 and its corresponding sense strand comprising SEQ ID NO: 1082, wherein the targeting ligand comprises at least one GalNAc G1b moiety, optionally three G1b moieties. In some embodiments, the isolated oligonucleotide comprises an antisense strand comprising SEQ ID NO: 1077 and its corresponding sense strand comprising SEQ ID NO: 1082, wherein the targeting ligand comprises G1b-BCN—C22PC2.

In some embodiments, the isolated oligonucleotide comprises an antisense strand comprising SEQ ID NO: 1078 and its corresponding sense strand comprising SEQ ID NO: 1083, wherein the targeting ligand comprises at least one GalNAc G1b moiety, optionally three G1b moieties. In some embodiments, the isolated oligonucleotide comprises an antisense strand comprising SEQ ID NO: 1078 and its corresponding sense strand comprising SEQ ID NO: 1083, wherein the targeting ligand comprises G1b-BCN—C22PC2.

In some embodiments, the isolated oligonucleotide comprises an antisense strand comprising SEQ ID NO: 1077 and its corresponding sense strand comprising SEQ ID NO: 1084, wherein the targeting ligand comprises at least one GalNAc G1b moiety, optionally three G1b moieties. In some embodiments, the isolated oligonucleotide comprises an antisense strand comprising SEQ ID NO: 1077 and its corresponding sense strand comprising SEQ ID NO: 1084, wherein the targeting ligand comprises G1b-BCN—C22PC2.

### Modification of the Phosphate Groups

### Modified Terminal Phosphate Groups

The present disclosure further provides oligonucleotides and conjugates containing modified phosphate groups (also referred to as phosphate mimics or phosphate derivatives) for nucleic acid delivery. The present disclosure also relates to uses of oligonucleotides and conjugates containing modified phosphate groups, e.g., in delivering nucleic acid and/or treating or preventing diseases.

In some embodiments, the present disclosure provides phosphate mimics of 5′-terminal nucleotides. Without wishing to be bound by theory, it is understood that, when being incorporated into oligonucleotides (e.g., at the 5′-terminus of the antisense strand), the phosphate mimics could improve the Ago2 binding/loading and enhance the metabolic stability of the oligonucleotides, thus enhancing the potency and duration of the isolated oligonucleotides (e.g., dsRNA or siRNA).

In some embodiments of the isolated oligonucleotides of the present disclosure, the oligonucleotides comprise 5′-terminal nucleotide modifications. In some embodiments, the 5′-terminal modifications provide the functional effect of a phosphate group, but are more stable in the environmental conditions that the oligonucleotide will be exposed to when administered to a subject. In some embodiments, the isolated oligonucleotide comprises phosphate mimics that are more resistant to phosphatases and other enzymes while minimizing negative impact on the oligonucleotide's function (e.g., minimizing any reduction in gene target knockdown when used as an RNAi inhibitor molecule).

In some embodiments, the 5′-terminal modification is a chemical modification. In some embodiments, the chemical modification enhances stability against nucleases or other enzymes that degrade or interfere with the structure or activity of the isolated oligonucleotide.

In some embodiments, the sense or antisense strand of the isolated oligonucleotides of the present disclosure comprise a 5′-terminal phosphate group. In some embodiments, the 5′-terminal phosphate group comprises an unmodified phosphate having the formula: —O—P(═O)(OH)OH. In some embodiments, the 5′-terminal phosphate group comprises a modified phosphate. In some embodiments, the 5′-terminal phosphate group comprises a modified phosphate having the formula —CH₂—P(═X)(OR¹)OR², wherein X is O or S, R¹ is H or C₁-C₆ alkyl, and R² is H or C₁-C₆ alkyl. In some embodiments, the modified phosphate is referred to as a “phosphate mimic”.

In some embodiments, the sense or antisense strand of the isolated oligonucleotides of the present disclosure comprises a 5′-terminal phosphate mimic.

In some embodiments, the phosphate mimic is linked to the 5′-terminus of the isolated oligonucleotides (e.g., siRNAs) as shown in the following formula:

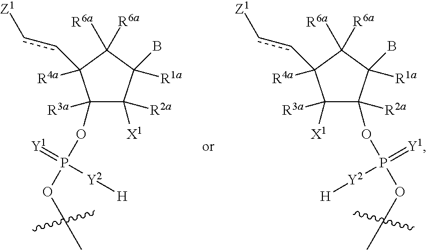

wherein:
   B is H or a nucleobase moiety;
   X¹ is H, halogen, or —OR^{X};
   R^{X} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Xa};
   each R^{Xa} independently is halogen, C₁-C₆ alkyl, or —O—(C₁-C₆ alkyl), wherein the C₁-C₆ alkyl or —O—(C₁-C₆ alkyl) is optionally substituted with one or more halogen;
   Y¹ is O or S;
   Y² is O or S;
   Z¹ is —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, or —P(═S)(SR^{Z})₂;
   each R^{Z} independently is H or C₁-C₆ alkyl optionally substituted with one or more halogen or cyano;
   R^{1a} is H, halogen, or C₁-C₆ alkyl optionally substituted with one or more halogen;
   R^{2a} is H, halogen, or C₁-C₆ alkyl optionally substituted with one or more halogen;
   R^{3a} is H, halogen, or C₁-C₆ alkyl optionally substituted with one or more halogen;
   R^{4a} is H, halogen, or C₁-C₆ alkyl optionally substituted with one or more halogen;
   [symbol] indicates a single bond or a double bond;
   each R^{6a} independently is H, halogen, or C₁-C₆ alkyl optionally substituted with one or more halogen; and
   [symbol] indicates an attachment to the rest of the isolated oligonucleotide (e.g., siRNA).

In some embodiments, the phosphate mimic is linked to the 5′-terminus of the isolated oligonucleotides (e.g., siRNAs) as shown in the following formula:

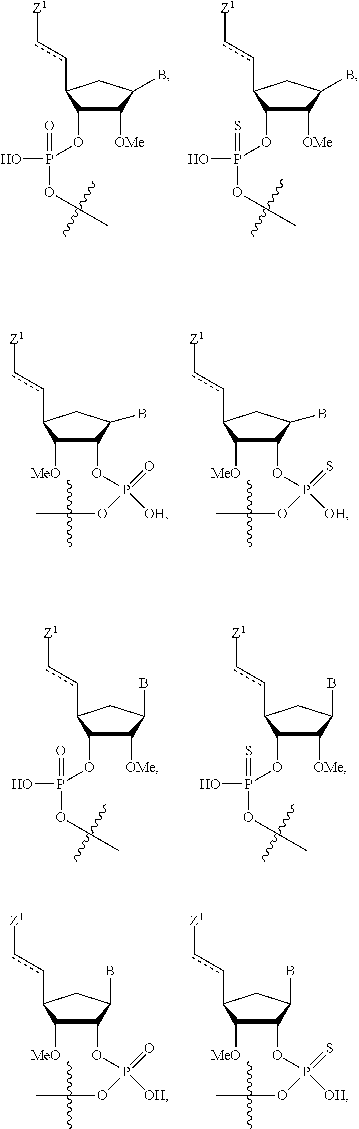

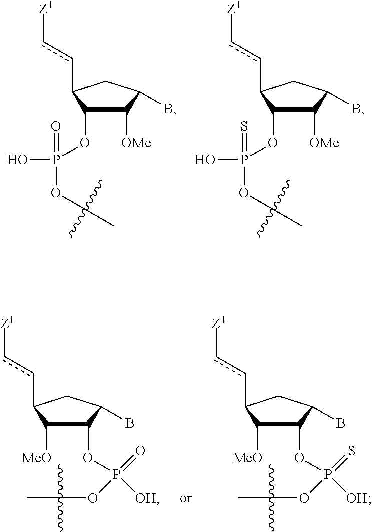

wherein:
   Z¹ is —P(R^{Z})₂, —P(OR^{Z})(N(R^{Z})₂), —P(═O)(OR^{Z})R^{Z}, —P(═S)(OR^{Z})R^{Z}, —P(═O)(SR^{Z})R^{Z}, —P(═S)(SR^{Z})R^{Z}, —P(═O)(OR^{Z})₂, —P(═S)(OR^{Z})₂, —P(═O)(SR^{Z})₂, or —P(═S)(SR^{Z})₂;
   each R^{Z} independently is H or C₁-C₆ alkyl optionally substituted with one or more halogen or cyano.

In some embodiments, the phosphate mimic is linked to the 5′-terminus of the isolated oligonucleotides (e.g., siRNAs) as shown in the following structures of Table 4:

TABLE 4
Phosphate Mimic Structure

| Compound No. | Structure |
|---|---|
| M-1 | 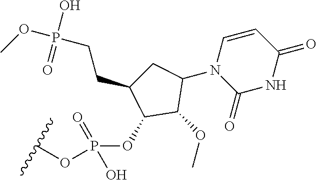 [SMILES: *OP(=O)(O)O[C@@H]1[C@@H](CCP(=O)(O)OC)CC(n2ccc(=O)[nH]c2=O)[C@@H]1OC] |
| M-2 | 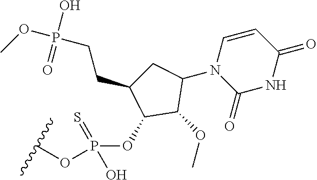 [SMILES: *OP(O)(=S)O[C@@H]1[C@@H](CCP(=O)(O)OC)CC(n2ccc(=O)[nH]c2=O)[C@@H]1OC] |
| M-3 | 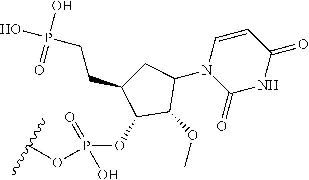 [SMILES: *OP(=O)(O)O[C@@H]1[C@@H](CCP(=O)(O)O)CC(n2ccc(=O)[nH]c2=O)[C@@H]1OC] |
| M-4 | 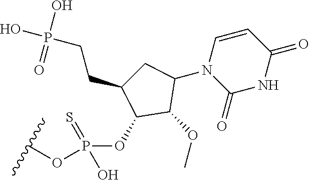 [SMILES: *OP(O)(=S)O[C@@H]1[C@@H](CCP(=O)(O)O)CC(n2ccc(=O)[nH]c2=O)[C@@H]1OC] |
| M-5 | 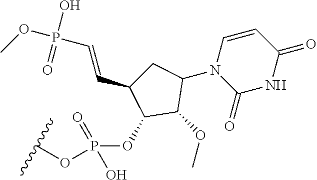 [SMILES: *OP(=O)(O)O[C@@H]1[C@@H](/C=C/P(=O)(O)OC)CC(n2ccc(=O)[nH]c2=O)[C@@H]1OC] |
| M-6 | 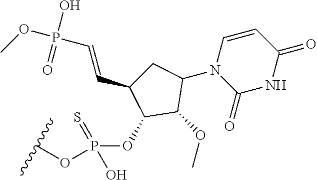 [SMILES: *OP(O)(=S)O[C@@H]1[C@@H](/C=C/P(=O)(O)OC)CC(n2ccc(=O)[nH]c2=O)[C@@H]1OC] |
| M-7 | 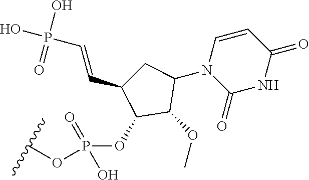 [SMILES: *OP(=O)(O)O[C@@H]1[C@@H](/C=C/P(=O)(O)O)CC(n2ccc(=O)[nH]c2=O)[C@@H]1OC] |
| M-8 | 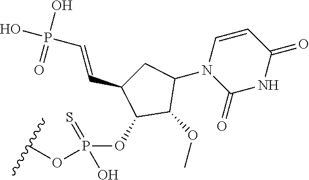 [SMILES: *OP(O)(=S)O[C@@H]1[C@@H](/C=C/P(=O)(O)O)CC(n2ccc(=O)[nH]c2=O)[C@@H]1OC] |
| M-9 | 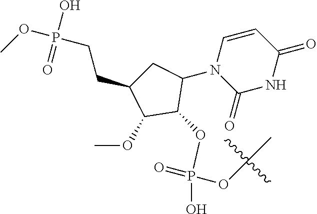 [SMILES: *OP(=O)(O)O[C@H]1C(n2ccc(=O)[nH]c2=O)C[C@H](CCP(=O)(O)OC)[C@H]1OC] |
| M-10 | 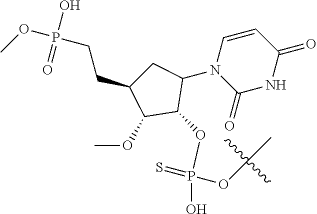 [SMILES: *OP(O)(=S)O[C@H]1C(n2ccc(=O)[nH]c2=O)C[C@H](CCP(=O)(O)OC)[C@H]1OC] |
| M-11 | 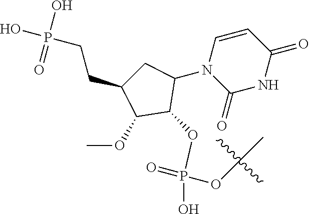 [SMILES: *OP(=O)(O)O[C@H]1C(n2ccc(=O)[nH]c2=O)C[C@H](CCP(=O)(O)O)[C@H]1OC] |
| M-12 | 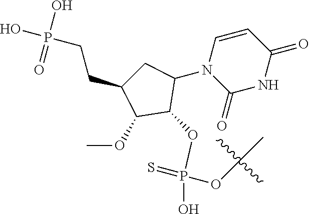 [SMILES: *OP(O)(=S)O[C@H]1C(n2ccc(=O)[nH]c2=O)C[C@H](CCP(=O)(O)O)[C@H]1OC] |
| M-13 | 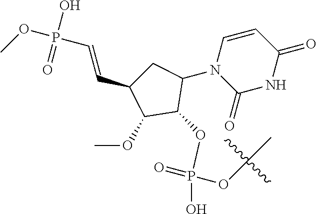 [SMILES: *OP(=O)(O)O[C@H]1C(n2ccc(=O)[nH]c2=O)C[C@H](/C=C/P(=O)(O)OC)[C@H]1OC] |
| M-14 | 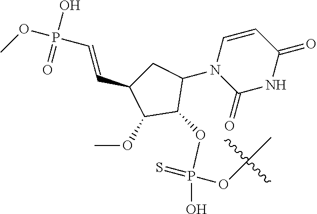 [SMILES: *OP(O)(=S)O[C@H]1C(n2ccc(=O)[nH]c2=O)C[C@H](/C=C/P(=O)(O)OC)[C@H]1OC] |
| M-15 | 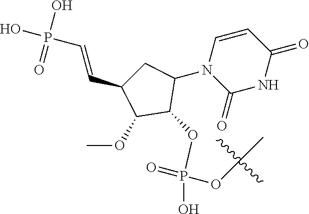 [SMILES: *OP(=O)(O)O[C@H]1C(n2ccc(=O)[nH]c2=O)C[C@H](/C=C/P(=O)(O)O)[C@H]1OC] |
| M-16 | 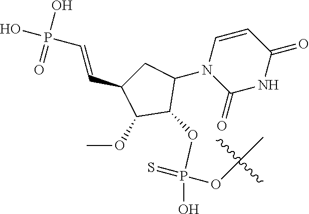 [SMILES: *OP(O)(=S)O[C@H]1C(n2ccc(=O)[nH]c2=O)C[C@H](/C=C/P(=O)(O)O)[C@H]1OC] |
| M-17 | 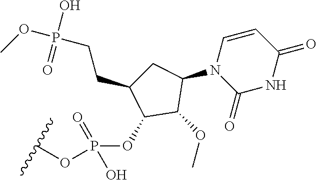 [SMILES: *OP(=O)(O)O[C@@H]1[C@@H](CCP(=O)(O)OC)C[C@@H](n2ccc(=O)[nH]c2=O)[C@@H]1OC] |
| M-18 | 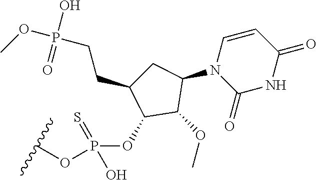 [SMILES: *OP(O)(=S)O[C@@H]1[C@@H](CCP(=O)(O)OC)C[C@@H](n2ccc(=O)[nH]c2=O)[C@@H]1OC] |
| M-19 | 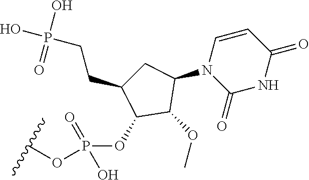 [SMILES: *OP(=O)(O)O[C@@H]1[C@@H](CCP(=O)(O)O)C[C@@H](n2ccc(=O)[nH]c2=O)[C@@H]1OC] |
| M-20 | 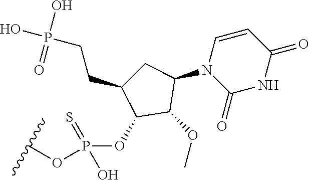 [SMILES: *OP(O)(=S)O[C@@H]1[C@@H](CCP(=O)(O)O)C[C@@H](n2ccc(=O)[nH]c2=O)[C@@H]1OC] |
| M-21 | 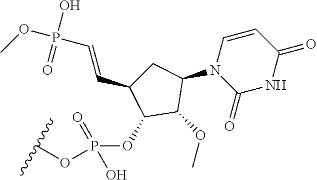 [SMILES: *OP(=O)(O)O[C@H]1[C@@H](OC)[C@H](n2ccc(=O)[nH]c2=O)C[C@@H]1/C=C/P(=O)(O)OC] |
| M-22 | 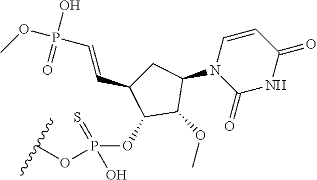 [SMILES: *OP(O)(=S)O[C@H]1[C@@H](OC)[C@H](n2ccc(=O)[nH]c2=O)C[C@@H]1/C=C/P(=O)(O)OC] |
| M-23 | 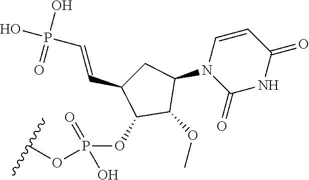 [SMILES: *OP(=O)(O)O[C@H]1[C@@H](OC)[C@H](n2ccc(=O)[nH]c2=O)C[C@@H]1/C=C/P(=O)(O)O] |
| M-24 | 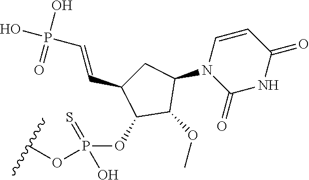 [SMILES: *OP(O)(=S)O[C@H]1[C@@H](OC)[C@H](n2ccc(=O)[nH]c2=O)C[C@@H]1/C=C/P(=O)(O)O] |
| M-25 | 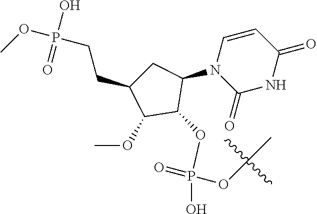 [SMILES: *OP(=O)(O)O[C@@H]1[C@H](OC)[C@@H](CCP(=O)(O)OC)C[C@H]1n1ccc(=O)[nH]c1=O] |
| M-26 | 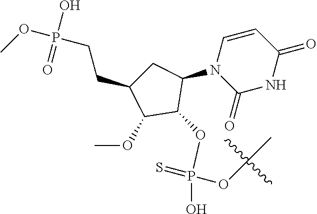 [SMILES: *OP(O)(=S)O[C@@H]1[C@H](OC)[C@@H](CCP(=O)(O)OC)C[C@H]1n1ccc(=O)[nH]c1=O] |
| M-27 | 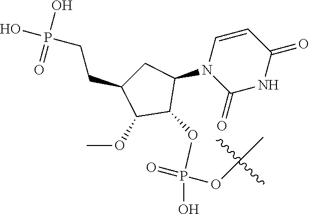 [SMILES: *OP(=O)(O)O[C@@H]1[C@H](OC)[C@@H](CCP(=O)(O)O)C[C@H]1n1ccc(=O)[nH]c1=O] |
| M-28 | 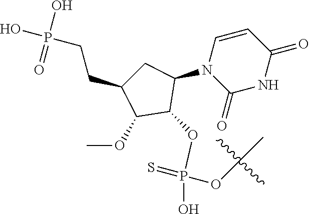 [SMILES: *OP(O)(=S)O[C@@H]1[C@H](OC)[C@@H](CCP(=O)(O)O)C[C@H]1n1ccc(=O)[nH]c1=O] |
| M-29 | 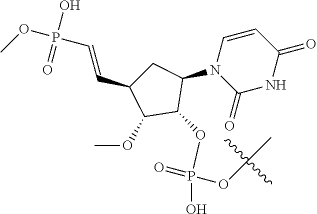 [SMILES: *OP(=O)(O)O[C@@H]1[C@H](OC)[C@@H](/C=C/P(=O)(O)OC)C[C@H]1n1ccc(=O)[nH]c1=O] |
| M-30 | 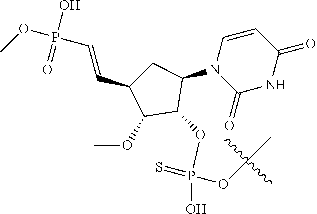 [SMILES: *OP(O)(=S)O[C@@H]1[C@H](OC)[C@@H](/C=C/P(=O)(O)OC)C[C@H]1n1ccc(=O)[nH]c1=O] |
| M-31 | 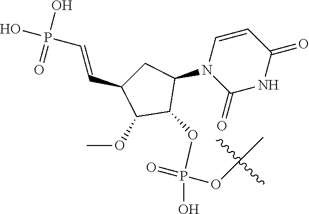 [SMILES: *OP(=O)(O)O[C@@H]1[C@H](OC)[C@@H](/C=C/P(=O)(O)O)C[C@H]1n1ccc(=O)[nH]c1=O] |
| M-32 | 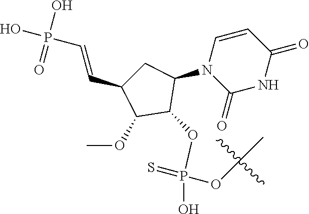 [SMILES: *OP(O)(=S)O[C@@H]1[C@H](OC)[C@@H](/C=C/P(=O)(O)O)C[C@H]1n1ccc(=O)[nH]c1=O] |
| M-33 | 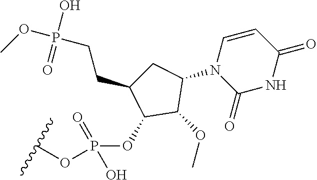 [SMILES: *OP(=O)(O)O[C@@H]1[C@@H](CCP(=O)(O)OC)C[C@H](n2ccc(=O)[nH]c2=O)[C@@H]1OC] |
| M-34 | 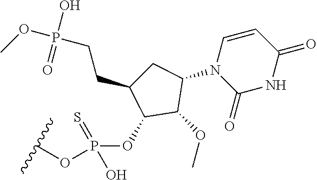 [SMILES: *OP(O)(=S)O[C@@H]1[C@@H](CCP(=O)(O)OC)C[C@H](n2ccc(=O)[nH]c2=O)[C@@H]1OC] |
| M-35 | 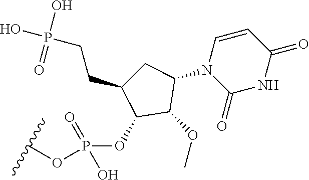 [SMILES: *OP(=O)(O)O[C@@H]1[C@@H](CCP(=O)(O)O)C[C@H](n2ccc(=O)[nH]c2=O)[C@@H]1OC] |
| M-36 | 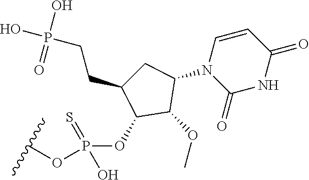 [SMILES: *OP(O)(=S)O[C@@H]1[C@@H](CCP(=O)(O)O)C[C@H](n2ccc(=O)[nH]c2=O)[C@@H]1OC] |
| M-37 | 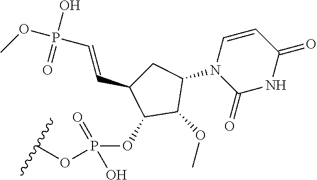 [SMILES: *OP(=O)(O)O[C@H]1[C@@H](OC)[C@@H](n2ccc(=O)[nH]c2=O)C[C@@H]1/C=C/P(=O)(O)OC] |
| M-38 | 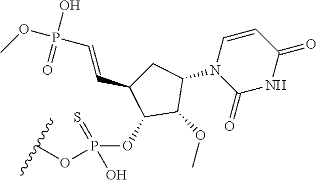 [SMILES: *OP(O)(=S)O[C@H]1[C@@H](OC)[C@@H](n2ccc(=O)[nH]c2=O)C[C@@H]1/C=C/P(=O)(O)OC] |
| M-39 | 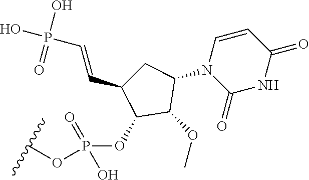 [SMILES: *OP(=O)(O)O[C@H]1[C@@H](OC)[C@@H](n2ccc(=O)[nH]c2=O)C[C@@H]1/C=C/P(=O)(O)O] |
| M-40 | 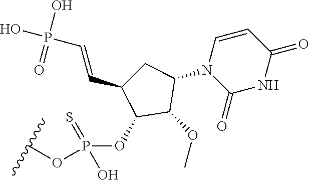 [SMILES: *OP(O)(=S)O[C@H]1[C@@H](OC)[C@@H](n2ccc(=O)[nH]c2=O)C[C@@H]1/C=C/P(=O)(O)O] |
| M-41 | 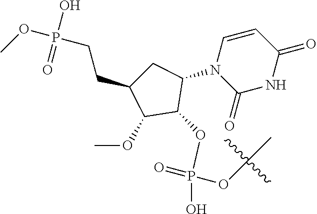 [SMILES: *OP(=O)(O)O[C@@H]1[C@H](OC)[C@@H](CCP(=O)(O)OC)C[C@@H]1n1ccc(=O)[nH]c1=O] |
| M-42 | 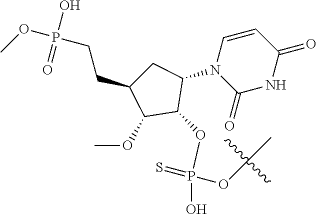 [SMILES: *OP(O)(=S)O[C@@H]1[C@H](OC)[C@@H](CCP(=O)(O)OC)C[C@@H]1n1ccc(=O)[nH]c1=O] |
| M-43 | 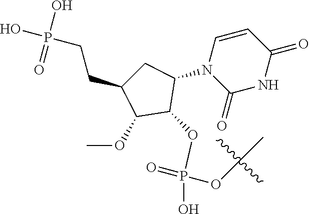 [SMILES: *OP(=O)(O)O[C@@H]1[C@H](OC)[C@@H](CCP(=O)(O)O)C[C@@H]1n1ccc(=O)[nH]c1=O] |
| M-44 | 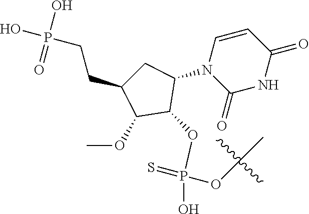 [SMILES: *OP(O)(=S)O[C@@H]1[C@H](OC)[C@@H](CCP(=O)(O)O)C[C@@H]1n1ccc(=O)[nH]c1=O] |
| M-45 | 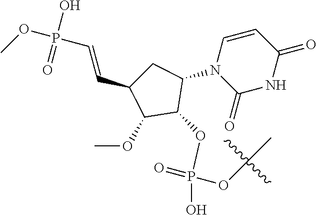 [SMILES: *OP(=O)(O)O[C@@H]1[C@H](OC)[C@@H](/C=C/P(=O)(O)OC)C[C@@H]1n1ccc(=O)[nH]c1=O] |
| M-46 | 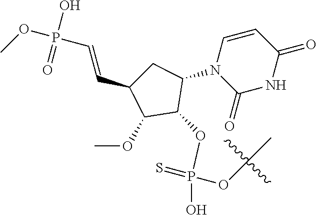 [SMILES: *OP(O)(=S)O[C@@H]1[C@H](OC)[C@@H](/C=C/P(=O)(O)OC)C[C@@H]1n1ccc(=O)[nH]c1=O] |
| M-47 | 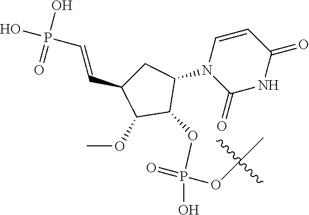 [SMILES: *OP(=O)(O)O[C@@H]1[C@H](OC)[C@@H](/C=C/P(=O)(O)O)C[C@@H]1n1ccc(=O)[nH]c1=O] |
| M-48 | 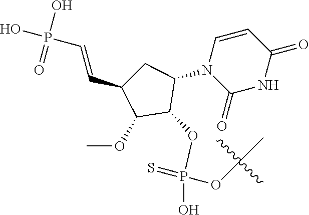 [SMILES: *OP(O)(=S)O[C@@H]1[C@H](OC)[C@@H](/C=C/P(=O)(O)O)C[C@@H]1n1ccc(=O)[nH]c1=O] |

In some embodiments, the phosphate mimic is linked to the 5′-terminus of the isolated oligonucleotides (e.g., siRNAs) as shown in 5′-VP, 5′-C-EP, or 5′-C-MeEP.

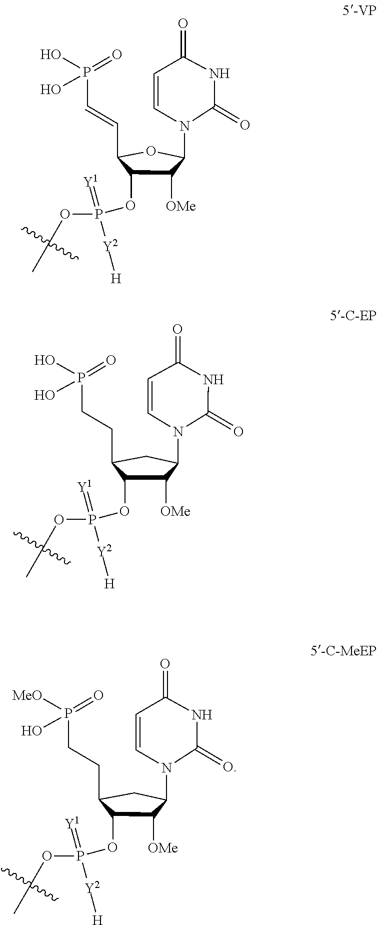

In some embodiments, the phosphate mimic is linked to the 5′-terminus of the isolated oligonucleotides (e.g., siRNAs) as shown in the following formula:

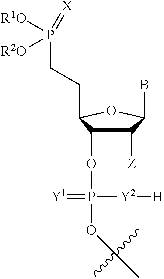

wherein:
B is H or a nucleobase moiety;
X is O or S;
R¹ is H or C₁-C₆ alkyl;
R² is H or C₁-C₆ alkyl;
Y¹ is O or S;
Y² is O or S;
Z is H, halogen, or —OR^{Z};
R^{Z} is H, C₁-C₆ alkyl, or —(C₁-C₆ alkyl)-(C₆-C₁₀ aryl), wherein the C₁-C₆ alkyl or —(C₁-C₆ alkyl)-(C₆-C₁₀ aryl) is optionally substituted with one or more R^{Za};
each R^{Za} independently is halogen, C₁-C₆ alkyl, or —O—(C₁-C₆ alkyl), wherein the C₁-C₆ alkyl or —O—(C₁-C₆ alkyl) is optionally substituted with one or more halogen; and
[symbol] indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

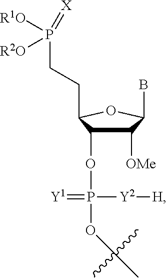

wherein:
B is H or a nucleobase moiety;
X is O or S;
R¹ is H or C₁-C₆ alkyl;
R² is H or C₁-C₆ alkyl;
Y¹ is O or S;
Y² is O or S; and
[symbol] indicates an attachment to a nucleotide of the isolated oligonucleotide (e.g., siRNA).

In some embodiments, the phosphate mimic is linked to the 5′-terminus of the isolated oligonucleotides (e.g., siRNAs) as shown in the following formula:

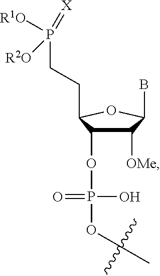

wherein:
B is H or a nucleobase moiety;
X is O or S;
R¹ is H or C₁-C₆ alkyl;
R² is H or C₁-C₆ alkyl; and
[symbol] indicates an attachment to a nucleotide of the isolated oligonucleotide (e.g., siRNA).

In some embodiments, X¹ is —OR^{X}.

In some embodiments, X¹ is —OCH₃.

In some embodiments, Y¹ is O.

In some embodiments, Y¹ is S.

In some embodiments, Y² is O.

In some embodiments, Z¹ is —P(═O)(OR^{Z})₂.

In some embodiments, Z¹ is —P(═O)(OH)₂.

In some embodiments, Z¹ is —P(═O)(OCH₃)(OH).

In some embodiments, R^{1a} is H.

In some embodiments, R^{2a} is H.

In some embodiments, R^{3a} is H.

In some embodiments, R^{4a} is H.

In some embodiments, each R^{6a} is H.

In some embodiments, [symbol] indicates a single bond.

In some embodiments, [symbol] indicates a double bond.

In some embodiments, X is O.

In some embodiments, X is S.

In some embodiments, R¹ is H.

In some embodiments, R¹ is C₁-C₆ alkyl (e.g., methyl, ethyl, n-propyl, i-propyl, n-butyl, i-butyl, s-butyl, t-butyl, pentyl, or hexyl).

In some embodiments, R¹ is methyl.

In some embodiments, R² is H.

In some embodiments, R² is C₁-C₆ alkyl (e.g., methyl, ethyl, n-propyl, i-propyl, n-butyl, i-butyl, s-butyl, t-butyl, pentyl, or hexyl).

In some embodiments, R² is methyl.

In some embodiments, Y¹ is O.

In some embodiments, Y¹ is S.

In some embodiments, Y² is O.

In some embodiments, Y² is S.

In some embodiments, Z is H.

In some embodiments, Z is not H.

In some embodiments, Z is halogen (e.g., F, Cl, Br, or I).

In some embodiments, Z is F or Cl.

In some embodiments, Z is F

In some embodiments, Z is —OR^{Z}.

In some embodiments, Z is —OH.

In some embodiments, Z is not —OH.

In some embodiments, Z is —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, Z is —OCH₃.

In some embodiments, Z is —O—(C₁-C₆ alkyl)-O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl).

In some embodiments, Z is —OCH₂CH₂OCH₃.

In some embodiments, Z is —O—(C₁-C₆ alkyl)-(C₆-C₁₀ aryl) optionally substituted with one or more R^{Za}.

In some embodiments, Z is —O—(C₁-C₆ alkyl)-(C₆-C₁₀ aryl).

In some embodiments, Z is

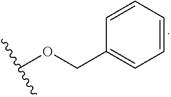 [SMILES: *OCc1ccccc1]

In some embodiments, Z is

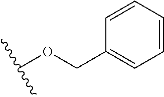 [SMILES: *OCc1ccccc1]

optionally substituted with one or more R^{Za}.

In some embodiments, Z is

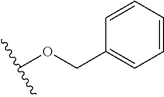 [SMILES: *OCc1ccccc1]

optionally substituted with one or more halogen.

In some embodiments, Z is

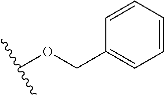 [SMILES: *OCc1ccccc1]

optionally substituted with one or more C₁-C₆ alkyl or —O—(C₁-C₆ alkyl), wherein the C₁-C₆ alkyl or —O—(C₁-C₆ alkyl) is optionally substituted with one or more halogen.

In some embodiments, R^{Z} is H.

In some embodiments, R^{Z} is not H.

In some embodiments, R^{Z} is C₁-C₆ alkyl (e.g., methyl, ethyl, n-propyl, i-propyl, n-butyl, i-butyl, s-butyl, t-butyl, pentyl, or hexyl) optionally substituted with one or more R^{Za}.

In some embodiments, R^{Z} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I) or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl) optionally substituted with one or more halogen.

In some embodiments, R^{Z} is C₁-C₆ alkyl (e.g., methyl, ethyl, n-propyl, i-propyl, n-butyl, i-butyl, s-butyl, t-butyl, pentyl, or hexyl).

In some embodiments, R^{Z} is methyl, ethyl, or propyl.

In some embodiments, R^{Z} is methyl.

In some embodiments, R^{Z} is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, R^{Z} is C₁-C₆ alkyl (e.g., methyl, ethyl, n-propyl, i-propyl, n-butyl, i-butyl, s-butyl, t-butyl, pentyl, or hexyl) substituted with one or more —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the —O—(C₁-C₆ alkyl) is optionally substituted with one or more halogen.

In some embodiments, R^{Z} is —(C₁-C₆ alkyl)-(C₆-C₁₀ aryl) optionally substituted with one or more R^{Za}.

In some embodiments, R^{Z} is —(C₁-C₆ alkyl)-(C₆-C₁₀ aryl) optionally substituted with one or more halogen (e.g., F, Cl, Br, or I), C₁-C₆ alkyl (e.g., methyl, ethyl, n-propyl, i-propyl, n-butyl, i-butyl, s-butyl, t-butyl, pentyl, or hexyl), or —O—(C₁-C₆ alkyl) (e.g., wherein the C₁-C₆ alkyl is methyl, ethyl, n-propyl, i-propyl, n-butyl, i-butyl, s-butyl, t-butyl, pentyl, or hexyl), wherein the C₁-C₆ alkyl or —O—(C₁-C₆ alkyl) is optionally substituted with one or more halogen.

In some embodiments, R^{Z} is —(C₁-C₆ alkyl)-(C₆-C₁₀ aryl).

In some embodiments, at least one R^{Za} is halogen (e.g., F, Cl, Br, or I).

In some embodiments, at least one R^{Za} is F or C₁.

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl) optionally substituted with one or more halogen (e.g., F, Cl, Br, or I).

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl).

In some embodiments, at least one R^{Za} is C₁-C₆ alkyl (e.g., methyl, ethyl, n-propyl, i-propyl, n-butyl, i-butyl, s-butyl, t-butyl, pentyl, or hexyl) substituted with one or more halogen (e.g., F, Cl, Br, or I).

In some embodiments, at least one R^{Za} is —O—(C₁-C₆ alkyl) optionally substituted with one or more halogen (e.g., F, Cl, Br, or I).

In some embodiments, at least one R^{Za} is —O—(C₁-C₆ alkyl).

In some embodiments, at least one R^{Za} is —O—(C₁-C₆ alkyl) substituted with one or more halogen (e.g., F, Cl, Br, or I).

In some embodiments, B is H.

In some embodiments, B is a nucleobase moiety.

The term “nucleobase moiety”, as used herein, refers to a nucleobase that is attached to the rest of the isolated oligonucleotides (e.g., dsRNA or siRNA) of the present disclosure, e.g., via an atom of the nucleobase or a functional group thereof.

In some embodiments, the nucleobase moiety is adenine (A), cytosine (C), guanine (G), thymine (T), or uracil (U).

In some embodiments, the nucleobase moiety is uracil (U).

In some embodiments, the phosphate mimic is linked to the 5′-terminus of the isolated oligonucleotides as shown in the following formula:

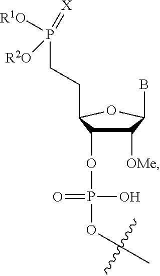

wherein:
B is a nucleobase moiety, wherein the nucleobase moiety is uracil (U), wherein the uracil is at position 1 from the 5′-terminus of the sense strand or at position 1 from the 5′-terminus of the antisense strand;
X is O;
R¹ is C₁ alkyl;
R² is H; and
[symbol] indicates an attachment to a nucleotide of the isolated oligonucleotide (e.g., siRNA).

In some embodiments of the isolated oligonucleotides of the present disclosure, the phosphate mimic is attached to the 5′-terminus of the antisense strand of the isolated oligonucleotide.

In some embodiments, the phosphate mimic is attached to a 5′-terminal uridine of the antisense strand of the isolated oligonucleotide, having the following structure (5′-MeEPmU):

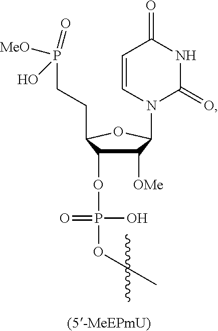

wherein “mU” is a 2′-O-methyl modified uridine nucleotide and “MeEP” is a mono methyl protected phosphate mimic.

In some embodiments, the phosphate mimic is attached to a 5′-terminal uridine of the antisense strand of the isolated oligonucleotide, having the following structure (5′-MeEPmUs):

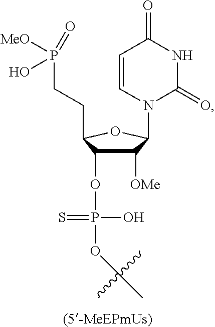

wherein “mU” is a 2′-O-methyl modified uridine nucleotide, “MeEP” is a mono methyl protected phosphate mimic, and “s” is a phosphorothioate internucleotide linkage.

In some embodiments, the phosphate mimic is attached to a 5′-terminal uridine of the antisense strand of the isolated oligonucleotide, having the following structure (5′-EPmUs):

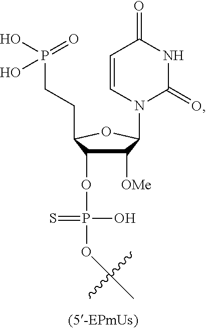

wherein “mU” is a 2′-O-methyl modified uridine nucleotide, “EP” is a phosphate mimic, and “s” is a phosphorothioate internucleotide linkage.

The terms “5′-MeEP”, “5′-MeEP”, and “5′ MeEP” are used interchangeably herein.

In some embodiments, the phosphate mimic is attached to a 5′-terminal uridine of the antisense strand of the isolated oligonucleotide, having the following structure (5′-C-MeEPmU):

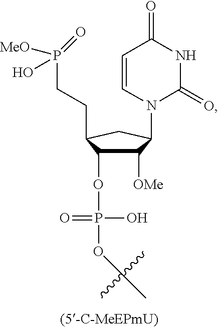

wherein “mU” is a 2′-O-methyl modified uridine nucleotide and “MeEP” is a mono methyl protected phosphate mimic.

In some embodiments, the phosphate mimic is attached to a 5′-terminal uridine of the antisense strand of the isolated oligonucleotide, having the following structure (5′-C-MeEPmUs):

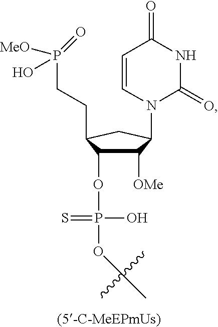

wherein “mU” is a 2′-O-methyl modified uridine nucleotide, “MeEP” is a mono methyl protected phosphate mimic, and “s” is a phosphorothioate internucleotide linkage.

In some embodiments, the phosphate mimic is attached to a 5′-terminal uridine of the antisense strand of the isolated oligonucleotide, having the following structure (5′-C-EPmUs):

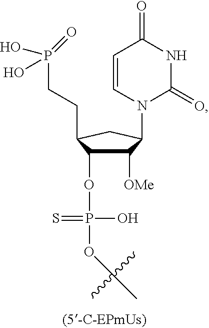

wherein “mU” is a 2′-O-methyl modified uridine nucleotide, “EP” is a phosphate mimic, and “s” is a phosphorothioate internucleotide linkage. In some embodiments, the MeEP is linked to the 5′ end of the antisense strand (5′-MeEP). In some embodiments, the C-MeEP is linked to the 5′ end of the antisense strand (5′-C-MeEP).

In some embodiments, the phosphate mimic is attached to a 5′-terminal uridine of the antisense strand of the isolated oligonucleotide, having the following structure (5′-VPmU):

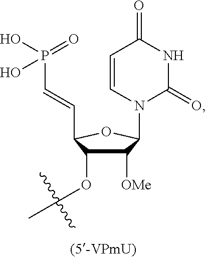

wherein “mU” is a 2′-O-methyl modified uridine nucleotide and “VP” is a vinyl phosphonate modification at the 5′ end.

In some embodiments, the 5′-terminal uridine is a 2′-O-methyl modified nucleotide.

In some embodiments, wherein the MeEP is linked to the 5′ end of the antisense strand, the phosphate mimic is attached to a 5′-terminal uridine of the antisense strand. In some embodiments, wherein the C-MeEP is linked to the 5′ end of the antisense strand, the phosphate mimic is attached to a 5′-terminal uridine of the antisense strand.

In some embodiments, the 5′-terminal uridine is a 2′-O-methyl modified nucleotide.

Suitable phosphate mimics include, but are not limited to, the phosphate mimics disclosed in PCT Application No. PCT/US2023/062996 (PCT Publication No. WO WO 2023/164464) and U.S. application Ser. No. 18/172,719 (US Publication No. US 2023-0346819), each of which is incorporated by reference.

The present disclosure provides an isolated oligonucleotide comprising a sense strand and an antisense strand, wherein the sense strand or the antisense strand or both comprise one or more modified nucleotide(s), and wherein the antisense strand comprises a phosphate mimic. In some embodiments, the phosphate mimic is 5′-C-EP. In some embodiments, the phosphate mimic is 5′-C-EPmUs.

In some embodiments, the isolated oligonucleotide comprises an antisense strand comprising SEQ ID NO: 1074 and its corresponding sense strand comprising SEQ ID NO: 1079, wherein the phosphate mimic is 5′-C-EP. In some embodiments, the isolated oligonucleotide comprises an antisense strand comprising SEQ ID NO: 1074 and its corresponding sense strand comprising SEQ ID NO: 1079, wherein the phosphate mimic is 5′-C-EPmUs.

In some embodiments, the isolated oligonucleotide comprises an antisense strand comprising SEQ ID NO: 1075 and its corresponding sense strand comprising SEQ ID NO: 1079, wherein the phosphate mimic is 5′-C-EP. In some embodiments, the isolated oligonucleotide comprises an antisense strand comprising SEQ ID NO: 1075 and its corresponding sense strand comprising SEQ ID NO: 1079, wherein the phosphate mimic is 5′-C-EPmUs.

In some embodiments, the isolated oligonucleotide comprises an antisense strand comprising SEQ ID NO: 1076 and its corresponding sense strand comprising SEQ ID NO: 1081, wherein the phosphate mimic is 5′-C-EP. In some embodiments, the isolated oligonucleotide comprises an antisense strand comprising SEQ ID NO: 1076 and its corresponding sense strand comprising SEQ ID NO: 1081, wherein the phosphate mimic is 5′-C-EPmUs.

In some embodiments, the isolated oligonucleotide comprises an antisense strand comprising SEQ ID NO: 1077 and its corresponding sense strand comprising SEQ ID NO: 1082, wherein the phosphate mimic is 5′-C-EP. In some embodiments, the isolated oligonucleotide comprises an antisense strand comprising SEQ ID NO: 1077 and its corresponding sense strand comprising SEQ ID NO: 1082, wherein the phosphate mimic is 5′-C-EPmUs.

In some embodiments, the isolated oligonucleotide comprises an antisense strand comprising SEQ ID NO: 1078 and its corresponding sense strand comprising SEQ ID NO: 1083, wherein the phosphate mimic is 5′-C-EP. In some embodiments, the isolated oligonucleotide comprises an antisense strand comprising SEQ ID NO: 1078 and its corresponding sense strand comprising SEQ ID NO: 1083, wherein the phosphate mimic is 5′-C-EPmUs.

In some embodiments, the isolated oligonucleotide comprises an antisense strand comprising SEQ ID NO: 1077 and its corresponding sense strand comprising SEQ ID NO: 1084, wherein the phosphate mimic is 5′-C-EP. In some embodiments, the isolated oligonucleotide comprises an antisense strand comprising SEQ ID NO: 1077 and its corresponding sense strand comprising SEQ ID NO: 1084, wherein the phosphate mimic is 5′-C-EPmUs.

### Modified Backbone Phosphate/Phosphodiester Bond

In some embodiments of the isolated oligonucleotide of the present disclosure, the sense strand or the antisense strand or both comprise at least one nucleotide having a modified phosphate backbone. In some embodiments, the sense strand of the isolated oligonucleotide comprises at least one nucleotide having a modified phosphate backbone. In some embodiments, the antisense strand of the isolated oligonucleotide comprises at least one nucleotide having a modified phosphate backbone. In some embodiments, wherein the isolated oligonucleotide of the present disclosure comprises a modified phosphate backbone, the modified phosphate backbone comprises a modified phosphodiester bond. A phosphodiester bond comprises a linkage having the formula:

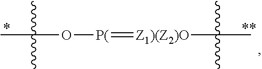

wherein [symbol] denotes attachment to a 3′ carbon of a first nucleotide in the isolated oligonucleotide of the present disclosure; and [symbol] denotes attachment to a 5′ carbon of a second nucleotide in the isolated oligonucleotide of the present disclosure. In some embodiments, the phosphodiester bond is unmodified, wherein Z¹ is O and Z² is OH or O⁻. In some embodiments, the phosphodiester bond is modified, wherein Z¹ is O, S, NH, or N(C₁-C₆ alkyl) and Z² is OH, SH, NH₂, NH(C₁-C₆ alkyl), O⁻, S⁻, HN⁻, or (C₁-C₆ alkyl)N⁻, and wherein when Z¹ is O, Z² is not OH or O⁻.

In some embodiments, Z₁ is O.

In some embodiments, Z₁ is S.

In some embodiments, Z₁ is NH.

In some embodiments, Z₁ is N(C₁-C₆ alkyl).

In some embodiments, Z₂ is OH.

In some embodiments, Z₂ is SH.

In some embodiments, Z₂ is NH₂.

In some embodiments, Z₂ is NH(C₁-C₆ alkyl).

In some embodiments, Z₂ is SH, NH₂, or NH(C₁-C₆ alkyl).

In some embodiments, Z₂ is O⁻.

In some embodiments, Z₂ is S⁻.

In some embodiments, Z₂ is HN⁻.

In some embodiments, Z₂ is (C₁-C₆ alkyl)N⁻.

In some embodiments, Z₂ is S⁻, HN⁻, or (C₁-C₆ alkyl)N⁻.

In some embodiments, Z₁ is O and Z₂ is SH.

In some embodiments, Z₁ is O and Z₂ is NH₂.

In some embodiments, Z₁ is O and Z₂ is NH(C₁-C₆ alkyl).

In some embodiments, Z₁ is S and Z₂ is OH.

In some embodiments, Z₁ is S and Z₂ is SH.

In some embodiments, Z₁ is S and Z₂ is NH₂.

In some embodiments, Z₁ is S and Z₂ is NH(C₁-C₆ alkyl).

In some embodiments, Z₁ is NH and Z₂ is OH.

In some embodiments, Z₁ is NH and Z₂ is SH.

In some embodiments, Z₁ is NH and Z₂ is NH₂.

In some embodiments, Z₁ is NH and Z₂ is NH(C₁-C₆ alkyl).

In some embodiments, Z₁ is N(C₁-C₆ alkyl) and Z₂ is OH.

In some embodiments, Z₁ is N(C₁-C₆ alkyl) and Z₂ is SH.

In some embodiments, Z₁ is N(C₁-C₆ alkyl) and Z₂ is NH₂.

In some embodiments, Z₁ is N(C₁-C₆ alkyl) and Z₂ is NH(C₁-C₆ alkyl).

In some embodiments, Z₁ is O and Z₂ is S⁻.

In some embodiments, Z₁ is O and Z₂ is HN⁻.

In some embodiments, Z₁ is O and Z₂ is (C₁-C₆ alkyl)N⁻.

In some embodiments, Z₁ is S and Z₂ is O⁻.

In some embodiments, Z₁ is S and Z₂ is S⁻.

In some embodiments, Z₁ is S and Z₂ is HN⁻.

In some embodiments, Z₁ is S and Z₂ is (C₁-C₆ alkyl)N⁻.

In some embodiments, Z₁ is NH and Z₂ is O⁻.

In some embodiments, Z₁ is NH and Z₂ is S⁻.

In some embodiments, Z₁ is NH and Z₂ is HN⁻.

In some embodiments, Z₁ is NH and Z₂ is (C₁-C₆ alkyl)N⁻.

In some embodiments, Z₁ is N(C₁-C₆ alkyl) and Z₂ is O⁻.

In some embodiments, Z₁ is N(C₁-C₆ alkyl) and Z₂ is S⁻.

In some embodiments, Z₁ is N(C₁-C₆ alkyl) and Z₂ is HN⁻.

In some embodiments, Z₁ is N(C₁-C₆ alkyl) and Z₂ is (C₁-C₆ alkyl)N⁻.

In some embodiments, the modified phosphodiester bond comprises a phosphorothioate internucleotide linkage.

In some embodiments, the modified phosphodiester bond comprises

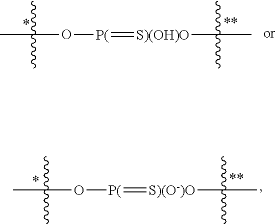

wherein [symbol] denotes attachment to a 3′ carbon of a first nucleotide in the isolated oligonucleotide of the present disclosure; and [symbol] denotes attachment to a 5′ carbon of a second nucleotide in the isolated oligonucleotide of the present disclosure.

In some embodiments, the modified phosphodiester bond comprises

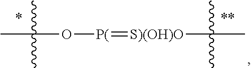

wherein [symbol] denotes attachment to a 3′ carbon of a first nucleotide in the isolated oligonucleotide of the present disclosure; and [symbol] denotes attachment to a 5′ carbon of a second nucleotide in the isolated oligonucleotide of the present disclosure.

In some embodiments, the modified phosphodiester bond comprises

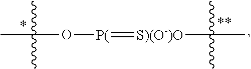

wherein [symbol] denotes attachment to a 3′ carbon of a first nucleotide in the isolated oligonucleotide of the present disclosure; and [symbol] denotes attachment to a 5′ carbon of a second nucleotide in the isolated oligonucleotide of the present disclosure.

In some embodiments, the isolated oligonucleotide of the present disclosure comprises at least one modified phosphodiester bond(s). In some embodiments of the isolated oligonucleotide of the present disclosure, the sense strand or the antisense strand or both comprise one or more modified phosphodiester bonds. In some embodiments, only the sense strand comprises one or more modified phosphodiester bonds. In some embodiments, only the antisense strand comprises one or more modified phosphodiester bonds. In some embodiments, both the sense strand and antisense strand comprise one or more modified phosphodiester bonds.

In some embodiments, the isolated oligonucleotide comprises at least two modified phosphodiester bonds. In some embodiments, the isolated oligonucleotide comprises at least three modified phosphodiester bonds. In some embodiments, the isolated oligonucleotide comprises at least four modified phosphodiester bonds. In some embodiments, the isolated oligonucleotide comprises at least five modified phosphodiester bonds. In some embodiments, the isolated oligonucleotide comprises at least six modified phosphodiester bonds. In some embodiments, the isolated oligonucleotide comprises at least seven modified phosphodiester bonds. In some embodiments, the isolated oligonucleotide comprises at least eight modified phosphodiester bonds. In some embodiments, the isolated oligonucleotide comprises at least nine modified phosphodiester bonds. In some embodiments, the isolated oligonucleotide comprises at least ten modified phosphodiester bonds. In some embodiments, the isolated oligonucleotide comprises at least eleven modified phosphodiester bonds. In some embodiments, the isolated oligonucleotide comprises at least twelve modified phosphodiester bonds. In some embodiments, the isolated oligonucleotide comprises at least thirteen modified phosphodiester bonds. In some embodiments, the isolated oligonucleotide comprises at least fourteen modified phosphodiester bonds. In some embodiments, the isolated oligonucleotide comprises at least fifteen modified phosphodiester bonds. some embodiments, the isolated oligonucleotide comprises at least sixteen modified phosphodiester bonds. In some embodiments, the isolated oligonucleotide comprises at least seventeen modified phosphodiester bonds. In some embodiments, the isolated oligonucleotide comprises at least eighteen modified phosphodiester bonds. In some embodiments, the isolated oligonucleotide comprises at least nineteen modified phosphodiester bonds. In some embodiments, the isolated oligonucleotide comprises at least twenty modified phosphodiester bonds. In some embodiments, the isolated oligonucleotide comprises more than twenty modified phosphodiester bonds. In some embodiments, the isolated oligonucleotide comprises between twenty and thirty modified phosphodiester bonds. In some embodiments, the isolated oligonucleotide comprises between thirty and forty modified phosphodiester bonds. In some embodiments, the isolated oligonucleotide comprises between forty and fifty modified phosphodiester bonds.

In some embodiments, the isolated oligonucleotide comprises at least two phosphorothioate internucleotide linkages. In some embodiments, the isolated oligonucleotide comprises at least three phosphorothioate internucleotide linkages. In some embodiments, the isolated oligonucleotide comprises at least four phosphorothioate internucleotide linkages. In some embodiments, the isolated oligonucleotide comprises at least five phosphorothioate internucleotide linkages. In some embodiments, the isolated oligonucleotide comprises at least six phosphorothioate internucleotide linkages. In some embodiments, the isolated oligonucleotide comprises at least seven phosphorothioate internucleotide linkages. In some embodiments, the isolated oligonucleotide comprises at least eight phosphorothioate internucleotide linkages. In some embodiments, the isolated oligonucleotide comprises at least nine phosphorothioate internucleotide linkages. In some embodiments, the isolated oligonucleotide comprises at least ten phosphorothioate internucleotide linkages. In some embodiments, the isolated oligonucleotide comprises at least eleven phosphorothioate internucleotide linkages. In some embodiments, the isolated oligonucleotide comprises at least twelve phosphorothioate internucleotide linkages. In some embodiments, the isolated oligonucleotide comprises at least thirteen phosphorothioate internucleotide linkages. In some embodiments, the isolated oligonucleotide comprises at least fourteen phosphorothioate internucleotide linkages. In some embodiments, the isolated oligonucleotide comprises at least fifteen phosphorothioate internucleotide linkages. In some embodiments, the isolated oligonucleotide comprises at least sixteen phosphorothioate internucleotide linkages. In some embodiments, the isolated oligonucleotide comprises at least seventeen phosphorothioate internucleotide linkages. In some embodiments, the isolated oligonucleotide comprises at least eighteen phosphorothioate internucleotide linkages. In some embodiments, the isolated oligonucleotide comprises at least nineteen phosphorothioate internucleotide linkages. In some embodiments, the isolated oligonucleotide comprises at least twenty phosphorothioate internucleotide linkages. In some embodiments, the isolated oligonucleotide comprises more than twenty phosphorothioate internucleotide linkages. In some embodiments, the isolated oligonucleotide comprises between twenty and thirty phosphorothioate internucleotide linkages. In some embodiments, the isolated oligonucleotide comprises between thirty and forty phosphorothioate internucleotide linkages. In some embodiments, the isolated oligonucleotide comprises between forty and fifty phosphorothioate internucleotide linkages.

In some embodiments, the sense strand and/or the antisense strand of the isolated oligonucleotide each comprise at least one modified phosphodiester bond(s). In some embodiments, the sense strand and/or the antisense strand of the isolated oligonucleotide each comprise at least two modified phosphodiester bonds. In some embodiments, the sense strand and/or the antisense strand of the isolated oligonucleotide each comprise at least three modified phosphodiester bonds. In some embodiments, the sense strand and/or the antisense strand of the isolated oligonucleotide each comprise at least four modified phosphodiester bonds. In some embodiments, the sense strand and/or the antisense strand of the isolated oligonucleotide each comprise at least five modified phosphodiester bonds. In some embodiments, the sense strand and/or the antisense strand of the isolated oligonucleotide each comprise at least six modified phosphodiester bonds. In some embodiments, the sense strand and/or the antisense strand of the isolated oligonucleotide each comprise at least seven modified phosphodiester bonds. In some embodiments, the sense strand and/or the antisense strand of the isolated oligonucleotide each comprise at least eight modified phosphodiester bonds. In some embodiments, the sense strand and/or the antisense strand of the isolated oligonucleotide each comprise at least nine modified phosphodiester bonds. In some embodiments, the sense strand and/or the antisense strand of the isolated oligonucleotide each comprise at least ten modified phosphodiester bonds. In some embodiments, the sense strand and/or the antisense strand of the isolated oligonucleotide each comprise at least eleven modified phosphodiester bonds. In some embodiments, the sense strand and/or the antisense strand of the isolated oligonucleotide each comprise at least twelve modified phosphodiester bonds. In some embodiments, the sense strand and/or the antisense strand of the isolated oligonucleotide each comprise at least thirteen modified phosphodiester bonds. In some embodiments, the sense strand and/or the antisense strand of the isolated oligonucleotide each comprise at least fourteen modified phosphodiester bonds. In some embodiments, the sense strand and/or the antisense strand of the isolated oligonucleotide each comprise at least fifteen modified phosphodiester bonds. In some embodiments, the sense strand and/or the antisense strand of the isolated oligonucleotide each comprise at least sixteen modified phosphodiester bonds. In some embodiments, the sense strand and/or the antisense strand of the isolated oligonucleotide each comprise at least seventeen modified phosphodiester bonds. In some embodiments, the sense strand and/or the antisense strand of the isolated oligonucleotide each comprise at least eighteen modified phosphodiester bonds. In some embodiments, the sense strand and/or the antisense strand of the isolated oligonucleotide each comprise at least nineteen modified phosphodiester bonds. In some embodiments, the sense strand and/or the antisense strand of the isolated oligonucleotide each comprise at least twenty modified phosphodiester bonds.

In some embodiments, the sense strand and/or the antisense strand of the isolated oligonucleotide each comprise at least one phosphorothioate internucleotide linkage(s). In some embodiments, the sense strand and/or the antisense strand of the isolated oligonucleotide each comprise at least two phosphorothioate internucleotide linkages. In some embodiments, the sense strand and/or the antisense strand of the isolated oligonucleotide each comprise at least three phosphorothioate internucleotide linkages. In some embodiments, the sense strand and/or the antisense strand of the isolated oligonucleotide each comprise at least four phosphorothioate internucleotide linkages. In some embodiments, the sense strand and/or the antisense strand of the isolated oligonucleotide each comprise at least five phosphorothioate internucleotide linkages. In some embodiments, the sense strand and/or the antisense strand of the isolated oligonucleotide each comprise at least six phosphorothioate internucleotide linkages. In some embodiments, the sense strand and/or the antisense strand of the isolated oligonucleotide each comprise at least seven phosphorothioate internucleotide linkages. In some embodiments, the sense strand and/or the antisense strand of the isolated oligonucleotide each comprise at least eight phosphorothioate internucleotide linkages. In some embodiments, the sense strand and/or the antisense strand of the isolated oligonucleotide each comprise at least nine phosphorothioate internucleotide linkages. In some embodiments, the sense strand and/or the antisense strand of the isolated oligonucleotide each comprise at least ten phosphorothioate internucleotide linkages. In some embodiments, the sense strand and/or the antisense strand of the isolated oligonucleotide each comprise at least eleven phosphorothioate internucleotide linkages. In some embodiments, the sense strand and/or the antisense strand of the isolated oligonucleotide each comprise at least twelve phosphorothioate internucleotide linkages. In some embodiments, the sense strand and/or the antisense strand of the isolated oligonucleotide each comprise at least thirteen phosphorothioate internucleotide linkages. In some embodiments, the sense strand and/or the antisense strand of the isolated oligonucleotide each comprise at least fourteen phosphorothioate internucleotide linkages. In some embodiments, the sense strand and/or the antisense strand of the isolated oligonucleotide each comprise at least fifteen phosphorothioate internucleotide linkages. In some embodiments, the sense strand and/or the antisense strand of the isolated oligonucleotide each comprise at least sixteen phosphorothioate internucleotide linkages. In some embodiments, the sense strand and/or the antisense strand of the isolated oligonucleotide each comprise at least seventeen phosphorothioate internucleotide linkages. In some embodiments, the sense strand and/or the antisense strand of the isolated oligonucleotide each comprise at least eighteen phosphorothioate internucleotide linkages. In some embodiments, the sense strand and/or the antisense strand of the isolated oligonucleotide each comprise at least nineteen phosphorothioate internucleotide linkages. In some embodiments, the sense strand and/or the antisense strand of the isolated oligonucleotide each comprise at least twenty phosphorothioate internucleotide linkages.

In some embodiments, the modified phosphodiester bonds are consecutively located on the sense strand or the antisense strand or both. In some embodiments, some but not all of the modified phosphodiester bonds are consecutively located on the sense strand or the antisense strand or both. In some embodiments, the modified phosphodiester bonds on the sense strand or the antisense strand or both are not consecutively located.

Envisaged within the present disclosure is an isolated oligonucleotide, wherein any phosphodiester bond on the sense strand or antisense strand can be modified. In some embodiments, any phosphodiester bond on the antisense strand can be modified. In some embodiments, any phosphodiester bond on the antisense strand can be modified.

In some embodiments of the isolated oligonucleotide of the present disclosure, the antisense strand comprises between one and twenty, between one and fifteen, between one and ten, between one and five, or less than five modified phosphodiester bonds. In some embodiments, the between one and twenty, between one and fifteen, between one and ten, between one and five, or less than five modified phosphodiester bonds comprise phosphorothioate internucleotide linkages. In some embodiments, the antisense strand comprises less than five modified phosphodiester bonds. In some embodiments, the antisense strand comprises one, two, three, or four modified phosphodiester bonds. In some embodiments, wherein the antisense strand comprises one, two, three, or four modified phosphodiester bonds, the one, two, three, or four modified phosphodiester bonds comprise phosphorothioate internucleotide linkages. In some embodiments, the antisense strand comprises four modified phosphodiester bonds. In some embodiments, wherein the antisense strand comprises four modified phosphodiester bonds. the modified phosphodiester bonds comprise phosphorothioate.

In some embodiments, wherein the antisense strand comprises at least one, at least two, at least three, or at least four phosphorothioate internucleotide linkages, the phosphorothioate internucleotide linkages connect the nucleotides at position 1 and position 2 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 2 and position 3 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 20 and position 21 from the first nucleotide at the 5′-terminus of the antisense strand. In some embodiments, wherein the antisense strand comprises at least one, at least two, at least three, or at least four phosphorothioate internucleotide bonds, the phosphorothioate internucleotide linkages connect the nucleotides at position 21 and position 22 from the first nucleotide at the 5′-terminus of the antisense strand. In some embodiments, wherein the antisense strand comprises at least one, at least two, at least three, or at least four modified phosphodiester bonds, wherein the modified phosphodiester bonds comprise phosphorothioate internucleotide linkages, the phosphorothioate internucleotide linkages are located between nucleotides at position 1 and 2, position 2 and 3, position 20 and 21, and position 21 and 22 from the first nucleotide at the 5′-terminus of the antisense strand.

In some embodiments of the isolated oligonucleotide of the present disclosure, wherein the antisense strand comprises at least one, at least two, at least three, or at least four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, wherein the antisense strand comprises at least four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, the antisense strand comprises four phosphorothioate internucleotide linkages. In some embodiments, wherein the antisense strand comprises four phosphorothioate internucleotide linkages, the phosphorothioate internucleotide linkages are located between nucleotides at position 1 to 3 and nucleotides at position 20 to 22 from the first nucleotide at the 5′-terminus of the antisense strand.

In some embodiments of the isolated oligonucleotide of the present disclosure, the sense strand comprises between one and twenty, between one and fifteen, between one and ten, between one and five, or less than five modified phosphodiester bonds. In some embodiments, the between one and twenty, between one and fifteen, between one and ten, between one and five, or less than five modified phosphodiester bonds comprise phosphorothioate internucleotide linkages. In some embodiments, the sense strand comprises less than five modified phosphodiester bonds. In some embodiments, wherein the sense strand comprises less than five modified phosphodiester bonds, the sense strand comprises one, two, three, or four modified phosphodiester bonds. In some embodiments, wherein the sense strand comprises one, two, three, or four modified phosphodiester bonds, the one, two, three, or four modified phosphodiester bonds comprise phosphorothioate internucleotide linkages. In some embodiments, the sense strand comprises four modified phosphodiester bonds. In some embodiments, wherein the sense strand comprises four modified phosphodiester bonds, the modified phosphodiester bonds comprise phosphorothioate internucleotide linkages.

In some embodiments, wherein the sense strand comprises at least one, at least two, at least three, or at least four modified phosphodiester bonds, the phosphodiester bonds comprise phosphorothioate internucleotide linkages. In some embodiments, wherein the sense strand comprises at least one, at least two, at least three, or at least four phosphorothioate internucleotide linkages, the phosphorothioate internucleotide linkages connect the nucleotides at position 1 and position 2 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 2 and position 3 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 18 and position 19 from the first nucleotide at the 5′-terminus of the sense strand. In some embodiments, wherein the sense strand comprises at least one, at least two, at least three, or at least four phosphorothioate internucleotide linkages, the phosphorothioate internucleotide linkages connect the nucleotides at position 19 and position 20 from the first nucleotide at the 5′-terminus of the sense strand. In some embodiments, wherein the sense strand comprises at least one, at least two, at least three, or at least four modified phosphodiester bonds, wherein the modified phosphodiester bonds comprise phosphorothioate internucleotide linkages, the phosphorothioate internucleotide linkages are located between nucleotides at position 1 and 2, position 2 and 3, position 18 and 19, and position 19 and 20 from the first nucleotide at the 5′-terminus of the sense strand.

In some embodiments of the isolated oligonucleotide of the present disclosure, wherein the sense strand comprises at least one, at least two, at least three, or at least four phosphorothioate internucleotide linkages, the phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, wherein the sense strand comprises at least four phosphorothioate internucleotide linkages, the at least four phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, the sense strand comprises four phosphorothioate internucleotide linkages. In some embodiments, wherein the sense strand comprises four phosphorothioate internucleotide linkages, the phosphorothioate internucleotide linkages are located between nucleotides at position 1 to 3 and nucleotides at position 18 to 20 from the first nucleotide at the 5′-terminus of the sense strand.

In some embodiments of the isolated oligonucleotide of the present disclosure, wherein the sense strand comprises at least one, at least two, or at least three internucleotide linkages, the phosphorothioate internucleotide linkages are located between nucleotides at position 1 to 3 and nucleotides at position 19 to 20 from the first nucleotide at the 5′-terminus of the sense strand.

In some embodiments of the isolated oligonucleotide of the present disclosure, wherein the sense strand comprises at least three phosphorothioate internucleotide linkages, the at least three phosphorothioate internucleotide linkages are located between nucleotides at position 1 to 3 and nucleotides at position 19 to 20 from the first nucleotide at the 5′-terminus of the sense strand.

In some embodiments of the isolated oligonucleotide of the present disclosure, the sense strand comprises three phosphorothioate internucleotide linkages. In some embodiments, wherein the sense strand comprises three phosphorothioate internucleotide linkages, the phosphorothioate internucleotide linkages are located between nucleotides at position 1 to 3 and nucleotides at position 19 to 20 from the first nucleotide at the 5′-terminus of the sense strand.

In some embodiments of the isolated oligonucleotide of the present disclosure, wherein the antisense strand and the sense strand comprise four phosphorothioate internucleotide linkages, the antisense strand comprises phosphorothioate internucleotide linkages located between nucleotides at position 1 to 3 and nucleotides at position 20 to 22 from the first nucleotide at the 5′-terminus of the antisense strand, and the sense strand comprises phosphorothioate internucleotide linkages located between nucleotides at position 1 to 3 and nucleotides at position 18 to 20 from the first nucleotide at the 5′-terminus of the sense strand.

In some embodiments of the isolated oligonucleotide of the present disclosure, wherein the antisense strand and the sense strand comprise four phosphorothioate internucleotide linkages, the antisense strand comprises phosphorothioate internucleotide linkages located between nucleotides at position 1 to 3 and nucleotides at position 20 to 22 from the first nucleotide at the 5′-terminus of the antisense strand, and the sense strand comprises phosphorothioate internucleotide linkages located between nucleotides at position 1 to 3 and nucleotides at position 19 to 20 from the first nucleotide at the 5′-terminus of the sense strand.

### Nucleic Acids and Vectors

The present disclosure also provides a vector encoding at least one isolated oligonucleotide disclosed herein. In some embodiments, the vector is any one of a plasmid, a cosmid, or a viral vector. In some embodiments, the vector is an adenoviral vector. In some embodiments, the vector is a lentiviral vector. In some embodiments, the plasmid is an expression plasmid. In some embodiments, the vector encodes one isolated oligonucleotide disclosed herein. In some embodiments, the vector encodes more than one isolated oligonucleotide disclosed herein. In some embodiments, the vector encodes, two, three, four, or five isolated oligonucleotides disclosed herein. In some embodiments, the vector encodes more than five isolated oligonucleotides disclosed herein.

The disclosure provides nucleic acids comprising the sequences encoding the isolated oligonucleotides (e.g., dsRNAs or siRNAs) targeting ACVR1C described herein.

In some embodiments, the nucleic acids are ribonucleic acids (RNAs). In some embodiments, the nucleic acids are deoxyribonucleic acids (DNAs). The DNAs may be a vector or a plasmid, e.g., an expression vector.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

By the term “express” or “expression” of a polynucleotide coding sequence, it is meant that the sequence is transcribed, and optionally, translated. Typically, according to the present disclosure, expression of a coding sequence of the disclosure will result in production of the polypeptide of the disclosure. The entire expressed polypeptide or fragment can also function in intact cells without purification.

In some embodiments, the vector is an expression vector for manufacturing siRNAs of the disclosure. Exemplary expression vectors may comprise a sequence encoding the sense and/or antisense strand of the isolated oligonucleotide of the present disclosure, under the control of a suitable promoter for transcription. Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

The isolated oligonucleotide of the present disclosure (e.g., dsRNAs and siRNAs) can be expressed endogenously from plasmid or viral expression vectors, or from minimal expression cassettes, for example, PCR generated fragments comprising one or more promoters and an appropriate template or templates for transcribing the siRNA. Examples of commercially available plasmid-based expression vectors for shRNA include members of the pSilencer series (Ambion) and pCpG-siRNA (InvivoGen). Examples of kits for production of PCR-generated shRNA expression cassettes include Silencer Express (Ambion) and siXpress (Mirus)

Viral vectors for the in vivo expression of the isolated oligonucleotides (e.g., siRNAs and dsRNAs) in eukaryotic cells are also contemplated as within the scope of the instant disclosure. Viral vectors may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion) and pLenti6/BLOCK-iT™-DEST (Invitrogen). Selection of viral vectors, methods for expressing the siRNA from the vector and methods of delivering the viral vector, for example incorporated within a nanoparticle, are within the ordinary skill of one in the art.

It will be apparent to those skilled in the art that any suitable vector, optionally incorporated into a nanoparticle, can be used to deliver the isolated oligonucleotides of the present disclosure (e.g., dsRNAs or siRNAs) described herein to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

### Delivery Systems

The present disclosure also provides a delivery system comprising at least one isolated oligonucleotide disclosed herein or vector of the present disclosure encoding at least one isolated oligonucleotide disclosed herein. In some embodiments, the delivery system is any one of a liposome, a nanoparticle, a polymer based delivery system or a ligand-conjugate delivery system. In some embodiments, the ligand-conjugate delivery system comprises one or more of an antibody, a peptide, a lipid, a sugar moiety or a combination thereof.

In some embodiments, the delivery system of the present disclosure comprises nanoparticles comprising the isolated oligonucleotides of the present disclosure (e.g., siRNA or dsRNAs) targeting a ACVR1C mRNA for degradation.

In some embodiments, the delivery system comprises a liposome. Liposomes are spherical vesicles having at least one lipid bilayer, and in some embodiments, an aqueous core. Examples of lipidoids and lipid-based formulations are provided in U.S. Published Application 20090023673. In other embodiments, the one or more lipids are one or more cationic lipids. One skilled in the art will recognize which liposomes are appropriate for siRNA encapsulation.

In some embodiments, the liposome or the nanoparticle of the present disclosure comprises a micelle.

In some embodiments of the isolated oligonucleotides of the present disclosure, the delivery system comprises a ligand-conjugate delivery system. In some embodiments, the ligand-conjugate delivery system comprises one or more of an antibody, a peptide, a sugar moiety, lipid or a combination thereof.

In some embodiments, the ligand conjugate delivery system further comprises a targeting agent. In some embodiments, the targeting agent is an adipose-targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment.

In some embodiments, the isolated oligonucleotide disclosed herein may further comprise a ligand that facilitates delivery or uptake of the isolated oligonucleotide to a particular tissue or cell, such as a liver cell or an adipose cell. In certain embodiments, the ligand targets delivery of the RNAi construct to hepatocytes. The ligand can be covalently attached to the 5′ or 3′ end of the sense strand of the RNAi construct, optionally via a linker.

In some embodiments, the targeting agent comprises a binding partner for a cell surface protein that is upregulated or overexpressed or normally expressed in a target cell encoding ACVR1C mRNA and expressing ACVR1C. In some embodiments, the binding partner can be a transmembrane peptidoglycan expressed on the surface of many types of such cells. Targeting of cell surface protein by the delivery system of the present disclosure thus provides superior delivery and specificity of the compositions of the disclosure to target cells. In some embodiments, the target cell can be any one of an intestinal cell, an arterial cell, a cell of the cardiovascular system, a hepatocyte, a pancreatic cell or a combination thereof.

In some embodiments, the delivery system of the present disclosure comprises a polymer based delivery system. In some embodiments, polymer based delivery system comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In other embodiments, the polyalkylene oxide is polyethylene oxide (PEO).

In some embodiments, the delivery system of the present disclosure is a polymer-based nanoparticle. Polymer based nanoparticles comprise one or more polymers. In some embodiments, the one or more polymers comprise a polyester, poly(ortho ester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In some embodiments, the one or more polymers comprise poly(lactic-co-glycolic acid) (PLGA). In some embodiments, the one or more polymers comprise poly(lactic acid) (PLA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the polymer-based nanoparticle comprises poly(lactic-co-glycolic acid) PLGA polymers. In some embodiments, the PLGA nanoparticle further comprises a targeting agent, as described herein.

In some embodiments, the delivery system of the present disclosure is a nanoparticle of average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

### Therapeutic Agents

In some embodiments, the delivery system of the present disclosure is administered with one or more additional therapeutic agents. In some embodiments, the additional therapeutic agents can be a steroid, an anti-inflammatory agent, an antibody, a fusion protein, a small molecule, or combination thereof. In some embodiments, the additional therapeutic agent is incorporated into a delivery system of the present disclosure comprising at least one isolated oligonucleotide targeting ACVR1C disclosed herein. In some embodiments, the additional therapeutic agent is conjugated to, complexed to, or encapsulated by the one or more lipids or polymers of the delivery system. Additional therapeutic agents can be encapsulated in the hollow core of delivery system. Alternatively, additionally, additional therapeutic agents can be incorporated into the lipid or polymer-based shell of the delivery system, for example via intercalation. Alternatively, additionally, additional therapeutic agents can be attached to the surface of the delivery system. In some embodiments, the additional therapeutic agents are conjugated to one or more lipids or polymers of the delivery system, e.g. via covalent attachment.

In some embodiments, the additional therapeutic agent and the delivery system comprising at least one isolated oligonucleotide targeting ACVR1C disclosed herein are formulated in the same composition. For example, the delivery system comprising at least one isolated oligonucleotide of the present disclosure targeting ACVR1C and the additional therapeutic agent can be formulated in the same pharmaceutical composition.

In some embodiments, the additional therapeutic agent and the delivery system comprising at least one isolated oligonucleotide targeting ACVR1C disclosed herein are formulated as separate compositions, e.g., for separate administration to a subject.

### Pharmaceutical Compositions

The present disclosure also provides a pharmaceutical composition comprising at least one oligonucleotide disclosed herein, a vector of the present disclosure encoding at least one isolated oligonucleotide disclosed herein, or a delivery system of the present disclosure, and a pharmaceutically acceptable carrier, diluent, or excipient.

The pharmaceutical compositions of the disclosure can optionally comprise therapeutic agents, pharmaceutical agents, carriers, adjuvants, dispersing agents, diluents, and the like. In some embodiments, the pharmaceutical composition comprises a therapeutic agent, such as a chemotherapeutic agent. In some embodiments, the therapeutic agent is formulated in the delivery system comprising the one or more isolated oligonucleotides (e.g., dsRNA or siRNA) targeting ACVR1C of the present disclosure.

In some embodiments, an additional therapeutic agent is not formulated in the delivery system comprising the one or more isolated oligonucleotides (e.g., dsRNA or siRNA) targeting ACVR1C of the present disclosure, but both the delivery system and the therapeutic agent are formulated in the same pharmaceutical composition. In some embodiments, an additional therapeutic agent is not formulated in the delivery system comprising the one or more isolated oligonucleotides (e.g., dsRNA or siRNA) targeting ACVR1C of the present disclosure, and the delivery system and the therapeutic agent are formulated in separate pharmaceutical compositions.

Pharmaceutical compositions of the present disclosure can contain any of the reagents discussed above, and one or more of a pharmaceutically acceptable carrier, a diluent or an excipient.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids.

Techniques for formulation and administration of the disclosed compositions of the disclosure are well known to those skilled in the art.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### Methods of Making Isolated Oligonucleotides

Provided herein are methods of making the one or more oligonucleotides of (e.g., dsRNAs or siRNAs) targeting ACVR1C of the present disclosure and delivery systems comprising same.

The one or more oligonucleotides of (e.g., dsRNAs or siRNAs) targeting ACVR1C of the present disclosure, may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers. The siRNAs can be purified by extraction with a solvent or resin, precipitation, electrophoresis, chromatography, or a combination thereof, for example. Alternatively, siRNAs may be used with little if any purification to avoid losses due to sample processing.

In some embodiments, the one or more oligonucleotides of (e.g., dsRNAs or siRNAs) targeting ACVR1C of the present disclosure can be produced using an expression vector into which a nucleic acid encoding the double stranded RNA has been cloned, for example under control of a suitable promoter.

In some embodiments, the one or more oligonucleotides of (e.g., dsRNAs or siRNAs) targeting ACVR1C of the present disclosure can be incorporated in a delivery system of the present disclosure (e.g., a nanoparticle).

Delivery systems comprising dsRNAs or siRNAs of the disclosure can be prepared by any suitable means known in the art. For example, polymeric nanoparticles can be prepared using various methods including, but not limited to, solvent evaporation, spontaneous emulsification, solvent diffusion, desolation, dialysis, ionic gelation, nanoprecipitation, salting out, spray drying and supercritical fluid methods. The dispersion of preformed polymers and the polymerization of monomers are two additional strategies for preparation of polymeric nanoparticles. However, the choice of an appropriate method depends upon various factors, which will be known to the person of ordinary skill in the art.

Sterile injectable solutions comprising a delivery system of the disclosure can be prepared by incorporating the one or more isolated oligonucleotides (e.g. dsRNA and siRNA) targeting ACVR1C disclosed herein, in the delivery systems (e.g. nanoparticle) in the required amount in an appropriate solvent with one or a combination of ingredients enumerated herein, as required, followed by filtered sterilization. Alternatively, or in addition, sterilization can be achieved through other means such as radiation or gas. Generally, dispersions are prepared by incorporating the delivery particles into a sterile vehicle that contains a basic dispersion medium and the required other ingredients from those enumerated above. In the case of sterile powders for the preparation of sterile injectable solutions, methods of preparation are vacuum drying and freeze drying that yields a powder of delivery system comprising the one or more isolated oligonucleotides (e.g. dsRNA and siRNA) targeting ACVR1C disclosed herein, plus any additional desired ingredient from a previously sterile filtered solution thereof.

### Methods of Use

The present disclosure also provides a method of inhibiting or downregulating the expression or level of ACVR1C in a subject in need thereof, wherein the method comprises administering to the subject an effective amount at least one isolated oligonucleotide disclosed herein, at least one vector disclosed herein, at least one delivery system disclosed herein, or at least one pharmaceutical composition disclosed herein.

The present disclosure also provides a method of treating or preventing a disease or disorder associated with aberrant or increased expression or activity of ACVR1C or a disease or disorder where ACVR1C plays a role in a subject in need thereof, wherein the method comprises administering to the subject an effective amount of at least one isolated oligonucleotide disclosed herein, at least one vector disclosed herein, at least one delivery system disclosed herein, or at least one pharmaceutical composition disclosed herein.

The present disclosure also provides at least one isolated oligonucleotide disclosed herein, a vector of the of the present disclosure encoding at least one isolated oligonucleotide disclosed herein, a delivery system of the present disclosure, or a pharmaceutical composition of the present disclosure, for use in treatment or prevention of a disease or disorder associated with aberrant or increased expression or activity of ACVR1C or a disease or disorder where ACVR1C plays a role, in a subject in need thereof.

The present disclosure also provides use of at least one isolated oligonucleotide disclosed herein, a vector of the of the present disclosure encoding at least one isolated oligonucleotide disclosed herein, a delivery system of the present disclosure, or a pharmaceutical composition of the present disclosure, in the manufacture of a medicament for treatment or prevention of a disease or disorder associated with aberrant or increased expression or activity of ACVR1C or a disease or disorder where ACVR1C plays a role in a subject in need thereof.

Provided herein are methods of inhibiting or downregulating ACVR1C expression or activity in a cell, comprising contacting the cell with the one or more oligonucleotides (e.g., dsRNA or siRNA) targeting ACVR1C as described herein. The one or more oligonucleotides (e.g., dsRNA or siRNA) targeting ACVR1C as described herein can reduce or inhibit ACVR1C activity through the RNAi pathway. The cell can be in vitro, in vivo or ex vivo. For example, the cell can be from a cell line, or in vivo in a subject in need thereof.

In some embodiments, the one or more oligonucleotides (e.g., dsRNA or siRNA) targeting ACVR1C as described herein are capable of inducing RNAi-mediated degradation of a ACVR1C mRNA in a cell of a subject.

As used herein, the terms “contacting,” “introducing” and “administering” are used interchangeably and refer to a process by which dsRNA or siRNA of the present disclosure or a nucleic acid molecule encoding a dsRNA or siRNA of this disclosure is delivered to a cell in order to inhibit or alter or modify expression of a target gene. The dsRNA may be administered in a number of ways including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

In contrast, the term “increase” or grammatical variations thereof as used herein refers to an increase or elevation in the specified level or activity of at least about 5%, about 10%, about 15%, about 25%, about 35%, about 40%, about 50%, about 60%, about 75%, about 80%, about 90%, about 95% or more. Increases in activity can be described in terms of fold change. For example, activity can be increased 1.2×, 1.5×, 2×, 3×, 5×, 6×, 7×, 8×, 9×, 10× or more compared to a baseline level of activity.

As used herein, the term “IC50” or “IC₅₀ value” refers to the concentration of an agent where cell viability is reduced by half The IC₅₀ is thus a measure of the effectiveness of an agent in inhibiting a biological process. In an exemplary model, cell lines are cultured using standard techniques, treated with any of the one or more oligonucleotides (e.g., dsRNA or siRNA) targeting ACVR1C as described herein, and the IC₅₀ value of the oligonucleotides (e.g., dsRNA or siRNA) targeting ACVR1C is calculated after 24, 48 and/or 72 hours to determine its effectiveness in downregulating or inhibiting the level of ACVR1C mRNA or protein to 50%, as compared to the level of ACVR1C mRNA or protein in an untreated cell or in the same cell before initiation of treatment with the isolated oligonucleotide.

Methods of monitoring of ACVR1C mRNA and/or protein expression can be used to characterize gene silencing, and to determine the effectiveness of the compositions described herein. Expression of ACVR1C may be evaluated by any technique known in the art. Examples thereof include immunoprecipitations methods, utilizing ACVR1C antibodies in assays such as ELISAs, western blotting, or immunohistochemistry to visualize ACVR1C protein expression in cells, or flow cytometry. Additional methods include various hybridization methods utilizing a nucleic acid that specifically hybridizes with a nucleic acid encoding ACVR1C or a unique fragment thereof, or a transcription product (e.g., mRNA) or splicing product of said nucleic acid, northern blotting methods, Southern blotting methods, and various PCR-based methods such as RT-PCR, qPCR or digital droplet PCR. ACVR1C mRNA expression may additionally be assessed using high throughput sequencing techniques.

Methods of assaying the effect of individual isolated oligonucleotides (e.g., dsRNA or siRNA) targeting ACVR1C include transfecting representative cell lines with isolated oligonucleotides and measuring viability. For example, cells from representative cell lines can be transfected using methods known in the art, such as the RNAiMAX Lipofectamine kit (Invitrogen) and cultured using any suitable technique known in the art. Optionally additional therapeutic agents as described herein can be added at variable concentrations to cell culture media following transfection. Following a suitable incubation period, such as 24-96 hours, cell viability can be measured using methods such as Cell Titer Glo 2.0 (Promega) to determine cell viability, and/or ACVR1C mRNA and protein levels can be assessed using the methods described herein.

In some embodiments of the methods of inhibiting or downregulating ACVR1C expression or activity in a cell of the present disclosure, the at least one isolated oligonucleotide, the vector, the delivery system, or the pharmaceutical composition is administered parenterally. In some embodiments, the parenteral administration is intravenous, subcutaneous, intraperitoneal, or intramuscular.

In some embodiments of the methods of inhibiting or downregulating ACVR1C expression or activity in a cell of the present disclosure, the method comprises administering the at least one isolated oligonucleotide, the vector, the delivery system, or the pharmaceutical composition, in combination with at least a second therapeutic agent. In some embodiments, the second therapeutic agent is an antibody, a small molecule drug, a peptide, a nucleotide molecule, or a combination thereof. In some embodiments, the second therapeutic agent is an isolated oligonucleotide of the present disclosure.

The present disclosure also provides a method of inhibiting or downregulating the expression or level of ACVR1C in a subject in need thereof, wherein the method comprises administering to the subject an effective amount of a first and at least a second oligonucleotides disclosed herein, wherein the first and at least second oligonucleotides comprise different sequences. In some embodiments, the first and at least second oligonucleotides are administered simultaneously. In some embodiments, the first and at least second oligonucleotides are administered sequentially.

In some embodiments of the methods of inhibiting or downregulating ACVR1C expression or activity in a cell of the present disclosure, the subject is a human. In some embodiments of the methods of inhibiting or downregulating ACVR1C expression or activity in a cell of the present disclosure, the subject experiences symptoms of or suffers from an ACVR1C-associated condition.

In some embodiments of the method of treating or preventing a disease or disorder associated with aberrant or increased expression or activity of ACVR1C or a disease or disorder where ACVR1C plays a role of the present disclosure, the subject is a human. In some embodiments of the method of treating or preventing a disease or disorder associated with aberrant or increased expression or activity of ACVR1C or a disease or disorder where ACVR1C plays a role of the present disclosure, the disease or disorder is an ACVR1C-associated condition.

In some embodiments of the use for treating or preventing a disease or disorder associated with aberrant or increased expression or activity of ACVR1C or a disease or disorder where ACVR1C plays a role of the present disclosure, the subject is a human. In some embodiments of the use for treating or preventing a disease or disorder associated with aberrant or increased expression or activity of ACVR1C or a disease or disorder where ACVR1C plays a role of the present disclosure, the disease or disorder is an ACVR1C-associated condition.

In some embodiments of the use in the manufacture of a medicament for treatment or prevention of a disease or disorder associated with aberrant or increased expression or activity of ACVR1C of the present disclosure, the subject is a human. In some embodiments, the subject is not a human. In some embodiments, the disease or disorder is an ACVR1C-associated condition.

As used herein, the term “ACVR1C-associated condition” is intended to include any condition in which decreasing the expression of ACVR1C (also known as ALK7) is beneficial. Such a condition may be caused, for example, by excessive production of ACVR1C, ACVR1C gene mutations that increase ACVR1C levels or activities. An ACVR1C-associated condition includes but not limited to, obesity, diabetes and metabolic syndromes, cancer, ocular disease (including extraocular retinoblastoma), acquired hyperkeratosis, bone disorders including fibrodysplasia ossificans progressiva and other skeletal abnormalities and any other disease or disorder that is characterized by increased levels of ACVR1C.

### Kits and Articles of Manufacture

The present disclosure also provides kits comprising at least one isolated oligonucleotide disclosed herein, a vector of the present disclosure encoding an isolated oligonucleotide disclosed herein, a delivery system of the present disclosure, or a pharmaceutical composition of the present disclosure. In some embodiments, the vector encodes one isolated oligonucleotide disclosed herein. In some embodiments, the vector encodes more than one isolated oligonucleotide disclosed herein.

The kits are for use in the treatment of diseases related to abnormal or aberrant expression of ACVR1C. The kits are for use in downregulating or inhibiting expression of ACVR1C partially or completely. In some embodiments, the kit is for use in the treatment of disease or downregulating or inhibiting expression of ACVR1C in a mammal. In some embodiments, the mammal is a human, a mouse, a rat, a rabbit, a pig, a bovine, a canine, a feline, an ungulate, an ape, a monkey or an equine species. In some embodiments, the mammal is a human

In some embodiments of the kits of the disclosure, the kit comprises nanoparticles. Nanoparticles comprising the one or more isolated oligonucleotides (e.g., dsRNA or siRNA) targeting ACVR1C mRNA of the present disclosure, can be lyophilized before being packaged in the kit, or can be provided in solution with a pharmaceutically acceptable carrier, diluent of excipient.

In some embodiments of the kits of the disclosure, the kit comprises a therapeutically effective amount of a composition comprising the delivery system of the present disclosure comprising one or more of the isolated oligonucleotides of the present disclosure targeting ACVR1C (dsRNA or siRNA), and instructions for use of the same. In some embodiments, the kit further comprises at least one additional therapeutic agents, as described herein.

Articles of manufacture of the present disclosure include, but are not limited to, instructions for use of the kit in treating diseases related to abnormal or aberrant expression of ACVR1C.

In some embodiments, the kits further comprise instructions for administering the isolated oligonucleotides, the vector, the delivery systems and the pharmaceutical compositions of the disclosure.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed invention. Based on the present disclosure the skilled artisan can identify and employ other components and methodology useful for practicing the present disclosure.

### Oligonucleotide Synthesis

A representative route for the preparation of a conjugate of the present application is described in Scheme A.

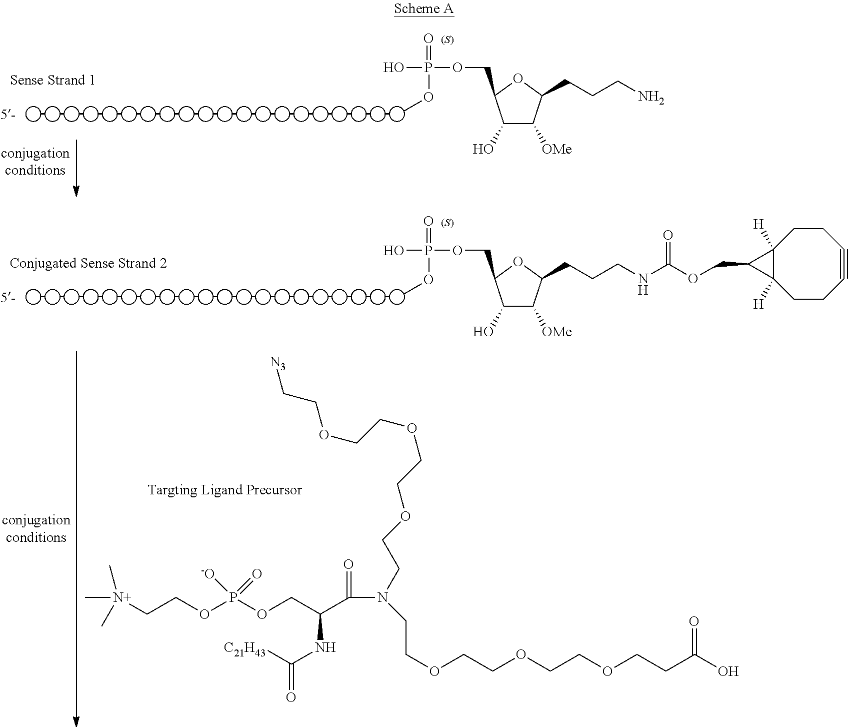

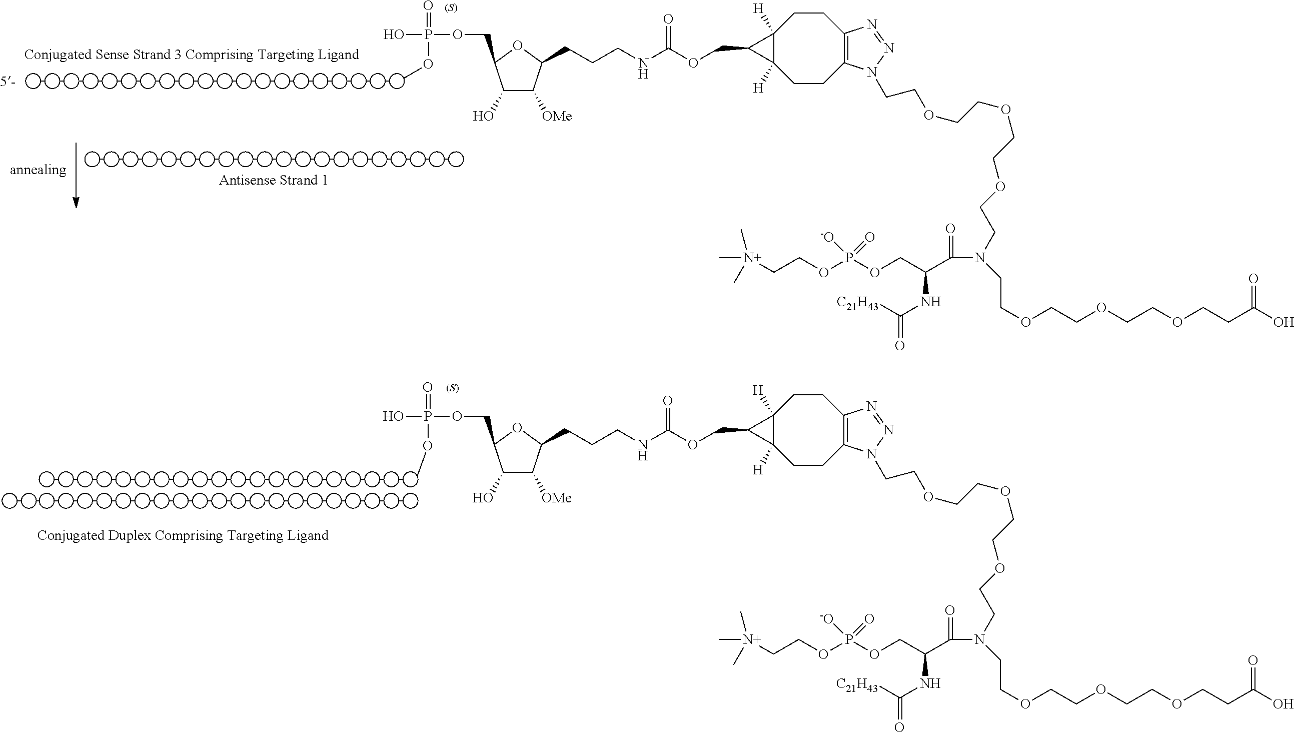

As used herein,

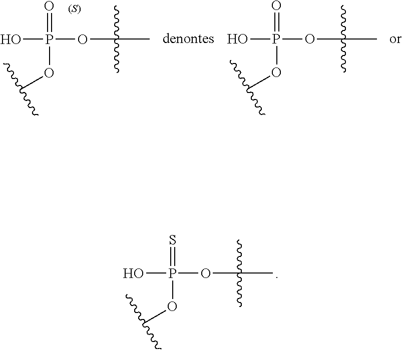

Conjugates of the present disclosure can be prepared by solid-phase synthesis according to standard synthesis protocols or synthesized via post-synthetic conjugations.

For example, oligonucleotide synthesis can be conducted on a solid support to incorporate each nucleoside phosphoramidite, and linker phosphoramidite if present, from 3′-terminus to 5′-terminus to prepare oligo single strands. ETT or BTT can be used as an activator for the coupling reaction. Iodine in water/pyridine/THF can be used to oxidize phosphite-triester (P(III)) to afford phosphate backbones, and DDTT can be used for the preparation of phosphorothioate linkages. Aqueous ammonium can be used to cleave oligos from the solid support and to remove protecting groups globally. The oligo crude can be then concentrated and purified by strong anion exchange or reverse phase HPLC. The purification fractions can be combined and concentrated.

In some cases, oligo single strands can be then conjugated with targeting ligand precursors (e.g., natural or modified lipids) through post-synthetic conjugation to afford conjugates. The conjugation reactions can be performed using standard conjugation methods. The conjugate crude can be further purified by strong anion exchange or reverse phase HPLC. The purification fractions can be combined and concentrated.

The synthesized single strands can be then dialyzed against water using MidiTrap G-25 column or Amicon MWCO filter, concentrated, and their OD amounts can be measured. Based on the equal molar amounts, the sense and antisense strands can be annealed at, e.g., 95° C. for 5 min and cooled down to room temperature to afford the conjugate duplex with, e.g., >90% purity. The solution of the duplex can be lyophilized to afford the desired conjugate, and its amount can be calculated based on the molar amount of single strand used in the annealing.

### EXAMPLES

### Example 1: Oligonucleotide Synthesis

A representative route for the preparation of a conjugate of the present application is described in Scheme A.

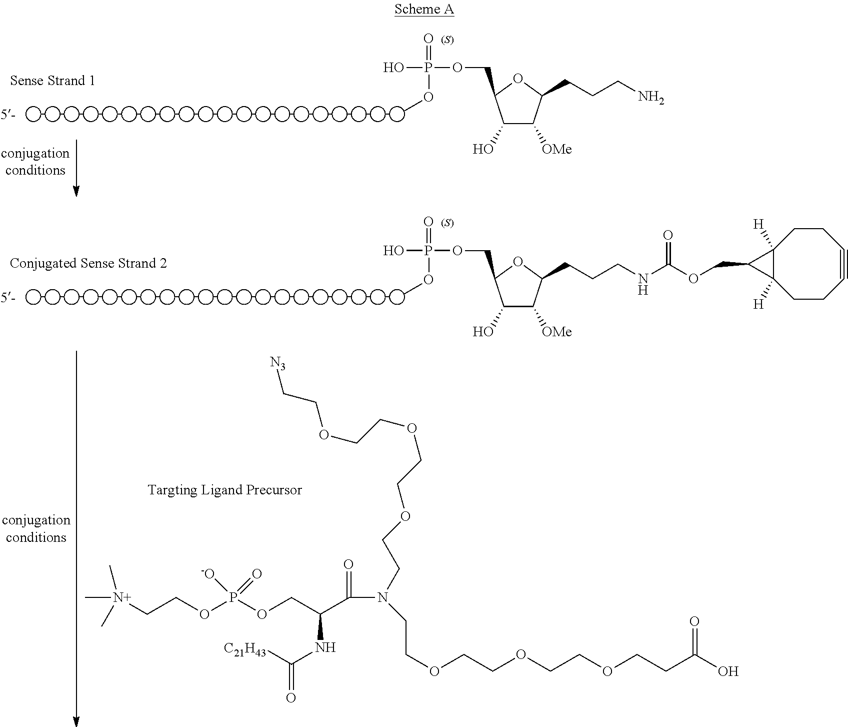

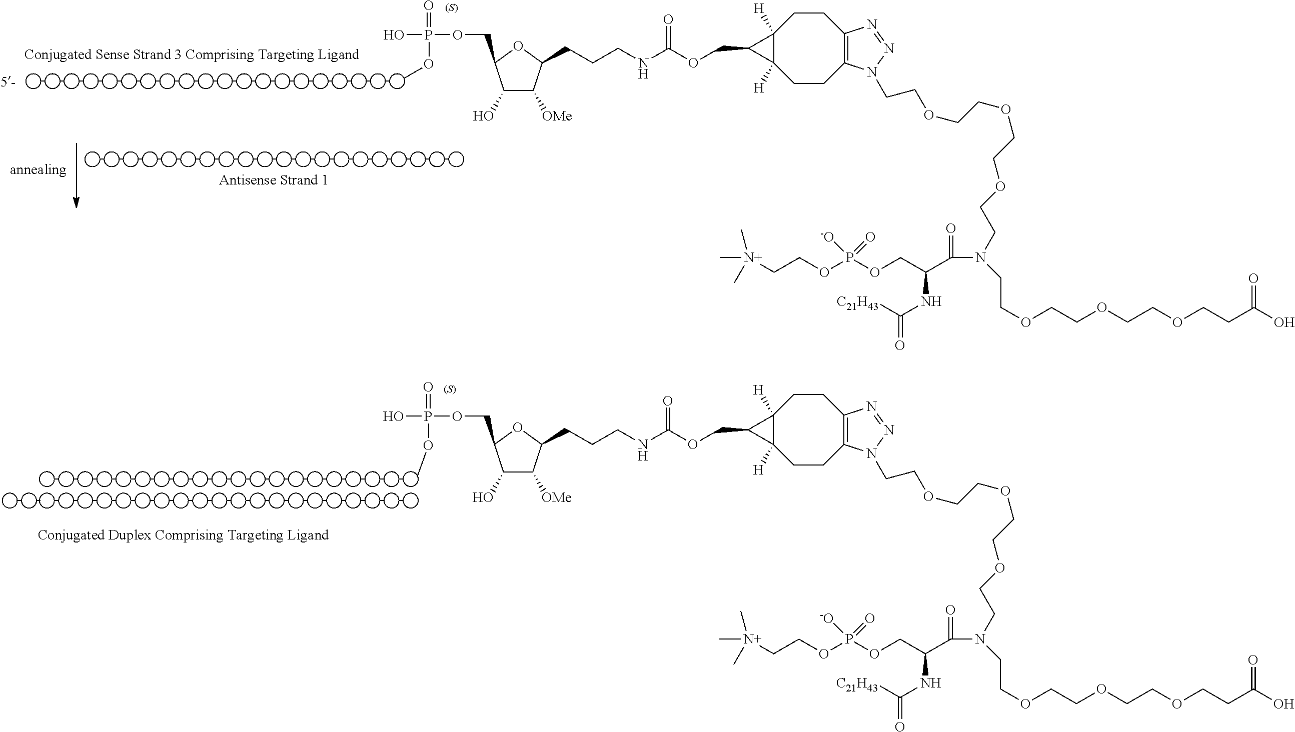

As used herein,

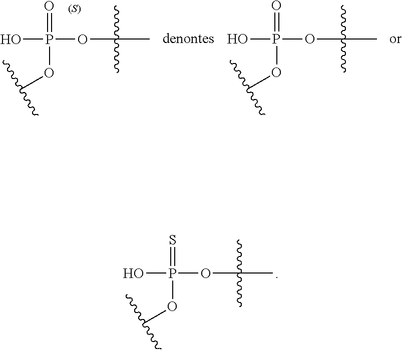

Conjugates of the present disclosure were prepared by solid-phase synthesis according to standard synthesis protocols or synthesized via post-synthetic conjugations.

Briefly, oligonucleotide synthesis were conducted on a solid support to incorporate each nucleoside phosphoramidite, and linker phosphoramidite if present, from 3′-terminus to 5′-terminus to prepare oligo single strands. ETT or BTT was used as an activator for the coupling reaction. Iodine in water/pyridine/THF was used to oxidize phosphite-triester (P(III)) to afford phosphate backbones, and DDTT was used for the preparation of phosphorothioate linkages. Aqueous ammonium was used to cleave oligos from the solid support and to remove protecting groups globally. The oligo crude was then concentrated and purified by strong anion exchange or reverse phase HPLC. The purification fractions were combined and concentrated.

In some cases, oligo single strands were then conjugated with targeting ligand precursors (e.g., natural or modified lipids) through post-synthetic conjugation to afford conjugates. The conjugation reactions were performed using standard conjugation methods. The conjugate crude was further purified by strong anion exchange or reverse phase HPLC. The purification fractions were combined and concentrated.

The synthesized single strands were then dialyzed against water using MidiTrap G-25 column or Amicon MWCO filter, concentrated, and their OD amounts were measured. Based on the equal molar amounts, the sense and antisense strands were annealed at, e.g., 95° C. for 5 min and cooled down to room temperature to afford the conjugate duplex with, e.g., >90% purity. The solution of the duplex was lyophilized to afford the desired conjugate, and its amount was calculated based on the molar amount of single strand used in the annealing.

### Example 2: Post-Synthetic Conjugations of Oligonucleotides

Sense Strand 1 and Antisense Strand 1 were prepared by oligonucleotide solid-phase synthesis as described in Example 1.

Conjugated Sense Strand 2 was synthesized via post-synthetic conjugation. Stock solutions of Sense Strand 1 and reagents were prepared as follows. Oligo Sense Strand 1 was dissolved into water as a 50 mg/mL (7.0 mM) stock. BCN-OSu was dissolved in DMF as a 100 mM stock. DIPEA was dissolved in DMF as a 200 mM stock. 30 mg of oligonucleotide in 600 μL water was diluted with 2.4 mL DMF, followed by DIPEA stock (600 μL, 30 eq) and 420 μL BCN-OSu stock (10 eq). All reagents were pre-cooled to 4° C. before mixing and incubated at room temperature for 30 min. The crude was diluted with saline and dialyzed with water using Amicon Ultra Centrifugal Filters (3KD). The crude was then used directly for the next step without further purifications.

Conjugated Sense Strand 3 Comprising B-1 was synthesized via post-synthetic conjugation. Stock solutions of Conjugated Sense Strand 2 and reagents were prepared as follows. Conjugated Sense Strand 2 was dissolved into water as a 7.4 mg/mL (1.0 mM) solution. Ligand Agent A-1 was dissolved into DMSO as a 5 mg/l mL solution. 1350 μL of Conjugated Sense Strand 2 (10 mg) was mixed with 1 mL of Ligand Agent A-1 in DMSO stock (5 eq) and NMP. The mixture was vortexed at 35° C. for 2 h and diluted with 10 mL water. The reaction mixture was purified by RP-HPLC to afford 4.9 mg of Conjugated Sense Strand 3 Comprising B-1.

Conjugated Duplex Comprising B-1 was annealed based on the equal molar amounts of the sense and antisense strands. Both strands were annealed at 95° C. for 5 min and cooled down to room temperature to afford the conjugate duplex with >90% purity. The solution of the duplex was lyophilized to afford the desired conjugate, and its amount was calculated based on the molar amount of single strand consumed in the annealing.

### Example 3: Design and Testing of siRNA Compounds Against Human ACVR1C mRNA

### Compound Design

A set of 191 siRNAs compounds against human ACVR1C transcript (Accession No: NM_145259.3) were designed (see Table 5 and Table 6).

### Oligonucleotide Synthesis

Oligonucleotides were prepared by solid-phase synthesis according to standard protocols. Briefly, oligonucleotide synthesis was conducted on a solid support to incorporate each nucleoside phosphoramidites from 3′-end to 5′-end to prepare oligo single strands. ETT or BTT was used as an activator for the coupling reaction. Iodine in water/pyridine/THF was used to oxidize phosphite-triester (P(III)) to afford phosphate backbones and DDTT was used for the preparation of phosphorothioate linkages. Aqueous ammonium was used to cleave oligos from solid support and to remove protecting groups globally. The oligonucleotide crude was then concentrated by Genevac and purified by AEX-HPLC. The pure fractions were combined and concentrated, and their purity was analyzed by LC-MS. The oligonucleotides were then dialyzed against water using MidiTrap G-25 column, concentrated, and their OD amounts were measured.

To prepare siRNA duplexes, the sense and antisense strands were annealed at 95° C. for 10 min, based on equal molar amounts, and cooled down to room temperature. The duplex purity was determined by AEX-HPLC, and the solutions were lyophilized to afford the desired siRNA duplex powder.

### In Vitro Screening

The compounds were diluted into the desired concentration with PBS. The psiCheck2-ACVR1C plasmid were diluted into the desired concentration with PBS. The diluted compounds and the psiCheck2-ACVR1C plasmid were then transfected into the cultured Cos7 cells with Lipofectamine 2000 reagents on Day 0. Each compound was tested at two concentrations of 0.4 nM and 0.08 nM. After 24 hours incubation post transfection, Firefly luciferase and Renilla luciferase expression in cell lysis was detected by Dual Glo Luciferase Reporter Gene Assay Kit. Data is presented as % of ACVR1C mRNA remaining relative to mock transfection when normalized to Firefly luciferase expression levels (Mean, +/−SD).

The percentage of human ACVR1C mRNA remaining in cells relative to mock transfection when normalized to Firefly luciferase expression levels, was determined for each compound at a concentration of either 0.4 nM or 0.08 nM. The results identified several compounds that were able to reduce the level of human ACVR1C mRNA in transfected cells by 20% to 50% or more than 50% at the defined concentrations as described in Table 7 (see, columns titled “% mRNA remaining at 0.4 nM” and “% mRNA remaining at 0.08 nM”).

### In Vivo HDI Screening

6- to 8-week-old female BALB/c mice were administered subcutaneously with a single dose of 1 mg/kg of selected siRNA compounds for one dose screening (Table 7, see column titled “% mRNA remaining at 1 mg/kg, single dose”), or with a single dose of 0.5, 1.0, or 2 mg/kg of selected siRNA compounds for dose-response study (Table 7, see columns titled “% mRNA remaining at 0.5 mg/kg,” “% mRNA remaining at 1 mg/kg,” and “% mRNA remaining at 2 mg/kg”). Three days later, animals were transiently transfected in vivo with ACVR1C-expressing plasmid by hydrodynamic tail vein injection (HDI). 24 hours post-HDI administration, animals were sacrificed, and liver tissues were isolated and stored in RNA later at 4° C. RNA in the liver was extracted using MNTR/FX96 (LR) kit (GeneOn BioTech). cDNA was generated from the isolated RNA using Takara-PrimeScript™ RT Reagent Kit with gDNA Eraser and a TaqMan RT-qPCR gene expression assay was conducted to analyze the compound potency in silencing ACVR1C mRNA. Data is presented as % reduction of ACVR1C mRNA relative to mock transfection when normalized to NeoR mRNA levels (Mean, +/−SD). The results of the 1 mg/kg single dose in vivo potency experiments are shown in Table 7 (see column titled “% mRNA remaining at 1 mg/kg, single dose”). The results of the in vivo dose response experiments are shown in Table 7 (see columns titled “% mRNA remaining at 0.5 mg/kg,” “% mRNA remaining at 1 mg/kg,” and “% mRNA remaining at 2 mg/kg”).

Additional screening was carried out for selected siRNA compounds with a single dose of 0.056 mg/kg, 0.167 mg/kg, or 0.5 mg/kg as described above. The results of the in vivo dose response experiments are shown in Table 7 (see columns titled “% mRNA remaining at 0.056 mg/kg,” “% mRNA remaining at 0.167 mg/kg,” and “% mRNA remaining at 0.5 mg/kg.”).

The tested compounds were able to reduce the level of human ACVR1C mRNA.

### Example 4: In Vivo Potency Evaluation in Non-Human Primates (NHPs)

Selected sequences were further evaluated for in vivo efficacy in non-human primates (NHPs). On Day 0, groups of 3-4 cynomolgus monkeys (Macaca fascicularis) received a single subcutaneous dose of 3 mg/kg Duplex 537, 538, 539, 540, or 541, or PBS vehicle control. Subcutaneous adipose biopsies were collected on Day −14 (pre-dose; Week −2) and on Days 14, 28, 56, and 84 (Weeks 2, 4, 8, and 12) post-dose. Total RNA was extracted from the biopsy samples and ACVR1C mRNA levels were quantified by RT-qPCR, with ARL1 used as the housekeeping gene for normalization.

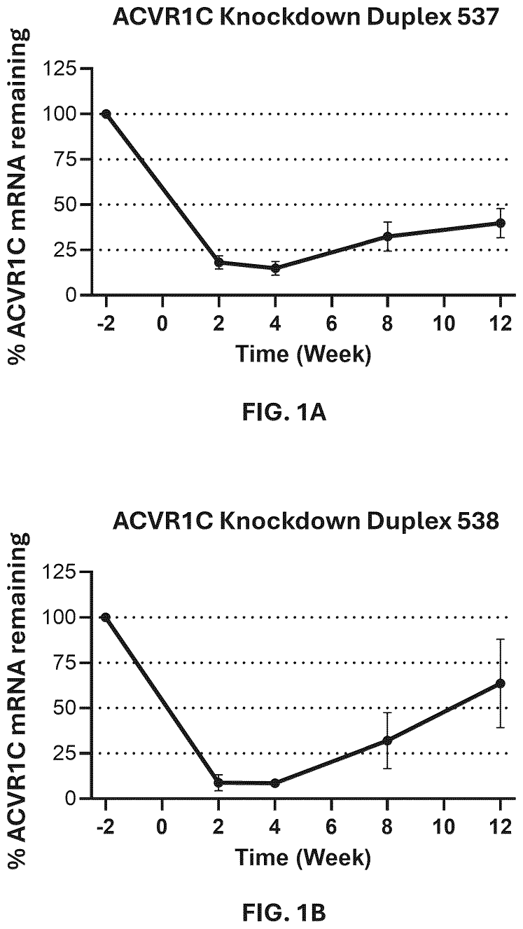

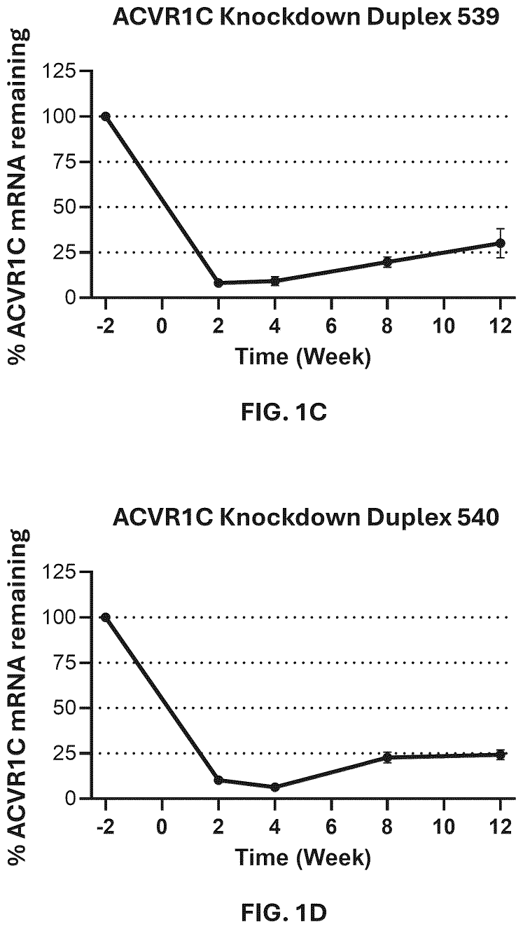

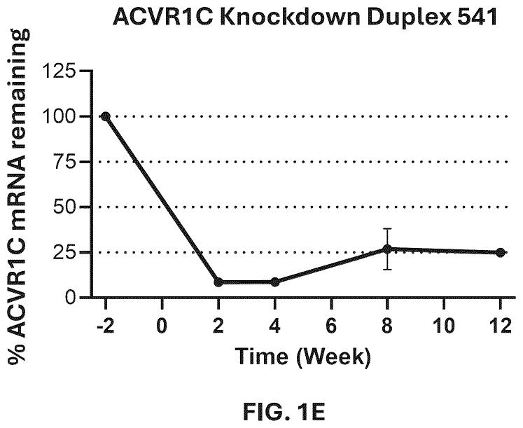

Measurements of ACVR1C mRNA levels are shown in FIGS. 1A-1E and recorded in Table 8. All tested duplexes reduced ACVR1C mRNA levels under 25% by Week 2 post-dose.

Additional sequences were further evaluated for in vivo efficacy in NHPs. On Day 0, groups of 2-3 cynomolgus monkeys (Macaca fascicularis) each received a single subcutaneous (SC) administration of 0.75 mg/kg Duplex 540 and 542, or PBS as a vehicle control. Subcutaneous adipose tissue biopsies were collected on Day −14 (pre-dose) and on Days 14, 28, 56, and 84 following dosing. Total RNA was extracted from the biopsy samples, and ACVR1C mRNA levels were quantified by RT-qPCR. Expression levels were normalized using ARL1 as a housekeeping gene.

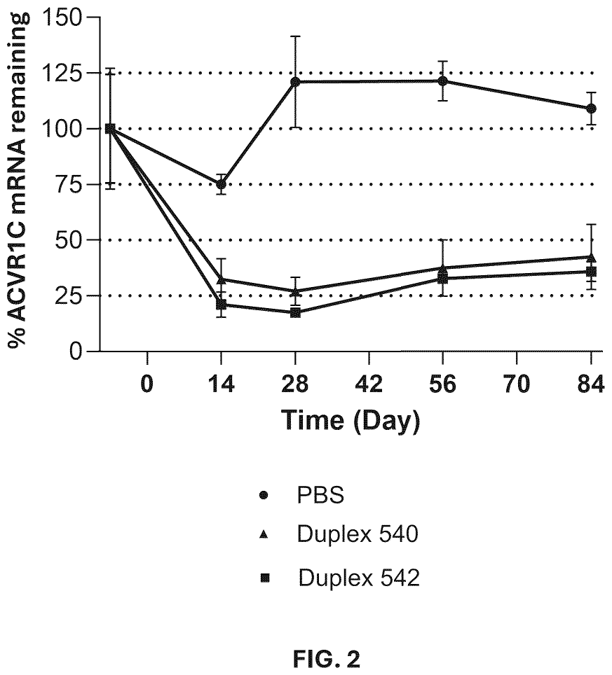

Measurements of ACVR1C mRNA levels are shown in FIG. 2 and recorded in Table 9. All tested duplexes reduced ACVR1C mRNA levels to about 25% by Week 2 post-dose.

Sequences of the Disclosure

Note: “mA” or “[mA]” refers to 2′-O-methyl-adenosine; “mU” or “[mU]” refers to 2-O-methyl-uridine; “mG” or “[mG]” refers to 2′-O-methyl-guanosine; “mC” or “[mC]” refers to 2′-O-methyl-cytidine; “fA” or “[fA]” refers to 2′-fluoro-adenosine; “fU” or “[fU]” refers to 2′-fluoro-uridine; “fG” or “[fG]” refers to 2′-fluoro-guanosine; “fC” or “[fC]” refers to 2′-fluoro-cytidine; “s” (e.g., in “mAs,”“fAs,” etc.) refers to phosphorothioate internucleotide linkage; “G1bG1bG1b” or “[G1b][G1b][G1b]” refers to 3 iterations of (2R,3R,4R,5S)-5-(3-(5-(((2R,3R,4R,5R,6R) - 3-acetamido-4,5-dihydroxy-6-(hydroxymethyl)tetrahydro-2H-pyran-2-yl)oxy) pentanamido)propyl)-2-((hydroxyl)methyl)-4-methoxytetrahydrofuran-3-oxyl; “G1b-BCN-C22PC2” or “[G1b-BCN-C22PC2]” refers to (S)-12-(6-carboxyhexyl)-14-docosanamido-1-((5aS,6R,6aR)-6-((((3-((2S,3R,4R,5R)-4-hydroxy-3-methoxy-5-(oxylmethyl)tetrahydrofuran-2-yl) propyl)carbamoyl)oxy)methyl)-5,5a,6,6a, 7,8-hexahydrocyclopropa[5,6]cycloocta[1,2-d][1,2,3]triazol-1(4H)-yl)-13-oxo-3,6,9-trioxa-12-azapentadecan-15-yl (2-(trimethylammonio)ethyl) phosphate; and “C-EPmU” (e.g., in “C-EPmUs”) refers to 3-(2,4-dioxo -3,4-dihydropyrimidin-1(2H)-yl)-2-methoxy-5-(2-phosphonoethyl)cyclopentan-1-oxyl.

TABLE 7
In Vitro and In Vivo Potency of Exemplary
Sequences of the Disclosure

| | | | | | % mRNA | |
|---|---|---|---|---|---|---|
| | | | | | remaining | % mRNA |
| | | | | | at 1 | remaining |
| | % mRNA | | % mRNA | | mg/kg, | at |
| | remaining | | remaining | | single | 0.5 |
| Duplex | at 0.4 nM | | at 0.08 nM | | dose | mg/kg |
| No. | Mean | SD | Mean | SD | Mean | Mean |
| 269 | 56.56 | 0.30 | 73.33 | 1.66 | — | — |
| 270 | 39.39 | 1.44 | 63.49 | 1.26 | 25.89 | 32.52 |
| 271 | 39.03 | 5.16 | 63.27 | 1.58 | — | — |
| 272 | 47.15 | 1.22 | 72.13 | 3.02 | — | — |
| 273 | 46.04 | 4.18 | 67.29 | 4.48 | — | — |
| 274 | 41.16 | 3.89 | 59.19 | 2.66 | — | — |
| 275 | 38.56 | 5.32 | 53.55 | 4.15 | 27.99 | 33.33 |
| 276 | 43.23 | 3.87 | 61.46 | 2.98 | — | — |
| 277 | 34.67 | 3.94 | 48.86 | 4.40 | — | — |
| 278 | 42.76 | 2.19 | 69.34 | 6.00 | — | — |
| 279 | 36.40 | 3.87 | 54.61 | 2.91 | — | — |
| 280 | 36.93 | 3.17 | 54.14 | 1.58 | — | — |
| 281 | 31.24 | 4.64 | 44.70 | 2.46 | 32.45 | — |
| 282 | 50.93 | 5.32 | 70.77 | 4.79 | — | — |
| 283 | 39.71 | 1.30 | 56.60 | 4.32 | — | — |
| 284 | 36.35 | 1.67 | 54.81 | 2.54 | — | — |
| 285 | 51.69 | 0.86 | 75.00 | 1.32 | — | — |
| 286 | 46.72 | 2.39 | 70.99 | 2.97 | — | — |
| 287 | 55.29 | 5.83 | 76.30 | 3.84 | — | — |
| 288 | 31.19 | 0.68 | 48.73 | 3.37 | 41.33 | — |
| 289 | 33.30 | 0.31 | 53.83 | 1.85 | — | — |
| 290 | 60.08 | 3.95 | 83.02 | 1.91 | — | — |
| 291 | 37.38 | 0.47 | 58.76 | 3.86 | — | — |
| 292 | 48.99 | 1.05 | 72.42 | 2.13 | — | — |
| 293 | 42.08 | 2.90 | 71.17 | 4.65 | — | — |
| 294 | 39.12 | 3.23 | 59.69 | 1.53 | — | — |
| 295 | 39.20 | 6.21 | 63.70 | 2.53 | — | — |
| 296 | 40.10 | 4.65 | 57.95 | 4.31 | — | — |
| 297 | 43.92 | 2.75 | 67.39 | 2.92 | — | — |
| 298 | 63.70 | 7.59 | 86.64 | 5.88 | — | — |
| 299 | 42.82 | 4.41 | 51.93 | 1.65 | — | — |
| 300 | 46.60 | 5.96 | 68.70 | 2.93 | — | — |
| 301 | 46.04 | 5.20 | 68.60 | 3.44 | — | — |
| 302 | 42.98 | 2.57 | 58.75 | 5.16 | 29.77 | 38.74 |
| 303 | 50.01 | 1.54 | 62.06 | 3.03 | 26.43 | 32.11 |
| 304 | 78.42 | 1.59 | 101.91 | 2.42 | — | — |
| 305 | 55.48 | 4.63 | 76.90 | 4.50 | — | — |
| 306 | 59.71 | 5.20 | 78.82 | 5.36 | — | — |
| 307 | 59.34 | 5.54 | 88.51 | 1.26 | — | — |
| 308 | 61.65 | 3.94 | 78.80 | 8.05 | — | — |
| 309 | 49.43 | 4.34 | 62.68 | 4.40 | — | — |
| 310 | 49.64 | 2.19 | 69.06 | 2.20 | — | — |
| 311 | 51.82 | 4.47 | 70.55 | 1.82 | — | — |
| 312 | 61.45 | 1.25 | 84.55 | 2.54 | — | — |
| 313 | 56.42 | 7.54 | 80.99 | 6.99 | — | — |
| 314 | 47.66 | 3.52 | 71.76 | 4.88 | — | — |
| 315 | 41.09 | 1.94 | 61.45 | 5.55 | — | — |
| 316 | 55.74 | 2.33 | 79.85 | 6.44 | — | — |
| 317 | 65.19 | 3.95 | 68.47 | 0.58 | — | — |
| 318 | 75.92 | 4.07 | 79.52 | 6.97 | — | — |
| 319 | 71.50 | 4.38 | 83.93 | 2.31 | — | — |
| 320 | 67.75 | 1.88 | 82.33 | 2.53 | — | — |
| 321 | 76.97 | 4.11 | 95.27 | 2.77 | — | — |
| 322 | 81.60 | 2.90 | 93.12 | 4.04 | — | — |
| 323 | 29.06 | 2.00 | 39.04 | 3.12 | 38.54 | — |
| 324 | 34.27 | 4.40 | 54.09 | 2.08 | — | — |
| 325 | 24.30 | 2.46 | 38.89 | 3.51 | 21.68 | 38.12 |
| 326 | 54.53 | 3.96 | 78.89 | 4.87 | — | — |
| 327 | 48.85 | 7.03 | 77.46 | 7.86 | — | — |
| 328 | 30.89 | 3.61 | 50.01 | 2.15 | 40.50 | — |
| 329 | 25.33 | 1.11 | 46.82 | 1.99 | 33.61 | — |
| 330 | 76.75 | 0.53 | 95.56 | 2.33 | — | — |
| 331 | 70.04 | 6.37 | 89.95 | 4.75 | — | — |
| 332 | 51.19 | 2.97 | 75.16 | 3.92 | — | — |
| 333 | 62.85 | 3.41 | 82.85 | 6.21 | — | — |
| 334 | 45.18 | 2.25 | 56.57 | 7.95 | — | — |
| 335 | 53.58 | 3.67 | 74.49 | 1.70 | — | — |
| 336 | 49.03 | 0.35 | 69.11 | 7.43 | — | — |
| 337 | 92.26 | 4.98 | 96.72 | 7.75 | — | — |
| 338 | 47.93 | 4.34 | 68.48 | 2.02 | — | — |
| 339 | 89.15 | 8.62 | 100.64 | 3.96 | — | — |
| 340 | 83.74 | 4.76 | 100.41 | 3.32 | — | — |
| 341 | 64.98 | 1.72 | 82.61 | 4.96 | — | — |
| 342 | 87.75 | 2.17 | 103.79 | 10.06 | — | — |
| 343 | 88.01 | 10.34 | 104.94 | 3.33 | — | — |
| 344 | 77.71 | 10.12 | 96.39 | 22.17 | — | — |
| 345 | 71.70 | 5.35 | 98.34 | 11.44 | — | — |
| 346 | 69.59 | 5.37 | 112.74 | 13.94 | — | — |
| 347 | 82.74 | 19.86 | 107.09 | 10.71 | — | — |
| 348 | 68.66 | 3.95 | 88.29 | 6.06 | — | — |
| 349 | 46.90 | 12.26 | 64.04 | 8.74 | — | — |
| 350 | 43.90 | 1.06 | 63.43 | 6.52 | — | — |
| 351 | 52.76 | 8.65 | 74.08 | 5.78 | — | — |
| 352 | 62.05 | 12.58 | 85.03 | 8.12 | — | — |
| 353 | 57.60 | 3.87 | 101.82 | 18.50 | — | — |
| 354 | 48.01 | 6.24 | 67.23 | 10.49 | — | — |
| 355 | 74.70 | 12.81 | 96.13 | 12.98 | — | — |
| 356 | 45.13 | 0.60 | 61.33 | 9.91 | — | — |
| 357 | 63.95 | 3.93 | 83.25 | 22.37 | — | — |
| 358 | 41.43 | 3.65 | 57.68 | 6.91 | — | — |
| 359 | 38.63 | 5.27 | 51.41 | 3.07 | 27.89 | 34.22 |
| 360 | 38.21 | 1.86 | 55.75 | 1.38 | — | — |
| 361 | 38.49 | 4.05 | 47.32 | 7.44 | 33.68 | — |
| 362 | 38.05 | 9.18 | 58.60 | 8.84 | 23.08 | 55.43 |
| 363 | 55.03 | 3.38 | 70.90 | 2.49 | — | — |
| 364 | 45.75 | 10.55 | 59.66 | 11.41 | — | — |
| 365 | 60.43 | 5.96 | 86.77 | 9.71 | — | — |
| 366 | 55.28 | 5.67 | 76.03 | 12.98 | — | — |
| 367 | 46.57 | 4.38 | 59.26 | 5.35 | — | — |
| 368 | 47.80 | 3.93 | 64.99 | 14.32 | — | — |
| 369 | 60.81 | 7.96 | 77.28 | 4.09 | — | — |
| 370 | 61.09 | 5.55 | 89.41 | 10.75 | — | — |
| 371 | 53.80 | 5.66 | 63.99 | 1.76 | — | — |
| 372 | 59.71 | 9.70 | 76.23 | 12.25 | — | — |
| 373 | 58.19 | 6.78 | 76.26 | 10.79 | — | — |
| 374 | 56.86 | 3.38 | 72.55 | 11.91 | — | — |
| 375 | 36.74 | 2.01 | 59.21 | 6.47 | — | — |
| 376 | 64.49 | 2.20 | 67.91 | 3.33 | — | — |
| 377 | 59.01 | 7.73 | 68.90 | 7.55 | — | — |
| 378 | 64.30 | 9.87 | 86.89 | 13.76 | — | — |
| 379 | 60.73 | 15.22 | 77.86 | 4.17 | — | — |
| 380 | 55.92 | 9.09 | 80.94 | 8.09 | — | — |
| 381 | 71.37 | 11.28 | 102.58 | 9.61 | — | — |
| 382 | 40.26 | 2.70 | 59.82 | 13.44 | 23.23 | 43.40 |
| 383 | 45.12 | 9.52 | 91.94 | 13.50 | — | — |
| 384 | 63.11 | 7.02 | 79.65 | 5.92 | — | — |
| 385 | 94.27 | 13.43 | 100.38 | 3.60 | — | — |
| 386 | 63.14 | 4.87 | 80.10 | 7.02 | — | — |
| 387 | 68.81 | 10.47 | 86.60 | 5.26 | — | — |
| 388 | 63.45 | 2.00 | 69.46 | 5.98 | — | — |
| 389 | 44.49 | 4.08 | 53.35 | 0.45 | — | — |
| 390 | 51.38 | 5.96 | 67.44 | 4.41 | — | — |
| 391 | 60.22 | 2.59 | 69.63 | 3.88 | — | — |
| 392 | 51.43 | 6.40 | 69.09 | 13.40 | — | — |
| 393 | 59.10 | 4.29 | 77.93 | 11.56 | — | — |
| 394 | 58.40 | 10.22 | 62.64 | 5.54 | — | — |
| 395 | 66.54 | 10.55 | 70.32 | 10.32 | — | — |
| 396 | 56.84 | 11.97 | 82.21 | 6.45 | — | — |
| 397 | 60.28 | 7.00 | 53.92 | 8.86 | — | — |
| 398 | 66.82 | 1.24 | 78.05 | 1.45 | — | — |
| 399 | 61.57 | 4.71 | 63.67 | 4.44 | — | — |
| 400 | 62.13 | 3.88 | 60.36 | 0.85 | — | — |
| 401 | 56.41 | 2.64 | 75.69 | 8.73 | — | — |
| 402 | 62.91 | 5.93 | 77.42 | 9.00 | — | — |
| 403 | 84.95 | 2.88 | 111.23 | 5.25 | — | — |
| 404 | 65.62 | 4.82 | 99.83 | 8.71 | — | — |
| 405 | 71.63 | 3.99 | 96.26 | 1.25 | — | — |
| 406 | 81.15 | 7.26 | 92.09 | 7.03 | — | — |
| 407 | 75.45 | 3.75 | 79.80 | 4.26 | — | — |
| 408 | 78.05 | 3.91 | 84.00 | 2.81 | — | — |
| 409 | 73.08 | 5.96 | 68.23 | 4.56 | — | — |
| 410 | 93.79 | 3.92 | 102.47 | 6.21 | — | — |
| 411 | 85.10 | 3.10 | 85.75 | 6.87 | — | — |
| 412 | 71.36 | 6.94 | 87.12 | 10.39 | — | — |
| 413 | 74.89 | 5.96 | 103.56 | 20.56 | — | — |
| 414 | 77.23 | 14.63 | 97.32 | 9.31 | — | — |
| 415 | 76.86 | 9.35 | 93.33 | 2.59 | — | — |
| 416 | 103.94 | 5.08 | 89.49 | 7.69 | — | — |
| 417 | 98.19 | 4.21 | 104.69 | 7.07 | — | — |
| 418 | 108.90 | 10.08 | 107.01 | 14.06 | — | — |
| 419 | 65.55 | 12.49 | 79.76 | 5.36 | — | — |
| 420 | 81.54 | 4.74 | 83.53 | 11.67 | — | — |
| 421 | 70.72 | 11.28 | 106.33 | 35.95 | — | — |
| 422 | 78.70 | 9.78 | 84.54 | 6.45 | — | — |
| 423 | 79.87 | 7.22 | 92.46 | 8.22 | — | — |
| 424 | 72.38 | 6.00 | 78.99 | 8.72 | — | — |
| 425 | 81.94 | 5.46 | 84.94 | 5.75 | — | — |
| 426 | 91.59 | 12.91 | 103.21 | 4.17 | — | — |
| 427 | 88.89 | 6.22 | 89.60 | 19.46 | — | — |
| 428 | 61.10 | 11.67 | 81.42 | 1.01 | — | — |
| 429 | 67.55 | 9.92 | 88.80 | 10.19 | — | — |
| 430 | 90.09 | 16.26 | 90.26 | 18.71 | — | — |
| 431 | 90.91 | 13.33 | 100.84 | 21.55 | — | — |
| 432 | 73.54 | 3.36 | 89.09 | 9.08 | — | — |
| 433 | 106.51 | 11.68 | 98.31 | 11.01 | — | — |
| 434 | 83.27 | 6.19 | 90.90 | 3.97 | — | — |
| 435 | 77.73 | 2.15 | 80.02 | 7.08 | — | — |
| 436 | 72.72 | 6.93 | 82.79 | 14.00 | — | — |
| 437 | 94.99 | 17.22 | 98.69 | 7.58 | — | — |
| 438 | 85.74 | 5.75 | 91.06 | 9.92 | — | — |
| 439 | 74.90 | 6.48 | 78.11 | 9.32 | — | — |
| 440 | 85.24 | 8.46 | 93.37 | 4.80 | — | — |
| 441 | 87.55 | 6.03 | 93.27 | 14.48 | — | — |
| 442 | 80.82 | 3.43 | 89.30 | 12.53 | — | — |
| 443 | 73.34 | 3.92 | 84.03 | 2.57 | — | — |
| 444 | 78.89 | 6.56 | 79.14 | 5.88 | — | — |
| 445 | 63.32 | 2.25 | 81.93 | 7.47 | — | — |
| 446 | 71.48 | 4.09 | 88.35 | 3.06 | — | — |
| 447 | 83.70 | 5.27 | 71.24 | 5.14 | — | — |
| 448 | 73.30 | 2.01 | 76.55 | 5.62 | — | — |
| 449 | 59.54 | 11.71 | 78.42 | 6.00 | — | — |
| 450 | 68.53 | 8.39 | 81.15 | 1.66 | — | — |
| 451 | 74.70 | 4.35 | 77.38 | 7.04 | — | — |
| 452 | 85.84 | 4.47 | 107.11 | 11.33 | — | — |
| 453 | 76.13 | 6.21 | 94.85 | 17.27 | — | — |
| 454 | 67.71 | 1.57 | 70.68 | 3.84 | — | — |
| 455 | 66.33 | 5.48 | 80.89 | 6.26 | — | — |
| 456 | 60.05 | 5.56 | 70.34 | 13.05 | — | — |
| 457 | 77.92 | 5.34 | 72.18 | 1.03 | — | — |

| | | | % mRNA | % mRNA | % mRNA |
|---|---|---|---|---|---|
| | % mRNA | % mRNA | remaining | remaining | remaining |
| | remaining | remaining | at 0.056 | at 0.167 | at 0.5 |
| Duplex | at 1 mg/kg | at 2 mg/kg | mg/kg | mg/kg | mg/kg |
| No. | Mean | Mean | Mean | Mean | Mean |
| 269 | — | — | — | — | — |
| 270 | 32.72 | 35.90 | 62.35 | 41.22 | 33.01 |
| 271 | — | — | — | — | — |
| 272 | — | — | — | — | — |
| 273 | — | — | — | — | — |
| 274 | — | — | — | — | — |
| 275 | 38.38 | 28.94 | — | — | — |
| 276 | — | — | — | — | — |
| 277 | — | — | — | — | — |
| 278 | — | — | — | — | — |
| 279 | — | — | — | — | — |
| 280 | — | — | — | — | — |
| 281 | — | — | 61.39 | 52.62 | 60.17 |
| 282 | — | — | — | — | — |
| 283 | — | — | — | — | — |
| 284 | — | — | — | — | — |
| 285 | — | — | — | — | — |
| 286 | — | — | — | — | — |
| 287 | — | — | — | — | — |
| 288 | — | — | 65.35 | 42.65 | 26.03 |
| 289 | — | — | — | — | — |
| 290 | — | — | — | — | — |
| 291 | — | — | — | — | — |
| 292 | — | — | — | — | — |
| 293 | — | — | — | — | — |
| 294 | — | — | — | — | — |
| 295 | — | — | — | — | — |
| 296 | — | — | — | — | — |
| 297 | — | — | — | — | — |
| 298 | — | — | — | — | — |
| 299 | — | — | — | — | — |
| 300 | — | — | — | — | — |
| 301 | — | — | — | — | — |
| 302 | 32.39 | 28.14 | 57.13 | 54.86 | 33.44 |
| 303 | 31.20 | 42.16 | 91.27 | 48.11 | 35.20 |
| 304 | — | — | — | — | — |
| 305 | — | — | — | — | — |
| 306 | — | — | — | — | — |
| 307 | — | — | — | — | — |
| 308 | — | — | — | — | — |
| 309 | — | — | — | — | — |
| 310 | — | — | — | — | — |
| 311 | — | — | — | — | — |
| 312 | — | — | — | — | — |
| 313 | — | — | — | — | — |
| 314 | — | — | — | — | — |
| 315 | — | — | — | — | — |
| 316 | — | — | — | — | — |
| 317 | — | — | — | — | — |
| 318 | — | — | — | — | — |
| 319 | — | — | — | — | — |
| 320 | — | — | — | — | — |
| 321 | — | — | — | — | — |
| 322 | — | — | — | — | — |
| 323 | — | — | 73.23 | 88.19 | 47.96 |
| 324 | — | — | — | — | — |
| 325 | 23.84 | 28.23 | 63.45 | 46.95 | 41.95 |
| 326 | — | — | — | — | — |
| 327 | — | — | — | — | — |
| 328 | — | — | 60.50 | 46.55 | 30.51 |
| 329 | — | — | 85.95 | 40.50 | 51.10 |
| 330 | — | — | — | — | — |
| 331 | — | — | — | — | — |
| 332 | — | — | — | — | — |
| 333 | — | — | — | — | — |
| 334 | — | — | — | — | — |
| 335 | — | — | — | — | — |
| 336 | — | — | — | — | — |
| 337 | — | — | — | — | — |
| 338 | — | — | — | — | — |
| 339 | — | — | — | — | — |
| 340 | — | — | — | — | — |
| 341 | — | — | — | — | — |
| 342 | — | — | — | — | — |
| 343 | — | — | — | — | — |
| 344 | — | — | — | — | — |
| 345 | — | — | — | — | — |
| 346 | — | — | — | — | — |
| 347 | — | — | — | — | — |
| 348 | — | — | — | — | — |
| 349 | — | — | — | — | — |
| 350 | — | — | — | — | — |
| 351 | — | — | — | — | — |
| 352 | — | — | — | — | — |
| 353 | — | — | — | — | — |
| 354 | — | — | — | — | — |
| 355 | — | — | — | — | — |
| 356 | — | — | — | — | — |
| 357 | — | — | — | — | — |
| 358 | — | — | — | — | — |
| 359 | 50.15 | 32.16 | — | — | — |
| 360 | — | — | — | — | — |
| 361 | — | — | — | — | — |
| 362 | 30.99 | 31.54 | 65.51 | 41.66 | 45.74 |
| 363 | — | — | — | — | — |
| 364 | — | — | — | — | — |
| 365 | — | — | — | — | — |
| 366 | — | — | — | — | — |
| 367 | — | — | — | — | — |
| 368 | — | — | — | — | — |
| 369 | — | — | — | — | — |
| 370 | — | — | — | — | — |
| 371 | — | — | — | — | — |
| 372 | — | — | — | — | — |
| 373 | — | — | — | — | — |
| 374 | — | — | — | — | — |
| 375 | — | — | — | — | — |
| 376 | — | — | — | — | — |
| 377 | — | — | — | — | — |
| 378 | — | — | — | — | — |
| 379 | — | — | — | — | — |
| 380 | — | — | — | — | — |
| 381 | — | — | — | — | — |
| 382 | 44.06 | 20.86 | — | — | — |
| 383 | — | — | — | — | — |
| 384 | — | — | — | — | — |
| 385 | — | — | — | — | — |
| 386 | — | — | — | — | — |
| 387 | — | — | — | — | — |
| 388 | — | — | — | — | — |
| 389 | — | — | — | — | — |
| 390 | — | — | — | — | — |
| 391 | — | — | — | — | — |
| 392 | — | — | — | — | — |
| 393 | — | — | — | — | — |
| 394 | — | — | — | — | — |
| 395 | — | — | — | — | — |
| 396 | — | — | — | — | — |
| 397 | — | — | — | — | — |
| 398 | — | — | — | — | — |
| 399 | — | — | — | — | — |
| 400 | — | — | — | — | — |
| 401 | — | — | — | — | — |
| 402 | — | — | — | — | — |
| 403 | — | — | — | — | — |
| 404 | — | — | — | — | — |
| 405 | — | — | — | — | — |
| 406 | — | — | — | — | — |
| 407 | — | — | — | — | — |
| 408 | — | — | — | — | — |
| 409 | — | — | — | — | — |
| 410 | — | — | — | — | — |
| 411 | — | — | — | — | — |
| 412 | — | — | — | — | — |
| 413 | — | — | — | — | — |
| 414 | — | — | — | — | — |
| 415 | — | — | — | — | — |
| 416 | — | — | — | — | — |
| 417 | — | — | — | — | — |
| 418 | — | — | — | — | — |
| 419 | — | — | — | — | — |
| 420 | — | — | — | — | — |
| 421 | — | — | — | — | — |
| 422 | — | — | — | — | — |
| 423 | — | — | — | — | — |
| 424 | — | — | — | — | — |
| 425 | — | — | — | — | — |
| 426 | — | — | — | — | — |
| 427 | — | — | — | — | — |
| 428 | — | — | — | — | — |
| 429 | — | — | — | — | — |
| 430 | — | — | — | — | — |
| 431 | — | — | — | — | — |
| 432 | — | — | — | — | — |
| 433 | — | — | — | — | — |
| 434 | — | — | — | — | — |
| 435 | — | — | — | — | — |
| 436 | — | — | — | — | — |
| 437 | — | — | — | — | — |
| 438 | — | — | — | — | — |
| 439 | — | — | — | — | — |
| 440 | — | — | — | — | — |
| 441 | — | — | — | — | — |
| 442 | — | — | — | — | — |
| 443 | — | — | — | — | — |
| 444 | — | — | — | — | — |
| 445 | — | — | — | — | — |
| 446 | — | — | — | — | — |
| 447 | — | — | — | — | — |
| 448 | — | — | — | — | — |
| 449 | — | — | — | — | — |
| 450 | — | — | — | — | — |
| 451 | — | — | — | — | — |
| 452 | — | — | — | — | — |
| 453 | — | — | — | — | — |
| 454 | — | — | — | — | — |
| 455 | — | — | — | — | — |
| 456 | — | — | — | — | — |
| 457 | — | — | — | — | — |

TABLE 8
Potency of Exemplary Sequences of the
Disclosure in Non-human Primates

| Duplex | | % mRNA remaining | | | | |
|---|---|---|---|---|---|---|
| ID | | Pre-dose | Week 2 | Week 4 | Week 8 | Week 12 |
| PBS | Mean | 100.00 | 112.95 | 83.34 | 104.58 | 105.71 |
| (n = 3) | SD | 57.61 | 10.77 | 21.20 | 19.15 | 21.43 |
| Duplex | Mean | 100.00 | 18.05 | 14.90 | 32.44 | 39.82 |
| 537 | | | | | | |
| (n = 4) | SD | 34.27 | 7.28 | 7.48 | 16.19 | 16.12 |
| Duplex | Mean | 100.00 | 8.82 | 8.50 | 32.00 | 63.57 |
| 538 | | | | | | |
| (n = 4) | SD | 31.81 | 8.84 | 3.38 | 30.92 | 48.79 |
| Duplex | Mean | 100.00 | 8.17 | 9.17 | 19.70 | 30.11 |
| 539 | | | | | | |
| (n = 4) | SD | 33.06 | 1.84 | 4.93 | 5.76 | 16.26 |
| Duplex | Mean | 100.00 | 10.16 | 6.26 | 22.60 | 24.16 |
| 540 | | | | | | |
| (n = 4) | SD | 27.82 | 2.17 | 0.76 | 6.07 | 5.46 |
| Duplex | Mean | 100.00 | 8.54 | 8.59 | 26.82 | 24.80 |
| 541 | | | | | | |
| (n = 4) | SD | 33.96 | 4.23 | 3.68 | 22.55 | 2.47 |

TABLE 9
Potency of Exemplary Sequences of the
Disclosure in Non-human Primates

| Duplex | | % mRNA remaining | | | | |
|---|---|---|---|---|---|---|
| ID | | Pre-dose | Day 14 | Day 28 | Day 56 | Day 84 |
| PBS | Mean | 100.00 | 75.07 | 121.11 | 121.38 | 109.04 |
| (n = 2) | SD | 38.47 | 6.41 | 28.98 | 12.61 | 10.25 |
| Duplex | Mean | 100.00 | 32.38 | 27.11 | 37.50 | 42.44 |
| 540 | | | | | | |
| (n = 3) | SD | 42.14 | 16.02 | 10.98 | 22.00 | 25.25 |
| Duplex | Mean | 100.00 | 21.06 | 17.40 | 32.73 | 35.82 |
| 542 | | | | | | |
| (n = 3) | SD | 42.44 | 9.73 | 1.53 | 1.83 | 7.53 |

1. An isolated double stranded oligonucleotide comprising an antisense strand comprising a sequence selected from SEQ ID NOs: 2-269 and its corresponding sense strand comprising a sequence selected from SEQ ID NOs: 538-805, as set forth in Table 5.

2. The isolated double stranded oligonucleotide of claim 1, wherein the antisense strand comprises SEQ ID NO: 58 and its corresponding sense strand comprises SEQ ID NO: 594.

3. The isolated double stranded oligonucleotide of claim 1, wherein the antisense strand comprises SEQ ID NO: 95 and its corresponding sense strand comprises SEQ ID NO: 631.

4. The isolated double stranded oligonucleotide of claim 1, wherein the antisense strand comprises SEQ ID NO: 36 and its corresponding sense strand comprises SEQ ID NO: 572.

5. The isolated double stranded oligonucleotide of claim 1, wherein the antisense strand comprises SEQ ID NO: 21 and its corresponding sense strand comprises SEQ ID NO: 557.

6. The isolated double stranded oligonucleotide of claim 1, wherein the antisense strand comprises SEQ ID NO: 61 and its corresponding sense strand comprises SEQ ID NO: 597.

7. The isolated double stranded oligonucleotide of claim 1, wherein the sense strand or the antisense strand or both comprise one or more modified nucleotide(s).

8. The isolated double stranded oligonucleotide of claim 7, wherein the antisense strand comprises a modified sequence selected from SEQ ID NOs: 270-537 and SEQ ID NOs: 1074-1078, and its corresponding sense strand comprises a modified sequence selected from SEQ ID NOs: 806-1073 and SEQ ID NOs: 1079-1084, as set forth in Table 6.

9. The isolated double stranded oligonucleotide of claim 8, wherein the antisense strand comprises SEQ ID NO: 1074 and its corresponding sense strand comprises SEQ ID NO: 1079.

10. The isolated double stranded oligonucleotide of claim 8, wherein the antisense strand comprises SEQ ID NO: 1075 and its corresponding sense strand comprises SEQ ID NO: 1080.

11. The isolated double stranded oligonucleotide of claim 8, wherein the antisense strand comprises SEQ ID NO: 1076 and its corresponding sense strand comprises SEQ ID NO: 1081.

12. The isolated double stranded oligonucleotide of claim 8, wherein the antisense strand comprises SEQ ID NO: 1077 and its corresponding sense strand comprises SEQ ID NO: 1082.

13. The isolated double stranded oligonucleotide of claim 8, wherein the antisense strand comprises SEQ ID NO: 1078 and its corresponding sense strand comprises SEQ ID NO: 1083.

14. The isolated double stranded oligonucleotide of claim 8, wherein the antisense strand comprises SEQ ID NO: 1077 and its corresponding sense strand comprises SEQ ID NO: 1084.

15. The isolated double stranded oligonucleotide of claim 1, wherein the antisense strand and its corresponding sense strand are selected from Table 7 or Table 8.

16. The isolated double stranded oligonucleotide of claim 1, wherein the antisense strand comprises a phosphate mimic.

17. The isolated double stranded oligonucleotide of claim 16, wherein the phosphate mimic is 5′-C-EP.

18. The isolated double stranded oligonucleotide of claim 17, wherein the phosphate mimic is 5′-C-EPmUs.

19. The isolated double stranded oligonucleotide of claim 1, wherein the sense strand comprises nucleotides modified with 2′-F modification, and nucleotides modified with 2′-O-methyl modification, according to the formula:
5'(M)o(F)o(M)5(F)1(M)1(F)4(M)9 3'
wherein “M” is a 2′-O-methyl modified nucleotide, and “F” is a 2′-F modified nucleotide.

20. The isolated double stranded oligonucleotide of claim 1, wherein the antisense strand comprises nucleotides modified with 2′-F modification, and nucleotides modified with 2′-O-methyl modification, according to the formula:
3'(M)o(F)o(M)6(F)1(M)1(F)1(M)3(F)1(M)2(F)1(M)1(F)1(M)1(F)2(M)1 5'
wherein “M” is a 2′-O-methyl modified nucleotide, and “F” is a 2′-F modified nucleotide.

21. The isolated double stranded oligonucleotide of claim 1, wherein the sense strand comprises phosphorothioate internucleotide linkages located between nucleotides at position 1 to 3 and position 19 to 20 from the first nucleotide at the 5′ terminus of the sense strand.

22. The isolated double stranded oligonucleotide of claim 1, wherein the sense strand further comprises phosphorothioate internucleotide linkages located between nucleotides at position 18 to 19 and position 20 to a targeting ligand from the first nucleotide at the 5′ terminus of the sense strand.

23. The isolated double stranded oligonucleotide of claim 1, wherein the antisense strand comprises phosphorothioate internucleotide linkages located between nucleotides at position 1 to 3 and position 20 to 22 from the first nucleotide at the 5′ terminus of the antisense strand.

24. The isolated double stranded oligonucleotide of claim 1, wherein in the sense strand or the antisense strand or both comprise a terminal or internal nucleotide linked to a targeting ligand.

25. The isolated double stranded oligonucleotide of claim 24, wherein the targeting ligand is an adipose-targeting ligand.

26. The isolated double stranded oligonucleotide of claim 25, wherein the adipose-targeting ligand is selected from a small molecule, a peptide, an antibody, or a carbohydrate.

27. The isolated double stranded oligonucleotide of claim 24, wherein the targeting ligand is linked to a terminal nucleotide on the sense strand via a linker.

28. The isolated double stranded oligonucleotide of claim 24, wherein the targeting ligand comprises at least one GalNAc G1b moiety, optionally three G1b moieties.

29. The isolated double stranded oligonucleotide of claim 28, wherein the targeting ligand comprises G1b-BCN-C22PC2.

30. The isolated double stranded oligonucleotide of claim 25, wherein the adipose-targeting ligand comprises a structure of

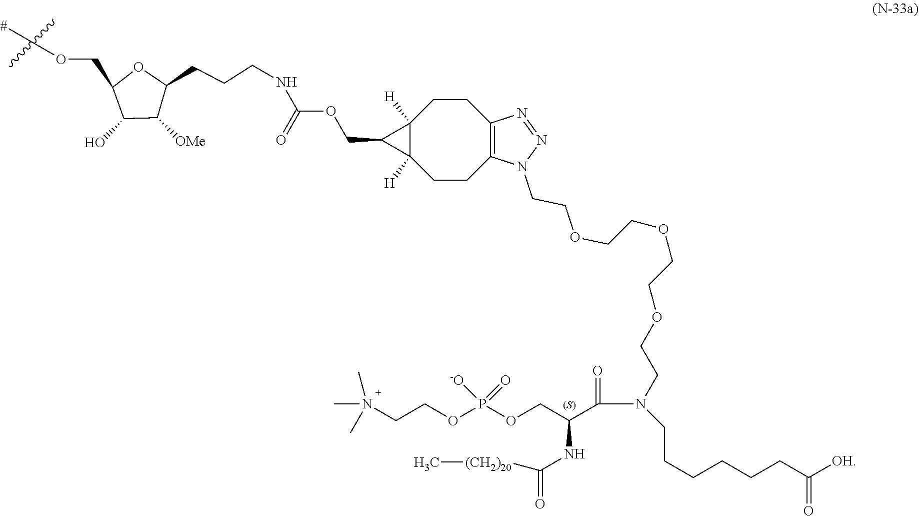

31. (canceled)

32. A pharmaceutical composition comprising at least one isolated double stranded oligonucleotide of claim 1, and a pharmaceutically acceptable carrier, diluent or excipient.

33. A method of inhibiting or downregulating the expression or level of ACVR1C in a subject, wherein the method comprises administering to the subject in need thereof, an effective amount of at least one isolated double stranded oligonucleotide of claim 1.

34. A method of treating or preventing a disease or disorder associated with aberrant or increased expression or activity of ACVR1C or a disease or disorder where ACVR1C plays a role in the disease or disorder, wherein the method comprises administering to a subject in need thereof, an effective amount of at least one isolated double stranded oligonucleotide of claim 1.

35. The method of claim 34, wherein the double stranded oligonucleotide is administered to the subject via an intravenous, a subcutaneous, an intraperitoneal, or an intramuscular route of administration.

36. (canceled)

37. (canceled)
